(19) United States
(12) Patent Application Publication
(10) Pub. No.: US 2026/0083855 A1
(43) Pub. Date: Mar. 26, 2026

(54) IMMUNOCONJUGATES

(71) Applicant: Hoffmann-La Roche Inc., Little Falls, NJ (US)

(72) Inventors: Laura CODARRI DEAK, Au (CH); Anne FREIMOSER-GRUNDSCHOBER, Zurich (CH); Ralf Joerg HOSSE, Mettmenstetten (CH); Christian KLEIN, Bonstetten (CH); Ekkehard MOESSNER, Kreuzlingen (CH); Valeria GONZALEZ NICOLINI, Erlenbach ZH (CH); Pablo UMAÑA FERNÁNDEZ, Lachen (CH); Inja WALDHAUER, Zurich (CH)

(73) Assignee: Hoffmann-La Roche Inc., Little Falls, NJ (US)

(21) Appl. No.: 19/267,102

(22) Filed: Jul. 11, 2025

Related U.S. Application Data
(63) Continuation of application No. PCT/EP2024/051089, filed on Jan. 18, 2024.

(30) Foreign Application Priority Data
Jan. 20, 2023 (EP) 23152578.3

(51) Int. Cl. A61K 47/68; A61K 38/00; A61P 35/00; C07K 14/55; C07K 16/28

(52) U.S. Cl. CPC A61K 47/6813; A61K 47/6849; A61K 47/6889; A61P 35/00; C07K 14/55; C07K 16/2818; A61K 38/00; C07K 2319/00

(57) ABSTRACT

The present invention generally relates to immunoconjugates, particularly immunoconjugates comprising a mutant interleukin-2 polypeptide and an antibody that binds to PD-1. In addition, the invention relates to polynucleotide molecules encoding the immunoconjugates, and vectors and host cells comprising such polynucleotide molecules. The invention further relates to methods for producing the mutant immunoconjugates, pharmaceutical compositions comprising the same, and uses thereof.

### CROSS-REFERENCE TO RELATED APPLICATIONS

This application is a continuation of the International Application No. PCT/EP2024/051089, filed Jan. 18, 2024, which claims the benefit of priority to European Application No. 23152578.3, filed Jan. 20, 2023, each of which is incorporated by reference in its entirety.

### SEQUENCE LISTING

This application contains a Sequence Listing, which has been submitted electronically in XML format and is hereby incorporated by reference in its entirety. Said XML copy, created on Jul. 10, 2025, is named P39036-US-1-Sequence-Listing.xml and is 125,828 bytes in size.

### FIELD OF THE INVENTION

The present invention generally relates to immunoconjugates, particularly immunoconjugates comprising a mutant interleukin-2 polypeptide and an antibody that binds to PD-1. In addition, the invention relates to polynucleotide molecules encoding the immunoconjugates, and vectors and host cells comprising such polynucleotide molecules. The invention further relates to methods for producing the mutant immunoconjugates, pharmaceutical compositions comprising the same, and uses thereof.

### BACKGROUND

Interleukin-2 (IL-2), also known as T cell growth factor (TCGF), is a 15.5 kDa globular glycoprotein playing a central role in lymphocyte generation, survival and homeostasis. It has a length of 133 amino acids and consists of four antiparallel, amphiphatic α-helices that form a quaternary structure indispensable of its function (Smith, Science 240, 1169-76 (1988); Bazan, Science 257, 410-413 (1992)). Sequences of IL-2 from different species are found under NCBI RefSeq Nos. NP000577 (human), NP032392 (mouse), NP446288 (rat) or NP517425 (chimpanzee). IL-2 mediates its action by binding to IL-2 receptors (IL-2R), which consist of up to three individual subunits, the different association of which can produce receptor forms that differ in their affinity to IL-2. Association of the α (CD25), β (CD122), and γ (γ_{c}, CD132) subunits results in a trimeric, high-affinity receptor for IL-2. Dimeric IL-2 receptor consisting of the β and γ subunits is termed intermediate-affinity IL-2R. The α subunit forms the monomeric low affinity IL-2 receptor. Although the dimeric intermediate-affinity IL-2 receptor binds IL-2 with approximately 100-fold lower affinity than the trimeric high-affinity receptor, both the dimeric and the trimeric IL-2 receptor variants are able to transmit signal upon IL-2 binding (Minami et al., Annu Rev Immunol 11, 245-268 (1993)). Hence, the α-subunit, CD25, is not essential for IL-2 signalling. It confers high-affinity binding to its receptor, whereas the β subunit, CD122, and the γ-subunit are crucial for signal transduction (Krieg et al., Proc Natl Acad Sci 107, 11906-11 (2010)). Trimeric IL-2 receptors including CD25 are expressed by (resting) CD4⁺ forkhead box P3 (FoxP3)⁺ regulatory T (T_{reg}) cells. They are also transiently induced on conventional activated T cells, whereas in the resting state these cells express only dimeric IL-2 receptors. T_{reg} cells consistently express the highest level of CD25 in vivo (Fontenot et al., Nature Immunol 6, 1142-51(2005)).

IL-2 is synthesized mainly by activated T-cells, in particular CD4⁺ helper T cells. It stimulates the proliferation and differentiation of T cells, induces the generation of cytotoxic T lymphocytes (CTLs) and the differentiation of peripheral blood lymphocytes to cytotoxic cells and lymphokine-activated killer (LAK) cells, promotes cytokine and cytolytic molecule expression by T cells, facilitates the proliferation and differentiation of B-cells and the synthesis of immunoglobulin by B-cells, and stimulates the generation, proliferation and activation of natural killer (NK) cells (reviewed e.g. in Waldmann, Nat Rev Immunol 6, 595-601 (2009); Olejniczak and Kasprzak, Med Sci Monit 14, RA179-89 (2008); Malek, Annu Rev Immunol 26, 453-79 (2008)).

Its ability to expand lymphocyte populations in vivo and to increase the effector functions of these cells confers antitumor effects to IL-2, making IL-2 immunotherapy an attractive treatment option for certain metastatic cancers. Consequently, high-dose IL-2 treatment has been approved for use in patients with metastatic renal-cell carcinoma and malignant melanoma.

However, IL-2 has a dual function in the immune response in that it not only mediates expansion and activity of effector cells, but also is crucially involved in maintaining peripheral immune tolerance.

A major mechanism underlying peripheral self-tolerance is IL-2 induced activation-induced cell death (AICD) in T cells. AICD is a process by which fully activated T cells undergo programmed cell death through engagement of cell surface-expressed death receptors such as CD95 (also known as Fas) or the TNF receptor. When antigen-activated T cells expressing a high-affinity IL-2 receptor (after previous exposure to IL-2) during proliferation are re-stimulated with antigen via the T cell receptor (TCR)/CD3 complex, the expression of Fas ligand (FasL) and/or tumor necrosis factor (TNF) is induced, making the cells susceptible for Fas-mediated apoptosis. This process is IL-2 dependent (Lenardo, Nature 353, 858-61 (1991)) and mediated via STAT5. By the process of AICD in T lymphocytes tolerance can not only be established to self-antigens, but also to persistent antigens that are clearly not part of the host's makeup, such as tumor antigens.

Moreover, IL-2 is also involved in the maintenance of peripheral CD4⁺CD25⁺ regulatory T (T_{reg}) cells (Fontenot et al., Nature Immunol 6, 1142-51 (2005); D′Cruz and Klein, Nature Immunol 6, 1152-59 (2005); Maloy and Powrie, Nature Immunol 6, 1171-72 (2005), which are also known as suppressor T cells. They suppress effector T cells from destroying their (self-)target, either through cell-cell contact by inhibiting T cell help and activation, or through release of immunosuppressive cytokines such as IL-10 or TGF-β. Depletion of T_{reg} cells was shown to enhance IL-2 induced anti-tumor immunity (Imai et al., Cancer Sci 98, 416-23 (2007)).

Therefore, IL-2 is not optimal for inhibiting tumor growth, because in the presence of IL-2 either the CTLs generated might recognize the tumor as self and undergo AICD or the immune response might be inhibited by IL-2 dependent T_{reg} cells.

A further concern in relation to IL-2 immunotherapy are the side effects produced by recombinant human IL-2 treatment. Patients receiving high-dose IL-2 treatment frequently experience severe cardiovascular, pulmonary, renal, hepatic, gastrointestinal, neurological, cutaneous, haematological and systemic adverse events, which require intensive monitoring and in-patient management. The majority of these side effects can be explained by the development of so-called vascular (or capillary) leak syndrome (VLS), a pathological increase in vascular permeability leading to fluid extravasation in multiple organs (causing e.g. pulmonary and cutaneous edema and liver cell damage) and intravascular fluid depletion (causing a drop in blood pressure and compensatory increase in heart rate). There is no treatment of VLS other than withdrawal of IL-2. Low-dose IL-2 regimens have been tested in patients to avoid VLS, however, at the expense of suboptimal therapeutic results. VLS was believed to be caused by the release of proinflammatory cytokines, such as tumor necrosis factor (TNF)-α from IL-2-activated NK cells, however it has recently been shown that IL-2-induced pulmonary edema resulted from direct binding of IL-2 to lung endothelial cells, which expressed low to intermediate levels of functional αβγ IL-2 receptors (Krieg et al., Proc Nat Acad Sci USA 107, 11906-11 (2010)).

Several approaches have been taken to overcome these problems associated with IL-2 immunotherapy. For example, it has been found that the combination of IL-2 with certain anti-IL-2 monoclonal antibodies enhances treatment effects of IL-2 in vivo (Kamimura et al., J Immunol 177, 306-14 (2006); Boyman et al., Science 311, 1924-27 (2006)). In an alternative approach, IL-2 has been mutated in various ways to reduce its toxicity and/or increase its efficacy. Hu et al. (Blood 101, 4853-4861 (2003), US Pat. Publ. No. 2003/0124678) have substituted the arginine residue in position 38 of IL-2 by tryptophan to eliminate IL-2's vasopermeability activity. Shanafelt et al. (Nature Biotechnol 18, 1197-1202 (2000)) have mutated asparagine 88 to arginine to enhance selectivity for T cells over NK cells. Heaton et al. (Cancer Res 53, 2597-602 (1993); U.S. Pat. No. 5,229,109) have introduced two mutations, Arg38Ala and Phe42Lys, to reduce the secretion of proinflammatory cytokines from NK cells. Gillies et al. (US Pat. Publ. No. 2007/0036752) have substituted three residues of IL-2 (Asp20Thr, Asn88Arg, and Gln126Asp) that contribute to affinity for the intermediate-affinity IL-2 receptor to reduce VLS. Gillies et al. (WO 2008/0034473) have also mutated the interface of IL-2 with CD25 by amino acid substitution Arg38Trp and Phe42Lys to reduce interaction with CD25 and activation of T_{reg} cells for enhancing efficacy. To the same aim, Wittrup et al. (WO 2009/061853) have produced IL-2 mutants that have enhanced affinity to CD25, but do not activate the receptor, thus act as antagonists. The mutations introduced were aimed at disrupting the interaction with the β- and/or γ-subunit of the receptor.

A particular mutant IL-2 polypeptide, designed to overcome the above-mentioned problems associated with IL-2 immunotherapy (toxicity caused by the induction of VLS, tumor tolerance caused by the induction of AICD, and immunosuppression caused by activation of T_{reg} cells), is described in WO 2012/107417. Substitution of the phenylalanine residue at position 42 by alanine, the tyrosine residue at position 45 by alanine and the leucine residue at position 72 of IL-2 by glycine essentially abolishes binding of this mutant IL-2 polypeptide to the α-subunit of the IL-2 receptor (CD25). Liu et al. describe engineered interleukin antagonists (Liu et al., J Immunother. 2009; 32(9): 887-894).

Further to the above-mentioned approaches, IL-2 immunotherapy may be improved by selectively targeting IL-2 to tumors, e.g. in the form of immunoconjugates comprising an antibody that binds to an antigen expressed on tumor cells. Several such immunoconjugates have been described (see e.g. Ko et al., J Immunother (2004) 27, 232-239; Klein et al., Oncoimmunology (2017) 6(3), e1277306).

Tumors may be able, however, to escape such targeting by shedding, mutating or downregulating the target antigen of the antibody. Moreover, tumor-targeted IL-2 may not come into optimal contact with effector cells such as cytotoxic T lymphocytes (CTLs), in tumor microenvironments that actively exclude lymphocytes.

Thus there remains a need to further improve IL-2 immunotherapy. An approach, which may circumvent the problems of tumor-targeting, is to target IL-2 directly to effector cells, in particular CTLs.

Ghasemi et al. have described a fusion protein of IL-2 and an NKG2D binding protein (Ghashemi et al., Nat Comm (2016) 7, 12878), for targeting IL-2 to NKG2D-bearing cells such as natural killer (NK) cells.

Programmed cell death protein 1 (PD-1 or CD279) is an inhibitory member of the CD28 family of receptors, that also includes CD28, CTLA-4, ICOS and BTLA. PD-1 is a cell surface receptor and is expressed on activated B cells, T cells, and myeloid cells (Okazaki et al (2002) Curr. Opin. Immunol. 14: 391779-82; Bennett et al. (2003) J Immunol 170:711-8). The structure of PD-1 is a monomeric type 1 transmembrane protein, consisting of one immunoglobulin variable-like extracellular domain and a cytoplasmic domain containing an immunoreceptor tyrosine-based inhibitory motif (ITIM) and an immunoreceptor tyrosine-based switch motif (ITSM). Two ligands for PD-1 have been identified, PD-L1 and PD-L2, that have been shown to downregulate T cell activation upon binding to PD-1 (Freeman et al (2000) J Exp Med 192: 1027-34; Latchman et al (2001) Nat Immunol 2:261-8; Carter et al (2002) Eur J Immunol 32:634-43). Both PD-L1 and PD-L2 are B7 homologs that bind to PD-1, but do not bind to other CD28 family members. One ligand for PD-1, PD-L1 is abundant in a variety of human cancers (Dong et al (2002) Nat. Med 8:787-9). The interaction between PD-1 and PD-L1 results in a decrease in tumor infiltrating lymphocytes, a decrease in T-cell receptor mediated proliferation, and immune evasion by the cancerous cells (Dong et al. (2003) J. MoI. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1, and the effect is additive when the interaction of PD-1 with PD-L2 is blocked as well (Iwai et al. (2002) Proc. Nat 7. Acad. ScL USA 99: 12293-7; Brown et al. (2003) J. Immunol. 170:1257-66).

Antibodies that bind to PD-1 are described e.g. in WO 2017/055443 A1. Immunoconjugates that bind to PD-1 are described e.g. in WO 2018/184964 A1.

### SUMMARY OF THE INVENTION

The present invention provides a novel approach of targeting a mutant form of IL-2 with advantageous properties for immunotherapy directly to immune effector cells, such as cytotoxic T lymphocytes, rather than tumor cells. Targeting to immune effector cells is achieved by conjugation of the mutant IL-2 molecule to an antibody that binds to PD-1.

The IL-2 mutant used in the present invention has been designed to overcome the problems associated with IL-2 immunotherapy, in particular toxicity caused by the induction of VLS, tumor tolerance caused by the induction of AICD, and immunosuppression caused by activation of T_{reg} cells. In addition to circumventing escape of tumors from tumor-targeting as mentioned above, targeting of the IL-2 mutant to immune effector cells may further increase the preferential activation of CTLs over immunosuppressive T_{reg} cells. By using an antibody that binds to PD-1, the suppression of T-cell activity induced by the interaction of PD-1 with its ligand PD-L1 may additionally be reversed, thus further enhancing the immune response.

An IL-2 fusion protein comprising the anti-PD-L1 antibody atezolizumab has been described by Chen et al. (Chen et al., Biochem Biophys Res Comm (2016) 480, 160-165).

Of note, the immunoconjugate of the invention, comprising an antibody that binds to PD-1, shows significantly superior anti-tumor efficacy in vivo as compared to a similar immunoconjugate targeting PD-L1 (see Example 4 hereinbelow).

In a first aspect, the invention provides an immunoconjugate comprising a mutant IL-2 polypeptide and an antibody that binds to PD-1, wherein the mutant IL-2 polypeptide is a human IL-2 molecule comprising the amino acid substitutions F42A, Y45A, L72G and Q126T (numbering relative to the human IL-2 sequence SEQ ID NO: 90).

In a further aspect, the invention provides an immunoconjugate comprising a mutant IL-2 polypeptide and an antibody that binds to PD-1, wherein the mutant IL-2 polypeptide is a human IL-2 molecule comprising the amino acid substitutions F42A, Y45A, L72G and Q126T (numbering relative to the human IL-2 sequence SEQ ID NO: 90); and wherein the antibody comprises (a) a heavy chain variable region (VH) comprising a CDRR-H1 comprising the amino acid sequence of SEQ ID NO:74, a CDR-H2 comprising the amino acid sequence of SEQ ID NO:75, a CDR-H3 comprising the amino acid sequence of SEQ ID NO:76, and (b) a light chain variable region (VL) comprising a CDR-L1 comprising the amino acid sequence of SEQ ID NO:77, a CDR-L2 comprising the amino acid sequence of SEQ ID NO:78, and a CDR-L3 comprising the amino acid sequence of SEQ ID NO:79.

In another aspect, the invention provides an immunoconjugate comprising a mutant IL-2 polypeptide and an antibody that binds to PD-1, wherein the mutant IL-2 polypeptide is a human IL-2 molecule comprising the amino acid substitutions F42A, Y45A, L72G and Q126T (numbering relative to the human IL-2 sequence SEQ ID NO: 90); and wherein the antibody comprises (a) a heavy chain variable region (VH) comprising an amino acid sequence that is at least about 95%, 96%, 97%, 98%, 99% or 100% identical to the amino acid sequence of SEQ ID NO:80, and (b) a light chain variable region (VL) comprising an amino acid sequence that is at least about 95%, 96%, 97%, 98%, 99% or 100% identical to an amino acid sequence of SEQ ID NO:81.

In some embodiments of the immunoconjugate according to the invention, the mutant IL-2 polypeptide further comprises the amino acid substitution T3A and/or the amino acid substitution C125A. In some embodiments, the mutant IL-2 polypeptide comprises the sequence of SEQ ID NO: 92. In some embodiments, the immunoconjugate comprises not more than one mutant IL-2 polypeptide. In some embodiments, the antibody comprises an Fc domain composed of a first and a second subunit. In some such embodiments, the Fc domain is an IgG class, particularly an IgG₁ subclass, Fc domain, and/or the Fc domain is a human Fc domain. In some embodiments, the antibody is an IgG class, particularly an IgG₁ subclass immunoglobulin.

In some embodiments wherein the immunoconjugate comprises an Fc domain, the Fc domain comprises a modification promoting the association of the first and the second subunit of the Fc domain. In some embodiments, in the CH3 domain of the first subunit of the Fc domain an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable. In some embodiments, in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numberings according to Kabat EU index). In some such embodiments, in the first subunit of the Fc domain additionally the serine residue at position 354 is replaced with a cysteine residue (S354C) or the glutamic acid residue at position 356 is replaced with a cysteine residue (E356C), and in the second subunit of the Fc domain additionally the tyrosine residue at position 349 is replaced by a cysteine residue (Y349C) (numberings according to Kabat EU index). In some embodiments, the mutant IL-2 polypeptide is fused at its amino-terminal amino acid to the carboxy-terminal amino acid of one of the subunits of the Fc domain, particularly the first subunit of the Fc domain, optionally through a linker peptide. In some such embodiments, the linker peptide has the amino acid sequence of SEQ ID NO:93.

In some embodiments wherein the immunoconjugate comprises an Fc domain, the Fc domain comprises one or more amino acid substitution that reduces binding to an Fc receptor, particularly an Fey receptor, and/or effector function, particularly antibody-dependent cell-mediated cytotoxicity (ADCC). In some such embodiments, said one or more amino acid substitution is at one or more position selected from the group of L234, L235, and P329 (Kabat EU index numbering). In some embodiments, each subunit of the Fc domain comprises the amino acid substitutions L234A, L235A and P329G (Kabat EU index numbering).

In some embodiments, the immunoconjugate according to the invention comprises a polypeptide comprising an amino acid sequence that is at least about 80%, 85%, 90%, 95%, 96%, 97%, 98%, 99% or 100% identical to the sequence of SEQ ID NO:21, a polypeptide comprising an amino acid sequence that is at least about 80%, 85%, 90%, 95%, 96%, 97%, 98%, 99% or 100% identical to the sequence of SEQ ID NO:23 or SEQ ID NO:22, and a polypeptide comprising an amino acid sequence that is at least about 80%, 85%, 90%, 95%, 96%, 97%, 98%, 99% or 100% identical to the sequence of SEQ ID NO:35. In some embodiments, the immunoconjugate essentially consists of a mutant IL-2 polypeptide and an IgG₁ immunoglobulin molecule, joined by a linker sequence.

The invention further provides one or more isolated polynucleotide encoding an immunoconjugate of the invention, one or more vector (particularly expression vector) comprising said polynucleotides, and host cells comprising said polynucleotide(s) or said vector(s).

Also provided is a method of producing an immunoconjugate comprising a mutant IL-2 polypeptide and an antibody that binds to PD-1, comprising (a) culturing the host cell of the invention under conditions suitable for the expression of the immunoconjugate, and optionally (b) recovering the immunoconjugate. Also provided by the invention is an immunoconjugate comprising a mutant IL-2 polypeptide and an antibody that binds to PD-1, produced by said method.

The invention further provides a pharmaceutical composition comprising an immunoconjugate of the invention and a pharmaceutically acceptable carrier, and methods of using an immunoconjugate of the invention.

In particular, the invention encompasses an immunoconjugate according to the invention for use as a medicament, and for use in the treatment of a disease. In a particular embodiment, said disease is cancer.

Also encompassed by the invention is the use of an immunoconjugate according to the invention in the manufacture of a medicament for the treatment of a disease. In a particular embodiment, said disease is cancer.

Further provided is a method of treating disease in an individual, comprising administering to said individual a therapeutically effective amount of a composition comprising an immunoconjugate according to the invention in a pharmaceutically acceptable form. In a particular embodiment, said disease is cancer.

Also provided is a method of stimulating the immune system of an individual, comprising administering to said individual an effective amount of a composition comprising an immunoconjugate according to the invention in a pharmaceutically acceptable form.

### DETAILED DESCRIPTION OF THE INVENTION

### Definitions

Terms are used herein as generally used in the art, unless otherwise defined in the following.

The term “interleukin-2” or “IL-2” as used herein, refers to any native IL-2 from any vertebrate source, including mammals such as primates (e.g. humans) and rodents (e.g., mice and rats), unless otherwise indicated. The term encompasses unprocessed IL-2 as well as any form of IL-2 that results from processing in the cell. The term also encompasses naturally occurring variants of IL-2, e.g. splice variants or allelic variants. The amino acid sequence of an exemplary human IL-2 is shown in SEQ ID NO: 90. Unprocessed human IL-2 additionally comprises an N-terminal 20 amino acid signal peptide having the sequence of SEQ ID NO: 94, which is absent in the mature IL-2 molecule.

The term “IL-2 mutant” or “mutant IL-2 polypeptide” as used herein is intended to encompass any mutant forms of various forms of the IL-2 molecule including full-length IL-2, truncated forms of IL-2 and forms where IL-2 is linked to another molecule such as by fusion or chemical conjugation. “Full-length” when used in reference to IL-2 is intended to mean the mature, natural length IL-2 molecule. For example, full-length human IL-2 refers to a molecule that has 133 amino acids (see e.g. SEQ ID NO: 90). The various forms of IL-2 mutants are characterized in having a at least one amino acid mutation affecting the interaction of IL-2 with CD25. This mutation may involve substitution, deletion, truncation or modification of the wild-type amino acid residue normally located at that position. Mutants obtained by amino acid substitution are preferred. Unless otherwise indicated, an IL-2 mutant may be referred to herein as a mutant IL-2 peptide sequence, a mutant IL-2 polypeptide, a mutant IL-2 protein or a mutant IL-2 analog.

Designation of various forms of IL-2 is herein made with respect to the sequence shown in SEQ ID NO: 90. Various designations may be used herein to indicate the same mutation. For example a mutation from phenylalanine at position 42 to alanine can be indicated as 42A, A42, A42, F42A, or Phe42Ala.

By a “human IL-2 molecule” as used herein is meant an IL-2 molecule comprising an amino acid sequence that is at least about 90%, at least about 91%, at least about 92%, at least about 93%, at least about 94%, at least about 95% or at least about 96% identical to the human IL-2 sequence of SEQ ID NO:90. Particularly, the sequence identity is at least about 95%, more particularly at least about 96%. In particular embodiments, the human IL-2 molecule is a full-length IL-2 molecule.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g. reduced binding to CD25. Amino acid sequence deletions and insertions include amino- and/or carboxy-terminal deletions and insertions of amino acids. An example of a terminal deletion is the deletion of the alanine residue in position 1 of full-length human IL-2. Preferred amino acid mutations are amino acid substitutions. For the purpose of altering e.g. the binding characteristics of an IL-2 polypeptide, non-conservative amino acid substitutions, i.e. replacing one amino acid with another amino acid having different structural and/or chemical properties, are particularly preferred. Preferred amino acid substations include replacing a hydrophobic by a hydrophilic amino acid. Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful.

As used herein, a “wild-type” form of IL-2 is a form of IL-2 that is otherwise the same as the mutant IL-2 polypeptide except that the wild-type form has a wild-type amino acid at each amino acid position of the mutant IL-2 polypeptide. For example, if the IL-2 mutant is the full-length IL-2 (i.e. IL-2 not fused or conjugated to any other molecule), the wild-type form of this mutant is full-length native IL-2. If the IL-2 mutant is a fusion between IL-2 and another polypeptide encoded downstream of IL-2 (e.g. an antibody chain) the wild-type form of this IL-2 mutant is IL-2 with a wild-type amino acid sequence, fused to the same downstream polypeptide. Furthermore, if the IL-2 mutant is a truncated form of IL-2 (the mutated or modified sequence within the non-truncated portion of IL-2) then the wild-type form of this IL-2 mutant is a similarly truncated IL-2 that has a wild-type sequence. For the purpose of comparing IL-2 receptor binding affinity or biological activity of various forms of IL-2 mutants to the corresponding wild-type form of IL-2, the term wild-type encompasses forms of IL-2 comprising one or more amino acid mutation that does not affect IL-2 receptor binding compared to the naturally occurring, native IL-2, such as e.g. a substitution of cysteine at a position corresponding to residue 125 of human IL-2 to alanine. In some embodiments wild-type IL-2 for the purpose of the present invention comprises the amino acid substitution C125A. In certain embodiments according to the invention the wild-type IL-2 polypeptide to which the mutant IL-2 polypeptide is compared comprises the amino acid sequence of SEQ ID NO: 90. In other embodiments the wild-type IL-2 polypeptide to which the mutant IL-2 polypeptide is compared comprises the amino acid sequence of SEQ ID NO: 95.

The term “CD25” or “α-subunit of the IL-2 receptor” as used herein, refers to any native CD25 from any vertebrate source, including mammals such as primates (e.g. humans) and rodents (e.g., mice and rats), unless otherwise indicated. The term encompasses “full-length”, unprocessed CD25 as well as any form of CD25 that results from processing in the cell. The term also encompasses naturally occurring variants of CD25, e.g. splice variants or allelic variants. In certain embodiments CD25 is human CD25. The amino acid sequence of human CD25 is found e.g. in UniProt entry no. P01589 (version 185).

The term “high-affinity IL-2 receptor” as used herein refers to the heterotrimeric form of the IL-2 receptor, consisting of the receptor γ-subunit (also known as common cytokine receptor γ-subunit, γ_{c}, or CD132, see UniProt entry no. P14784 (version 192)), the receptor β-subunit (also known as CD122 or p70, see UniProt entry no. P31785 (version 197)) and the receptor α-subunit (also known as CD25 or p55, see UniProt entry no. P01589 (version 185)). The term “intermediate-affinity IL-2 receptor” by contrast refers to the IL-2 receptor including only the γ-subunit and the β-subunit, without the α-subunit (for a review see e.g. Olejniczak and Kasprzak, Med Sci Monit 14, RA179-189 (2008)).

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well established methods known in the art, including those described herein. A particular method for measuring affinity is Surface Plasmon Resonance (SPR).

The affinity of the mutant or wild-type IL-2 polypeptide for various forms of the IL-2 receptor can be determined in accordance with the method set forth in the WO 2012/107417 by surface plasmon resonance (SPR), using standard instrumentation such as a BIAcore instrument (Cytiva) and receptor subunits such as may be obtained by recombinant expression (see e.g. Shanafelt et al., Nature Biotechnol 18, 1197-1202 (2000)). Alternatively, binding affinity of IL-2 mutants for different forms of the IL-2 receptor may be evaluated using cell lines known to express one or the other such form of the receptor. Specific illustrative and exemplary embodiments for measuring binding affinity are described hereinafter.

By “regulatory T cell” or “T_{reg} cell” is meant a specialized type of CD4⁺ T cell that can suppress the responses of other T cells. T_{reg} cells are characterized by expression of the α-subunit of the IL-2 receptor (CD25) and the transcription factor forkhead box P3 (FOXP3) (Sakaguchi, Annu Rev Immunol 22, 531-62 (2004)) and play a critical role in the induction and maintenance of peripheral self-tolerance to antigens, including those expressed by tumors. T_{reg} cells require IL-2 for their function and development and induction of their suppressive characteristics.

As used herein, the term “effector cells” refers to a population of lymphocytes that mediate the cytotoxic effects of IL-2. Effector cells include effector T cells such as CD8+ cytotoxic T cells, NK cells, lymphokine-activated killer (LAK) cells and macrophages/monocytes.

As used herein, the term “PD1”, “human PD1”, “PD-1” or “human PD-1” (also known as Programmed cell death protein 1, or Programmed Death 1) refers to the human protein PD1 (SEQ ID NO: 96, protein without signal sequence)/(SEQ ID NO: 97, protein with signal sequence). See also UniProt entry no. Q15116 (version 156). As used herein, an antibody “binding to PD-1”, “specifically binding to PD-1”, “that binds to PD-1” or “anti-PD-1 antibody” refers to an antibody that is capable of binding PD-1, especially a PD-1 polypeptide expressed on a cell surface, with sufficient affinity such that the antibody is useful as a diagnostic and/or therapeutic agent in targeting PD-1. In one embodiment, the extent of binding of an anti-PD-1 antibody to an unrelated, non-PD-1 protein is less than about 10% of the binding of the antibody to PD-1 as measured, e.g., by radioimmunoassay (RIA) or flow cytometry (FACS) or by a Surface Plasmon Resonance assay using a biosensor system such as a Biacore® system. In certain embodiments, an antibody that binds to PD-1 has a K_{D} value of the binding affinity for binding to human PD-1 of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). In one embodiment, the KD value of the binding affinity is determined in a Surface Plasmon Resonance assay using the Extracellular domain (ECD) of human PD-1 (PD-1-ECD, see SEQ ID NO: 43) as antigen.

By “specific binding” is meant that the binding is selective for the antigen and can be discriminated from unwanted or non-specific interactions. The ability of an antibody to bind to a specific antigen (e.g. PD-1) can be measured either through an enzyme-linked immunosorbent assay (ELISA) or other techniques familiar to one of skill in the art, e.g. surface plasmon resonance (SPR) technique (analyzed e.g. on a BIAcore instrument) (Liljeblad et al., Glyco J 17, 323-329 (2000)), and traditional binding assays (Heeley, Endocr Res 28, 217-229 (2002)). In one embodiment, the extent of binding of an antibody to an unrelated protein is less than about 10% of the binding of the antibody to the antigen as measured, e.g., by SPR. The antibody comprised in the immunoconjugate described herein specifically binds to PD-1.

As used herein, term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein”, “amino acid chain”, or any other term used to refer to a chain of two or more amino acids, are included within the definition of “polypeptide”, and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

By an “isolated” polypeptide or a variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. For purposes herein, however, % amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36, and is publicly available from http://fasta.bioch.virginia.edu/fasta_www2/fasta_down.shtml. Alternatively, a public server accessible at http://fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

The term “polynucleotide” refers to an isolated nucleic acid molecule or construct, e.g. messenger RNA (mRNA), virally-derived RNA, or plasmid DNA (pDNA). A polynucleotide may comprise a conventional phosphodiester bond or a non-conventional bond (e.g. an amide bond, such as found in peptide nucleic acids (PNA). The term “nucleic acid molecule” refers to any one or more nucleic acid segments, e.g. DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

“Isolated polynucleotide (or nucleic acid) encoding [e.g. an immunoconjugate of the invention]” refers to one or more polynucleotide molecules encoding antibody heavy and light chains and/or IL-2 polypeptides (or fragments thereof), including such polynucleotide molecule(s) in a single vector or separate vectors, and such nucleic acid molecule(s) present at one or more locations in a host cell.

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette comprises polynucleotide sequences that encode immunoconjugates of the invention or fragments thereof.

The term “vector” or “expression vector” refers to a DNA molecule that is used to introduce and direct the expression of a specific gene to which it is operably associated in a cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the cell, the ribonucleic acid molecule or protein that is encoded by the gene is produced by the cellular transcription and/or translation machinery. In one embodiment, the expression vector of the invention comprises an expression cassette that comprises polynucleotide sequences that encode immunoconjugates of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the immunoconjugates of the present invention. Host cells include cultured cells, e.g. mammalian cultured cells, such as HEK cells, CHO cells, BHK cells, NSO cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

The term “antibody” exhibits antigen-binding activity and encompasses various antibody structures exhibiting antigen-binding activity, including but not limited to monoclonal antibodies, polyclonal antibodies, multispecific antibodies (e.g., bispecific antibodies), and antibody fragments.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

An “isolated” antibody is one which has been separated from a component of its natural environment, i.e. that is not in its natural milieu. No particular level of purification is required.

For example, an isolated antibody can be removed from its native or natural environment. Recombinantly produced antibodies expressed in host cells are considered isolated for the purpose of the invention, as are native or recombinant antibodies which have been separated, fractionated, or partially or substantially purified by any suitable technique. As such, the immunoconjugates of the present invention are isolated. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For review of methods for assessment of antibody purity, see, e.g., Flatman et al., J. Chromatogr. B 848:79-87 (2007).

The terms “full-length antibody,” “intact antibody,” and “whole antibody” are used herein interchangeably to refer to an antibody having a structure substantially similar to a native antibody structure.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g., scFv, and scFab); single domain antibodies (dAbs); and multispecific antibodies formed from antibody fragments.

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ε (IgE), γ (IgG), or μ (IgM), some of which may be further divided into subtypes, e.g. γ₁ (IgG₁), γ₂ (IgG2), γ₃ (IgG3), γ₄ (IgG4), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “antigen binding domain” refers to the part of an antibody that comprises the area which specifically binds to and is complementary to part or all of an antigen. An antigen binding domain may be provided by, for example, one or more antibody variable domains (also called antibody variable regions). Particularly, an antigen binding domain comprises an antibody light chain variable domain (VL) and an antibody heavy chain variable domain (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W.H. Freeman and Co., page 91 (2007). A single VH or VL domain may be sufficient to confer antigen-binding specificity. As used herein in connection with variable region sequences, “Kabat numbering” refers to the numbering system set forth by Kabat et al., Sequences of Proteins of Immunological Interest, 5th Ed. Public Health Service, National Institutes of Health, Bethesda, MD (1991).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991), referred to as “numbering according to Kabat” or “Kabat numbering” herein. Specifically the Kabat numbering system (see pages 647-660 of Kabat, et al., Sequences of Proteins of Immunological Interest, 5th ed., Public Health Service, National Institutes of Health, Bethesda, MD (1991)) is used for the light chain constant domain CL of kappa and lambda isotype and the Kabat EU index numbering system (see pages 661-723) is used for the heavy chain constant domains (CH1, Hinge, CH2 and CH3), which is herein further clarified by referring to “numbering according to Kabat EU index” in this case.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-HT, CDR-H2, CDR-H3), and three in the VL (CDR-LT, CDR-L2, CDR-L3). CDRs are defined by a variety of methods/systems by those skilled in the art. These systems and/or definitions have been developed and refined over a number of years and include Kabat, Chothia, IMGT, AbM, and Contact. The Kabat definition is based on sequence variability and generally is the most commonly used. The Chothia definition is based on the location of the structural loop regions. The IMGT system is based on sequence variability and location within the structure of the variable domain. The AbM definition is a compromise between Kabat and Chothia. The Contact definition is based on analyses of the available antibody crystal structures. Software programs (e.g., abYsis: http://www.abysis.org/abysis/sequence_input/key_annotation/key_annotation.cgi) are available and known to those of skill in the art for analysis of antibody sequences and determination of CDRs.

Exemplary CDRs herein include (numbering of amino acid residues according to the reference cited, i.e. Chothia numbering for the Chothia and Contact definition, Kabat numbering for the Kabat definition and IMGT numbering for the IMGT definition):
(a) hypervariable loops occurring at amino acid residues 26-32 (L1), 50-52 (L2), 91-96 (L3), 26-32 (H1), 53-55 (H2), and 96-101 (H3) according to Chothia and Lesk, J. Mol. Biol. 196:901-917 (1987) (“Chotia definition”);
(b) CDRs occurring at amino acid residues 24-34 (L1), 50-56 (L2), 89-97 (L3), 31-35b (H1), 50-65 (H2), and 95-102 (H3) according to Kabat et al., Sequences of Proteins of Immunological Interest, 5th Ed. Public Health Service, National Institutes of Health, Bethesda, MD (1991) (“Kabat definion”);
(c) antigen contacts occurring at amino acid residues 30-36 (L1), 46-55 (L2), 89-96 (L3), 30-35 (H1), 47-58 (H2), and 93-101 (H3) according to MacCallum et al. J. Mol. Biol. 262: 732-745 (1996) (“Contact definition”); and
(d) (d) CDRs occurring at amino acid residues residues 27-38 (L1), 56-65 (L2), 105-117 (L3), 27-38 (H1), 56-65 (H2), and 105-117 (H3), according to Lefranc et al. Dev. Comp. Immunol. 27: 55-77 (2003) (“IMGT definition”).

Unless otherwise indicated, the CDRs are determined herein according to Kabat et al., supra. One of skill in the art will understand that the CDR designations can also be determined according to Chothia, supra, MacCallum, supra, Lefranc, supra, or any other scientifically accepted definition/system.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following order in VH (or VL): FR1-H1(L)-FR2-H2(L2)-FR3-H3(L3)-FR4.

A “humanized” antibody refers to an antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain (also referred to herein as a “cleaved variant heavy chain”). This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (K447), of the Fc region may or may not be present. Amino acid sequences of heavy chains including Fc domains (or a subunit of an Fc domain as defined herein) are denoted herein without C-terminal glycine-lysine dipeptide if not indicated otherwise. In one embodiment of the invention, a heavy chain including a subunit of an Fc domain as specified herein, comprised in an immunoconjugate according to the invention, comprises an additional C-terminal glycine-lysine dipeptide (G446 and K447, numbering according to EU index of Kabat). In one embodiment of the invention, a heavy chain including a subunit of an Fc domain as specified herein, comprised in an immunoconjuate according to the invention, comprises an additional C-terminal glycine residue (G446, numbering according to EU index of Kabat). Compositions of the invention, such as the pharmaceutical compositions described herein, comprise a population of immunoconjugates of the invention. The population of immunoconjugates may comprise molecules having a full-length heavy chain and molecules having a cleaved variant heavy chain. The population of immunoconjugates may consist of a mixture of molecules having a full-length heavy chain and molecules having a cleaved variant heavy chain, wherein at least 50%, at least 60%, at least 70%, at least 80% or at least 90% of the immunoconjugates have a cleaved variant heavy chain. In one embodiment of the invention, a composition comprising a population of immunoconjugates of the invention comprises an immunoconjugate comprising a heavy chain including a subunit of an Fc domain as specified herein with an additional C-terminal glycine-lysine dipeptide (G446 and K447, numbering according to EU index of Kabat). In one embodiment of the invention, a composition comprising a population of immunoconjugates of the invention comprises an immunoconjugate comprising a heavy chain including a subunit of an Fc domain as specified herein with an additional C-terminal glycine residue (G446, numbering according to EU index of Kabat). In one embodiment of the invention, such a composition comprises a population of immunoconjugates comprised of molecules comprising a heavy chain including a subunit of an Fc domain as specified herein; molecules comprising a heavy chain including a subunit of a Fc domain as specified herein with an additional C-terminal glycine residue (G446, numbering according to EU index of Kabat); and molecules comprising a heavy chain including a subunit of an Fc domain as specified herein with an additional C-terminal glycine-lysine dipeptide (G446 and K447, numbering according to EU index of Kabat). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g. antigen binding moieties) are not the same. In some embodiments the modification promoting association comprises an amino acid mutation in the Fc domain, specifically an amino acid substitution. In a particular embodiment, the modification promoting association comprises a separate amino acid mutation, specifically an amino acid substitution, in each of the two subunits of the Fc domain.

The term “effector functions” when used in reference to antibodies refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

Antibody-dependent cell-mediated cytotoxicity (ADCC) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g. PCT publication no. WO 2006/082515 or PCT publication no. WO 2012/130831).

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

“Reduced binding”, for example reduced binding to an Fc receptor or CD25, refers to a decrease in affinity for the respective interaction, as measured for example by SPR. For clarity, the term includes also reduction of the affinity to zero (or below the detection limit of the analytic method), i.e. complete abolishment of the interaction. Conversely, “increased binding” refers to an increase in binding affinity for the respective interaction.

As used herein, the term “immunoconjugate” refers to a polypeptide molecule that includes at least one IL-2 molecule and at least one antibody. The IL-2 molecule can be joined to the antibody by a variety of interactions and in a variety of configurations as described herein. In particular embodiments, the IL-2 molecule is fused to the antibody via a peptide linker. Particular immunoconjugates according to the invention essentially consist of one IL-2 molecule and an antibody joined by one or more linker sequences.

By “fused” is meant that the components (e.g. an antibody and an IL-2 molecule) are linked by peptide bonds, either directly or via one or more peptide linkers.

As used herein, the terms “first” and “second” with respect to Fc domain subunits etc., are used for convenience of distinguishing when there is more than one of each type of moiety. Use of these terms is not intended to confer a specific order or orientation of the immunoconjugate unless explicitly so stated.

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the composition would be administered.

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, immunoconjugates of the invention are used to delay development of a disease or to slow the progression of a disease.

### Detailed Description of the Embodiments

Mutant IL-2 polypeptide

The immunoconjugates according to the present invention comprise a mutant IL-2 polypeptide having advantageous properties for immunotherapy. In particular, pharmacological properties of IL-2 that contribute to toxicity but are not essential for efficacy of IL-2 are eliminated in the mutant IL-2 polypeptide. Such mutant IL-2 polypeptides are described in detail in WO 2012/107417, which is incorporated herein by reference in its entirety. As discussed above, different forms of the IL-2 receptor consist of different subunits and exhibit different affinities for IL-2. The intermediate-affinity IL-2 receptor, consisting of the β and γ receptor subunits, is expressed on resting effector cells and is sufficient for IL-2 signaling. The high-affinity IL-2 receptor, additionally comprising the α-subunit of the receptor, is mainly expressed on regulatory T (T_{reg}) cells as well as on activated effector cells where its engagement by IL-2 can promote T_{reg}cell-mediated immunosuppression or activation-induced cell death (AICD), respectively. Thus, without wishing to be bound by theory, reducing or abolishing the affinity of IL-2 to the α-subunit of the IL-2 receptor should reduce IL-2 induced downregulation of effector cell function by regulatory T cells and development of tumor tolerance by the process of AICD. On the other hand, maintaining the affinity to the intermediate-affinity IL-2 receptor should preserve the induction of proliferation and activation of effector cells like NK and T cells by IL-2.

The mutant interleukin-2 (IL-2) polypeptide comprised in the immunoconjugate according to the invention comprises at least one amino acid mutation that abolishes or reduces affinity of the mutant IL-2 polypeptide to the α-subunit of the IL-2 receptor and preserves affinity of the mutant IL-2 polypeptide to the intermediate-affinity IL-2 receptor each compared to a wild-type IL-2 polypeptide.

Mutants of human IL-2 (hIL-2) with decreased affinity to CD25 may for example be generated by amino acid substitution at amino acid position 35, 38, 42, 43, 45 or 72 or combinations thereof (numbering relative to the human IL-2 sequence SEQ ID NO: 90). Exemplary amino acid substitutions include K35E, K35A, R38A, R38E, R38N, R38F, R38S, R38L, R38G, R38Y, R38W, F42L, F42A, F42G, F42S, F42T, F42Q, F42E, F42N, F42D, F42R, F42K, K43E, Y45A, Y45G, Y45S, Y45T, Y45Q, Y45E, Y45N, Y45D, Y45R, Y45K, L72G, L72A, L72S, L72T, L72Q, L72E, L72N, L72D, L72R, and L72K. Particular IL-2 mutants useful in the immunoconjugates of the invention comprise an amino acid mutation at an amino acid position corresponding to residue 42, 45, or 72 of human IL-2, or a combination thereof. In one embodiment said amino acid mutation is an amino acid substitution selected from the group of F42A, F42G, F42S, F42T, F42Q, F42E, F42N, F42D, F42R, F42K, Y45A, Y45G, Y45S, Y45T, Y45Q, Y45E, Y45N, Y45D, Y45R, Y45K, L72G, L72A, L72S, L72T, L72Q, L72E, L72N, L72D, L72R, and L72K, more specifically an amino acid substitution selected from the group of F42A, Y45A and L72G. These mutants exhibit substantially similar binding affinity to the intermediate-affinity IL-2 receptor, and have substantially reduced affinity to the α-subunit of the IL-2 receptor and the high-affinity IL-2 receptor compared to a wild-type form of the IL-2 mutant.

Other characteristics of useful mutants may include the ability to induce proliferation of IL-2 receptor-bearing T and/or NK cells, the ability to induce IL-2 signaling in IL-2 receptor-bearing T and/or NK cells, the ability to generate interferon (IFN)-γ as a secondary cytokine by NK cells, a reduced ability to induce elaboration of secondary cytokines—particularly IL-10 and TNF-α—by peripheral blood mononuclear cells (PBMCs), a reduced ability to activate regulatory T cells, a reduced ability to induce apoptosis in T cells, and a reduced toxicity profile in vivo.

Particular mutant IL-2 polypeptides useful in the invention comprise three amino acid mutations that abolish or reduce affinity of the mutant IL-2 polypeptide to the α-subunit of the IL-2 receptor but preserve affinity of the mutant IL-2 polypeptide to the intermediate affinity IL-2 receptor. In one embodiment said three amino acid mutations are at positions corresponding to residue 42, 45 and 72 of human IL-2. In one embodiment said three amino acid mutations are amino acid substitutions. In one embodiment said three amino acid mutations are amino acid substitutions selected from the group of F42A, F42G, F42S, F42T, F42Q, F42E, F42N, F42D, F42R, F42K, Y45A, Y45G, Y45S, Y45T, Y45Q, Y45E, Y45N, Y45D, Y45R, Y45K, L72G, L72A, L72S, L72T, L72Q, L72E, L72N, L72D, L72R, and L72K. In a specific embodiment said three amino acid mutations are amino acid substitutions F42A, Y45A and L72G (numbering relative to the human IL-2 sequence of SEQ ID NO: 90).

Particular mutant IL-2 polypeptides useful in the invention comprise four amino acid mutations that abolish or reduce affinity of the mutant IL-2 polypeptide to the α-subunit of the IL-2 receptor but preserve affinity of the mutant IL-2 polypeptide to the intermediate affinity IL-2 receptor. In one embodiment said three amino acid mutations are at positions corresponding to residue 42, 45, 72 and 126 of human IL-2. In one embodiment said three amino acid mutations are amino acid substitutions. In one embodiment said three amino acid mutations are amino acid substitutions selected from the group of F42A, F42G, F42S, F42T, F42Q, F42E, F42N, F42D, F42R, F42K, Y45A, Y45G, Y45S, Y45T, Y45Q, Y45E, Y45N, Y45D, Y45R, Y45K, L72G, L72A, L72S, L72T, L72Q, L72E, L72N, L72D, L72R, L72K, Q126T. In a specific embodiment said three amino acid mutations are amino acid substitutions F42A, Y45A, L72G and Q126T (numbering relative to the human IL-2 sequence of SEQ ID NO: 90). In a specific embodiment said three amino acid mutations are amino acid substitutions F42A, Y45A, L72G and N88D (numbering relative to the human IL-2 sequence of SEQ ID NO: 90). In a specific embodiment said three amino acid mutations are amino acid substitutions F42A, Y45A, L72G and N88Q (numbering relative to the human IL-2 sequence of SEQ ID NO: 90).

In certain embodiments said amino acid mutation reduces the affinity of the mutant IL-2 polypeptide to the α-subunit of the IL-2 receptor by at least 5-fold, specifically at least 10-fold, more specifically at least 25-fold. In embodiments where there is more than one amino acid mutation that reduces the affinity of the mutant IL-2 polypeptide to the α-subunit of the IL-2 receptor, the combination of these amino acid mutations may reduce the affinity of the mutant IL-2 polypeptide to the α-subunit of the IL-2 receptor by at least 30-fold, at least 50-fold, or even at least 100-fold. In one embodiment said amino acid mutation or combination of amino acid mutations abolishes the affinity of the mutant IL-2 polypeptide to the α-subunit of the IL-2 receptor so that no binding is detectable by surface plasmon resonance.

Substantially similar binding to the intermediate-affinity receptor, i.e. preservation of the affinity of the mutant IL-2 polypeptide to said receptor, is achieved when the IL-2 mutant exhibits greater than about 70% of the affinity of a wild-type form of the IL-2 mutant to the intermediate-affinity IL-2 receptor. IL-2 mutants of the invention may exhibit greater than about 80% and even greater than about 90% of such affinity.

Reduction of the affinity of IL-2 for the α-subunit of the IL-2 receptor in combination with elimination of the O-glycosylation of IL-2 results in an IL-2 protein with improved properties. For example, elimination of the O-glycosylation site results in a more homogenous product when the mutant IL-2 polypeptide is expressed in mammalian cells such as CHO or HEK cells.

Thus, in certain embodiments the mutant IL-2 polypeptide comprises an additional amino acid mutation which eliminates the O-glycosylation site of IL-2 at a position corresponding to residue 3 of human IL-2. In one embodiment said additional amino acid mutation which eliminates the O-glycosylation site of IL-2 at a position corresponding to residue 3 of human IL-2 is an amino acid substitution. Exemplary amino acid substitutions include T3A, T3G, T3Q, T3E, T3N, T3D, T3R, T3K, and T3P. In a specific embodiment, said additional amino acid mutation is the amino acid substitution T3A.

In certain embodiments the mutant IL-2 polypeptide is essentially a full-length IL-2 molecule. In certain embodiments the mutant IL-2 polypeptide is a human IL-2 molecule. In one embodiment the mutant IL-2 polypeptide comprises the sequence of SEQ ID NO: 90 with at least one amino acid mutation that abolishes or reduces affinity of the mutant IL-2 polypeptide to the α-subunit of the IL-2 receptor but preserve affinity of the mutant IL-2 polypeptide to the intermediate affinity IL-2 receptor, compared to an IL-2 polypeptide comprising SEQ ID NO: 90 without said mutation. In another embodiment, the mutant IL-2 polypeptide comprises the sequence of SEQ ID NO: 95 with at least one amino acid mutation that abolishes or reduces affinity of the mutant IL-2 polypeptide to the α-subunit of the IL-2 receptor but preserve affinity of the mutant IL-2 polypeptide to the intermediate affinity IL-2 receptor, compared to an IL-2 polypeptide comprising SEQ ID NO: 95 without said mutation.

In a specific embodiment, the mutant IL-2 polypeptide can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity.

In one embodiment the mutant IL-2 polypeptide has a reduced ability to induce IL-2 signaling in regulatory T cells, compared to a wild-type IL-2 polypeptide. In one embodiment the mutant IL-2 polypeptide induces less activation-induced cell death (AICD) in T cells, compared to a wild-type IL-2 polypeptide. In one embodiment the mutant IL-2 polypeptide has a reduced toxicity profile in vivo, compared to a wild-type IL-2 polypeptide. In one embodiment the mutant IL-2 polypeptide has a prolonged serum half-life, compared to a wild-type IL-2 polypeptide.

A particular mutant IL-2 polypeptide useful in the invention comprises five amino acid substitutions at positions corresponding to residues 3, 42, 45, 72 and 126 of human IL-2. Specific amino acid substitutions are T3A, F42A, Y45A, L72G and Q126T. Another particular mutant IL-2 polypeptide useful in the invention comprises five amino acid substitutions at positions corresponding to residues 3, 42, 45, 72 and 88 of human IL-2. Specific amino acid substitutions are T3A, F42A, Y45A, L72G and N88D. Another particular mutant IL-2 polypeptide useful in the invention comprises five amino acid substitutions at positions corresponding to residues 3, 42, 45, 72 and 88 of human IL-2. Specific amino acid substitutions are T3A, F42A, Y45A, L72G and N88Q.

IL-2 mutants useful in the invention, in addition to having mutations in the region of IL-2 that forms the interface of IL-2 with CD25 or the glycosylation site, also may have one or more mutations in the amino acid sequence outside these regions. Such additional mutations in human IL-2 may provide additional advantages such as increased expression or stability. For example, the cysteine at position 125 may be replaced with a neutral amino acid such as serine, alanine, threonine or valine, yielding C125S IL-2, C125A IL-2, C125T IL-2 or C125V IL-2 respectively, as described in U.S. Pat. No. 4,518,584. As described therein, one may also delete the N-terminal alanine residue of IL-2 yielding such mutants as des-A1 C125S or des-A1 C125A. Alternatively or conjunctively, the IL-2 mutant may include a mutation whereby methionine normally occurring at position 104 of wild-type human IL-2 is replaced by a neutral amino acid such as alanine (see U.S. Pat. No. 5,206,344). The resulting mutants, e. g., des-A1 M104A IL-2, des-A1 M104A C125S IL-2, M104A IL-2, M104A C125A IL-2, des-A1 M104A C125A IL-2, or M104A C125S IL-2 (these and other mutants may be found in U.S. Pat. No. 5,116,943 and in Weiger et al., Eur J Biochem 180, 295-300 (1989)) may be used in conjunction with the particular IL-2 mutations of the invention.

Thus, in certain embodiments the mutant IL-2 polypeptide comprises an additional amino acid mutation at a position corresponding to residue 125 of human IL-2. In one embodiment said additional amino acid mutation is the amino acid substitution C125A.

The skilled person will be able to determine which additional mutations may provide additional advantages for the purpose of the invention. For example, he will appreciate that amino acid mutations in the IL-2 sequence that reduce or abolish the affinity of IL-2 to the intermediate-affinity IL-2 receptor, such as D20T, N88R or Q126D (see e.g. US 2007/0036752), may not be suitable to include in the mutant IL-2 polypeptide according to the invention.

In one embodiment, the mutant IL-2 polypeptide comprises no more than 12, no more than 11, no more than 10, no more than 9, no more than 8, no more than 7, no more than 6, or no more than 5 amino acid mutations as compared to the corresponding wild-type IL-2 sequence, e.g. the human IL-2 sequence of SEQ ID NO: 90. In a particular embodiment, the mutant IL-2 polypeptide comprises no more than 5 amino acid mutations as compared to the corresponding wild-type IL-2 sequence, e.g. the human IL-2 sequence of SEQ ID NO: 90.

In one embodiment the mutant IL-2 polypeptide comprises the sequence of SEQ ID NO: 92. In one embodiment the mutant IL-2 polypeptide consists of the sequence of SEQ ID NO: 92.

In one embodiment the mutant IL-2 polypeptide comprises the sequence of SEQ ID NO: 98. In one embodiment the mutant IL-2 polypeptide consists of the sequence of SEQ ID NO: 98.

In one embodiment the mutant IL-2 polypeptide comprises the sequence of SEQ ID NO: 99. In one embodiment the mutant IL-2 polypeptide consists of the sequence of SEQ ID NO: 99.

### Immunoconjugates

Immunoconjugates as described herein comprise an IL-molecule and an antibody. Such immunoconjugates significantly increase the efficacy of IL-2 therapy by directly targeting IL-2 e.g. into a tumor microenvironment. According to the invention, an antibody comprised in the immunoconjugate can be a whole antibody or immunoglobulin, or a portion or variant thereof that has a biological function such as antigen specific binding affinity.

The general benefits of immunoconjugate therapy are readily apparent. For example, an antibody comprised in an immunoconjugate recognizes a tumor-specific epitope and results in targeting of the immunoconjugate molecule to the tumor site. Therefore, high concentrations of IL-2 can be delivered into the tumor microenvironment, thereby resulting in activation and proliferation of a variety of immune effector cells mentioned herein using a much lower dose of the immunoconjugate than would be required for unconjugated IL-2. Moreover, since application of IL-2 in form of immunoconjugates allows lower doses of the cytokine itself, the potential for undesirable side effects of IL-2 is restricted, and targeting the IL-2 to a specific site in the body by means of an immunoconjugate may also result in a reduction of systemic exposure and thus less side effects than obtained with unconjugated IL-2. In addition, the increased circulating half-life of an immunoconjugate compared to unconjugated IL-2 contributes to the efficacy of the immunoconjugate. However, this characteristic of IL-2 immunoconjugates may again aggravate potential side effects of the IL-2 molecule: Because of the significantly longer circulating half-life of IL-2 immunoconjugate in the bloodstream relative to unconjugated IL-2, the probability for IL-2 or other portions of the fusion protein molecule to activate components generally present in the vasculature is increased. The same concern applies to other fusion proteins that contain IL-2 fused to another moiety such as Fc or albumin, resulting in an extended half-life of IL-2 in the circulation. Therefore an immunoconjugate comprising a mutant IL-2 polypeptide as described herein and in WO 2012/107417, with reduced toxicity compared to wild-type forms of IL-2, is particularly advantageous.

As described hereinabove, targeting IL-2 directly to immune effector cells rather than tumor cells may be advantageous for IL-2 immunotherapy.

Accordingly, the invention provides a mutant IL-2 polypeptide as described hereinbefore, and an antibody that binds to PD-1. In one embodiment the mutant IL-2 polypeptide and the antibody form a fusion protein, i.e. the mutant IL-2 polypeptide shares a peptide bond with the antibody.

In some embodiments, the antibody comprises an Fc domain composed of a first and a second subunit. In a specific embodiment the mutant IL-2 polypeptide is fused at its amino-terminal amino acid to the carboxy-terminal amino acid of one of the subunits of the Fc domain, optionally through a linker peptide. In some embodiments, the antibody is a full-length antibody. In some embodiments, the antibody is an immunoglobulin molecule, particularly an IgG class immunoglobulin molecule, more particularly an IgG₁ subclass immunoglobulin molecule. In one such embodiment, the mutant IL-2 polypeptide shares an amino-terminal peptide bond with one of the immunoglobulin heavy chains. In certain embodiments the antibody is an antibody fragment. In some embodiments the antibody is a Fab molecule or a scFv molecule. In one embodiment the antibody is a Fab molecule. In another embodiment the antibody is a scFv molecule. The immunoconjugate may also comprise more than one antibody. Where more than one antibody is comprised in the immunoconjugate, e.g. a first and a second antibody, each antibody can be independently selected from various forms of antibodies and antibody fragments.

For example, the first antibody can be a Fab molecule and the second antibody can be a scFv molecule. In a specific embodiment each of said first and said second antibodies is a scFv molecule or each of said first and said second antibodies is a Fab molecule. In a particular embodiment each of said first and said second antibodies is a Fab molecule. In one embodiment each of said first and said second antibodies binds to PD-1.

### Immunoconjugate Formats

Exemplary immunoconjugate formats are described in PCT publication no. WO 2011/020783, which is incorporated herein by reference in its entirety. These immunoconjugates comprise at least two antibodies. Thus, in one embodiment, the immunoconjugate according to the present invention comprises a mutant IL-2 polypeptide as described herein, and at least a first and a second antibody. In a particular embodiment, said first and second antibody are independently selected from the group consisting of an Fv molecule, particularly a scFv molecule, and a Fab molecule. In a specific embodiment, said mutant IL-2 polypeptide shares an amino- or carboxy-terminal peptide bond with said first antibody and said second antibody shares an amino- or carboxy-terminal peptide bond with either i) the mutant IL-2 polypeptide or ii) the first antibody. In a particular embodiment, the immunoconjugate consists essentially of a mutant IL-2 polypeptide and first and second antibodies, particularly Fab molecules, joined by one or more linker sequences. Such formats have the advantage that they bind with high affinity to the target antigen (PD-1), but provide only monomeric binding to the IL-2 receptor, thus avoiding targeting the immunoconjugate to IL-2 receptor bearing immune cells at other locations than the target site. In a particular embodiment, a mutant IL-2 polypeptide shares a carboxy-terminal peptide bond with a first antibody, particularly a first Fab molecule, and further shares an amino-terminal peptide bond with a second antibody, particularly a second Fab molecule. In another embodiment, a first antibody, particularly a first Fab molecule, shares a carboxy-terminal peptide bond with a mutant IL-2 polypeptide, and further shares an amino-terminal peptide bond with a second antibody, particularly a second Fab molecule. In another embodiment, a first antibody, particularly a first Fab molecule, shares an amino-terminal peptide bond with a first mutant IL-2 polypeptide, and further shares a carboxy-terminal peptide with a second antibody, particularly a second Fab molecule. In a particular embodiment, a mutant IL-2 polypeptide shares a carboxy-terminal peptide bond with a first heavy chain variable region and further shares an amino-terminal peptide bond with a second heavy chain variable region. In another embodiment a mutant IL-2 polypeptide shares a carboxy-terminal peptide bond with a first light chain variable region and further shares an amino-terminal peptide bond with a second light chain variable region. In another embodiment, a first heavy or light chain variable region is joined by a carboxy-terminal peptide bond to a mutant IL-2 polypeptide and is further joined by an amino-terminal peptide bond to a second heavy or light chain variable region. In another embodiment, a first heavy or light chain variable region is joined by an amino-terminal peptide bond to a mutant IL-2 polypeptide and is further joined by a carboxy-terminal peptide bond to a second heavy or light chain variable region. In one embodiment, a mutant IL-2 polypeptide shares a carboxy-terminal peptide bond with a first Fab heavy or light chain and further shares an amino-terminal peptide bond with a second Fab heavy or light chain. In another embodiment, a first Fab heavy or light chain shares a carboxy-terminal peptide bond with a mutant IL-2 polypeptide and further shares an amino-terminal peptide bond with a second Fab heavy or light chain. In other embodiments, a first Fab heavy or light chain shares an amino-terminal peptide bond with a mutant IL-2 polypeptide and further shares a carboxy-terminal peptide bond with a second Fab heavy or light chain. In one embodiment, the immunoconjugate comprises a mutant IL-2 polypeptide sharing an amino-terminal peptide bond with one or more scFv molecules and further sharing a carboxy-terminal peptide bond with one or more scFv molecules.

Particularly suitable formats for the immunoconjugates according to the present invention, however comprise an immunoglobulin molecule as antibody. Such immunoconjugate formats are described in WO 2012/146628, which is incorporated herein by reference in its entirety.

Accordingly, in particular embodiments, the immunoconjugate comprises a mutant IL-2 polypeptide as described herein and an immunoglobulin molecule that binds to PD-1, particularly an IgG molecule, more particularly an IgG₁ molecule. In one embodiment the immunoconjugate comprises not more than one mutant IL-2 polypeptide. In one embodiment the immunoglobulin molecule is human. In one embodiment, the immunoglobulin molecule comprises a human constant region, e.g. a human CH1, CH2, CH3 and/or CL domain. In one embodiment, the immunoglobulin comprises a human Fc domain, particularly a human IgG₁ Fc domain. In one embodiment the mutant IL-2 polypeptide shares an amino- or carboxy-terminal peptide bond with the immunoglobulin molecule. In one embodiment, the immunoconjugate essentially consists of a mutant IL-2 polypeptide and an immunoglobulin molecule, particularly an IgG molecule, more particularly an IgG₁ molecule, joined by one or more linker sequences. In a specific embodiment the mutant IL-2 polypeptide is fused at its amino-terminal amino acid to the carboxy-terminal amino acid of one of the immunoglobulin heavy chains, optionally through a linker peptide.

The mutant IL-2 polypeptide may be fused to the antibody directly or through a linker peptide, comprising one or more amino acids, typically about 2-20 amino acids. Linker peptides are known in the art and are described herein. Suitable, non-immunogenic linker peptides include, for example, (G₄S)ₙ, (SG4)ₙ, (G₄S)ₙ or G₄(SG4)ₙ linker peptides. “n” is generally an integer from 1 to 10, typically from 2 to 4. In one embodiment the linker peptide has a length of at least 5 amino acids, in one embodiment a length of 5 to 100, in a further embodiment of 10 to 50 amino acids. In a particular embodiment, the linker peptide has a length of 15 amino acids. In one embodiment the linker peptide is (GxS)ₙ or (GxS)ₙGₘ with G=glycine, S=serine, and (x=3, n=3, 4, 5 or 6, and m=0, 1, 2 or 3) or (x=4, n=2, 3, 4 or 5 and m=0, 1, 2 or 3), in one embodiment x=4 and n=2 or 3, in a further embodiment x=4 and n=3. In a particular embodiment the linker peptide is (G4S)₃ (SEQ ID NO: 93). In one embodiment, the linker peptide has (or consists of) the amino acid sequence of SEQ ID NO: 93.

In a particular embodiment, the immunoconjugate comprises a mutant IL-2 molecule and an immunoglobulin molecule, particularly an IgG₁ subclass immunoglobulin molecule, that binds to PD-1, wherein the mutant IL-2 molecule is fused at its amino-terminal amino acid to the carboxy-terminal amino acid of one of the immunoglobulin heavy chains through the linker peptide of SEQ ID NO: 93.

In a particular embodiment, the immunoconjugate comprises a mutant IL-2 molecule and an antibody that binds to PD-1, wherein the antibody comprises an Fc domain, particularly a human IgG₁ Fc domain, composed of a first and a second subunit, and the mutant IL-2 molecule is fused at its amino-terminal amino acid to the carboxy-terminal amino acid of one of the subunits of the Fc domain through the linker peptide of SEQ ID NO: 93.

### PD-1 Antibodies

The antibody comprised in the immunoconjugate of the invention binds to PD-1, particularly human PD-1, and is able to direct the mutant IL-2 polypeptide to a target site where PD-1 is expressed, particularly to a T cell that expresses PD-1, for example associated with a tumor.

Suitable PD-1 antibodies that may be used in the immunoconjugate of the invention are described in PCT patent application no. PCT/EP2016/073248, which is incorporated herein by reference in its entirety.

The immunoconjugate of the invention may comprise two or more antibodies, which may bind to the same or to different antigens. In particular embodiments, however, each of these antibodies binds to PD-1. In one embodiment, the antibody comprised in the immunoconjugate of the invention is monospecific. In a particular embodiment, the immunoconjugate comprises a single, monospecific antibody, particularly a monospecific immunoglobulin molecule.

The antibody can be any type of antibody or fragment thereof that retains specific binding to PD-1, particularly human PD-1. Antibody fragments include, but are not limited to, Fv molecules, scFv molecule, Fab molecule, and F(ab′)2 molecules. In particular embodiments, however, the antibody is a full-length antibody. In some embodiments, the antibody comprises an Fc domain, composed of a first and a second subunit. In some embodiments, the antibody is an immunoglobulin, particularly an IgG class, more particularly an IgG₁ subclass immunoglobulin.

In some embodiments, the antibody is a monoclonal antibody.

In some embodiments, the antibody comprises a CDR-H1 comprising the amino acid sequence of SEQ ID NO:74, a CDR-H2 comprising the amino acid sequence of SEQ ID NO:75, a CDR-H3 comprising the amino acid sequence of SEQ ID NO:76, a CDR-L1 comprising the amino acid sequence of SEQ ID NO:77, a HVR-L2 comprising the amino acid sequence of SEQ ID NO:78, and a CDR-L3 comprising the amino acid sequence of SEQ ID NO:79.

In some embodiments, the antibody comprises (a) a heavy chain variable region (VH) comprising a CDR-H1 comprising the amino acid sequence of SEQ ID NO:74, a CDR-H2 comprising the amino acid sequence of SEQ ID NO:75, a CDR-H3 comprising the amino acid sequence of SEQ ID NO:76, and (b) a light chain variable region (VL) comprising a CDR-L1 comprising the amino acid sequence of SEQ ID NO:77, a CDR-L2 comprising the amino acid sequence of SEQ ID NO:78, and a CDR-L3 comprising the amino acid sequence of SEQ ID NO:79. In some embodiments, the heavy and/or light chain variable region is a humanized variable region. In some embodiments, the heavy and/or light chain variable region comprises human framework regions (FR).

In some embodiments, the antibody comprises a heavy chain variable region (VH) comprising an amino acid sequence that is at least about 95%, 96%, 97%, 98%, 99% or 100% identical to the amino acid sequence of SEQ ID NO:80. In some embodiments, the antibody comprises a light chain variable region (VL) comprising an amino acid sequence that is at least about 95%, 96%, 97%, 98%, 99% or 100% identical to an amino acid sequence of SEQ ID NO:81. In a particular embodiment, the antibody comprises (a) a heavy chain variable region (VH) comprising the amino acid sequence of SEQ ID NO:80, and (b) a light chain variable region (VL) comprising the amino acid sequence of SEQ ID NO:81.

In some embodiments, the antibody is a humanized antibody. In one embodiment, the antibody is an immunoglobulin molecule comprising a human constant region, particularly an IgG class immunoglobulin molecule comprising a human CH1, CH2, CH3 and/or CL domain.

### Fc Domain

In particular embodiments, the antibody comprised in the immunoconjugates according to the invention comprises an Fc domain, composed of a first and a second subunit. The Fc domain of an antibody consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other. In one embodiment the immunoconjugate of the invention comprises not more than one Fc domain. In one embodiment the Fc domain of the antibody comprised in the immunoconjugate is an IgG Fc domain. In a particular embodiment the Fc domain is an IgG₁ Fc domain. In another embodiment the Fc domain is an IgG₄ Fc domain. In a more specific embodiment, the Fc domain is an IgG₄ Fc domain comprising an amino acid substitution at position S228 (Kabat EU index numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In a further particular embodiment the Fc domain is a human Fc domain. In an even more particular embodiment, the Fc domain is a human IgG₁ Fc domain.

### Fc Domain Modifications Promoting Heterodimerization

Immunoconjugates according to the invention comprise a mutant IL-2 polypeptide, particularly a single (not more than one) mutant IL-2 polypeptide, fused to one or the other of the two subunits of the Fc domain, thus the two subunits of the Fc domain are typically comprised in two non-identical polypeptide chains. Recombinant co-expression of these polypeptides and subsequent dimerization leads to several possible combinations of the two polypeptides. To improve the yield and purity of the immunoconjugate in recombinant production, it will thus be advantageous to introduce in the Fc domain of the antibody a modification promoting the association of the desired polypeptides.

Accordingly, in particular embodiments, the Fc domain of the antibody comprised in the immunoconjugate according to the invention comprises a modification promoting the association of the first and the second subunit of the Fc domain. The site of most extensive protein-protein interaction between the two subunits of a human IgG Fc domain is in the CH3 domain of the Fc domain. Thus, in one embodiment said modification is in the CH3 domain of the Fc domain.

There exist several approaches for modifications in the CH3 domain of the Fc domain in order to enforce heterodimerization, which are well described e.g. in WO 96/27011, WO 98/050431, EP 1870459, WO 2007/110205, WO 2007/147901, WO 2009/089004, WO 2010/129304, WO 2011/90754, WO 2011/143545, WO 2012058768, WO 2013157954, WO 2013096291. Typically, in all such approaches the CH3 domain of the first subunit of the Fc domain and the CH3 domain of the second subunit of the Fc domain are both engineered in a complementary manner so that each CH3 domain (or the heavy chain comprising it) can no longer homodimerize with itself but is forced to heterodimerize with the complementarily engineered other CH3 domain (so that the first and second CH3 domain heterodimerize and no homodimers between the two first or the two second CH3 domains are formed).

In a specific embodiment said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan).

Compensatory cavities of identical or similar size to the protuberances are created in the interface of the second polypeptide by replacing large amino acid side chains with smaller ones (e.g. alanine or threonine).

Accordingly, in a particular embodiment, in the CH3 domain of the first subunit of the Fc domain of the antibody comprised in the immunoconjugate an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

Preferably said amino acid residue having a larger side chain volume is selected from the group consisting of arginine (R), phenylalanine (F), tyrosine (Y), and tryptophan (W).

Preferably said amino acid residue having a smaller side chain volume is selected from the group consisting of alanine (A), serine (S), threonine (T), and valine (V).

The protuberance and cavity can be made by altering the nucleic acid encoding the polypeptides, e.g. by site-specific mutagenesis, or by peptide synthesis.

In a specific embodiment, in the CH3 domain of the first subunit of the Fc domain (the “knobs” subunit) the threonine residue at position 366 is replaced with a tryptophan residue (T366W), and in the CH3 domain of the second subunit of the Fc domain (the “hole” subunit) the tyrosine residue at position 407 is replaced with a valine residue (Y407V). In one embodiment, in the second subunit of the Fc domain additionally the threonine residue at position 366 is replaced with a serine residue (T366S) and the leucine residue at position 368 is replaced with an alanine residue (L368A) (numberings according to Kabat EU index).

In yet a further embodiment, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numberings according to Kabat EU index). Introduction of these two cysteine residues results in formation of a disulfide bridge between the two subunits of the Fc domain, further stabilizing the dimer (Carter, J Immunol Methods 248, 7-15 (2001)).

In a particular embodiment, the first subunit of the Fc domain comprises the amino acid substitutions S354C and T366W, and the second subunit of the Fc domain comprises the amino acid substitutions Y349C, T366S, L368A and Y407V (numbering according to Kabat EU index). In some embodiments, the second subunit of the Fc domain additionally comprises the amino acid substitutions H435R and Y436F (numbering according to Kabat EU index).

In a particular embodiment the mutant IL-2 polypeptide is fused (optionally through a linker peptide) to the first subunit of the Fc domain (comprising the “knob” modification). Without wishing to be bound by theory, fusion of the mutant IL-2 polypeptide to the knob-containing subunit of the Fc domain will (further) minimize the generation of immunoconjugates comprising two mutant IL-2 polypeptides (steric clash of two knob-containing polypeptides).

Other techniques of CH3-modification for enforcing the heterodimerization are contemplated as alternatives according to the invention and are described e.g. in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291. In one embodiment the heterodimerization approach described in EP 1870459, is used alternatively. This approach is based on the introduction of charged amino acids with opposite charges at specific amino acid positions in the CH3/CH3 domain interface between the two subunits of the Fc domain. One preferred embodiment for the antibody comprised in the immunoconjugate of the invention are amino acid mutations R409D; K370E in one of the two CH3 domains (of the Fc domain) and amino acid mutations D399K; E357K in the other one of the CH3 domains of the Fc domain (numbering according to Kabat EU index).

In another embodiment, the antibody comprised in the immunoconjugate of the invention comprises amino acid mutation T366W in the CH3 domain of the first subunit of the Fc domain and amino acid mutations T366S, L368A, Y407V in the CH3 domain of the second subunit of the Fc domain, and additionally amino acid mutations R409D; K370E in the CH3 domain of the first subunit of the Fc domain and amino acid mutations D399K; E357K in the CH3 domain of the second subunit of the Fc domain (numberings according to Kabat EU index).

In another embodiment, the antibody comprised in the immunoconjugate of the invention comprises amino acid mutations S354C, T366W in the CH3 domain of the first subunit of the Fc domain and amino acid mutations Y349C, T366S, L368A, Y407V in the CH3 domain of the second subunit of the Fc domain, or said antibody comprises amino acid mutations Y349C, T366W in the CH3 domain of the first subunit of the Fc domain and amino acid mutations S354C, T366S, L368A, Y407V in the CH3 domains of the second subunit of the Fc domain and additionally amino acid mutations R409D; K370E in the CH3 domain of the first subunit of the Fc domain and amino acid mutations D399K; E357K in the CH3 domain of the second subunit of the Fc domain (all numberings according to Kabat EU index).

In one embodiment, the heterodimerization approach described in WO 2013/157953 is used alternatively. In one embodiment, a first CH3 domain comprises amino acid mutation T366K and a second CH3 domain comprises amino acid mutation L351D (numberings according to Kabat EU index). In a further embodiment, the first CH3 domain comprises further amino acid mutation L351K. In a further embodiment, the second CH3 domain comprises further an amino acid mutation selected from Y349E, Y349D and L368E (preferably L368E) (numberings according to Kabat EU index).

In one embodiment, the heterodimerization approach described in WO 2012/058768 is used alternatively. In one embodiment, a first CH3 domain comprises amino acid mutations L351Y, Y407A and a second CH3 domain comprises amino acid mutations T366A, K409F. In a further embodiment, the second CH3 domain comprises a further amino acid mutation at position T411, D399, S400, F405, N390, or K392, e.g. selected from a) T411N, T411R, T411Q, T411K, T411D, T411E or T411W, b) D399R, D399W, D399Y or D399K, c) S400E, S400D, S400R, or S400K, d) F405I, F405M, F405T, F405S, F405V or F405W, e) N390R, N390K or N390D, f) K392V, K392M, K392R, K392L, K392F or K392E (numberings according to Kabat EU index).

In a further embodiment, a first CH3 domain comprises amino acid mutations L351Y, Y407A and a second CH3 domain comprises amino acid mutations T366V, K409F. In a further embodiment a first CH3 domain comprises amino acid mutation Y407A and a second CH3 domain comprises amino acid mutations T366A, K409F. In a further embodiment, the second CH3 domain further comprises amino acid mutations K392E, T411E, D399R and S400R (numberings according to Kabat EU index).

In one embodiment the heterodimerization approach described in WO 2011/143545 is used alternatively, e.g. with the amino acid modification at a position selected from the group consisting of 368 and 409 (numbering according to Kabat EU index).

In one embodiment, the heterodimerization approach described in WO 2011/090762, which also uses the knobs-into-holes technology described above, is used alternatively. In one embodiment, a first CH3 domain comprises amino acid mutation T366W and a second CH3 domain comprises amino acid mutation Y407A. In one embodiment, a first CH3 domain comprises amino acid mutation T366Y and a second CH3 domain comprises amino acid mutation Y407T (numberings according to Kabat EU index).

In one embodiment, the antibody comprised in the immunoconjugate or its Fc domain is of IgG₂ subclass and the heterodimerization approach described in WO 2010/129304 is used alternatively.

In an alternative embodiment, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable. In one such embodiment, a first CH3 domain comprises amino acid substitution of K392 or N392 with a negatively charged amino acid (e.g. glutamic acid (E), or aspartic acid (D), preferably K392D or N392D) and a second CH3 domain comprises amino acid substitution of D399, E356, D356, or E357 with a positively charged amino acid (e.g. lysine (K) or arginine (R), preferably D399K, E356K, D356K, or E357K, and more preferably D399K and E356K). In a further embodiment, the first CH3 domain further comprises amino acid substitution of K409 or R409 with a negatively charged amino acid (e.g. glutamic acid (E), or aspartic acid (D), preferably K409D or R409D). In a further embodiment, the first CH3 domain further or alternatively comprises amino acid substitution of K439 and/or K370 with a negatively charged amino acid (e.g. glutamic acid (E), or aspartic acid (D)) (all numberings according to Kabat EU index).

In yet a further embodiment, the heterodimerization approach described in WO 2007/147901 is used alternatively. In one embodiment, a first CH3 domain comprises amino acid mutations K253E, D282K, and K322D and a second CH3 domain comprises amino acid mutations D239K, E240K, and K292D (numberings according to Kabat EU index).

In still another embodiment, the heterodimerization approach described in WO 2007/110205 can be used alternatively.

In one embodiment, the first subunit of the Fc domain comprises amino acid substitutions K392D and K409D, and the second subunit of the Fc domain comprises amino acid substitutions D356K and D399K (numbering according to Kabat EU index).

Fc Domain Modifications Reducing Fc Receptor Binding and/or Effector Function

The Fc domain confers to the immunoconjugate favorable pharmacokinetic properties, including a long serum half-life which contributes to good accumulation in the target tissue and a favorable tissue-blood distribution ratio. At the same time it may, however, lead to undesirable targeting of the immunoconjugate to cells expressing Fc receptors rather than to the preferred antigen-bearing cells. Moreover, the co-activation of Fc receptor signaling pathways may lead to cytokine release which, in combination with the IL-2 polypeptide and the long half-life of the immunoconjugate, results in excessive activation of cytokine receptors and severe side effects upon systemic administration. In line with this, conventional IgG-IL-2 immunoconjugates have been described to be associated with infusion reactions (see e.g. King et al., J Clin Oncol 22, 4463-4473 (2004)).

Accordingly, in particular embodiments, the Fc domain of the antibody comprised in the immunoconjugate according to the invention exhibits reduced binding affinity to an Fc receptor and/or reduced effector function, as compared to a native IgG₁ Fc domain. In one such embodiment the Fc domain (or the antibody comprising said Fc domain) exhibits less than 50%, preferably less than 20%, more preferably less than 10% and most preferably less than 5% of the binding affinity to an Fc receptor, as compared to a native IgG₁ Fc domain (or an antibody comprising a native IgG₁ Fc domain), and/or less than 50%, preferably less than 20%, more preferably less than 10% and most preferably less than 5% of the effector function, as compared to a native IgG₁ Fc domain domain (or an antibody comprising a native IgG₁ Fc domain). In one embodiment, the Fc domain domain (or an antibody comprising said Fc domain) does not substantially bind to an Fc receptor and/or induce effector function. In a particular embodiment the Fc receptor is an Fey receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of CDC, ADCC, ADCP, and cytokine secretion. In a particular embodiment the effector function is ADCC. In one embodiment the Fc domain domain exhibits substantially similar binding affinity to neonatal Fc receptor (FcRn), as compared to a native IgG₁ Fc domain domain. Substantially similar binding to FcRn is achieved when the Fc domain (or an antibody comprising said Fc domain) exhibits greater than about 70%, particularly greater than about 80%, more particularly greater than about 90% of the binding affinity of a native IgG₁ Fc domain (or an antibody comprising a native IgG₁ Fc domain) to FcRn.

In certain embodiments the Fc domain is engineered to have reduced binding affinity to an Fc receptor and/or reduced effector function, as compared to a non-engineered Fc domain. In particular embodiments, the Fc domain of the antibody comprised in the immunoconjugate comprises one or more amino acid mutation that reduces the binding affinity of the Fc domain to an Fc receptor and/or effector function. Typically, the same one or more amino acid mutation is present in each of the two subunits of the Fc domain. In one embodiment the amino acid mutation reduces the binding affinity of the Fc domain to an Fc receptor. In one embodiment the amino acid mutation reduces the binding affinity of the Fc domain to an Fc receptor by at least 2-fold, at least 5-fold, or at least 10-fold. In embodiments where there is more than one amino acid mutation that reduces the binding affinity of the Fc domain to the Fc receptor, the combination of these amino acid mutations may reduce the binding affinity of the Fc domain to an Fc receptor by at least 10-fold, at least 20-fold, or even at least 50-fold. In one embodiment the antibody comprising an engineered Fc domain exhibits less than 20%, particularly less than 10%, more particularly less than 5% of the binding affinity to an Fc receptor as compared to an antibody comprising a non-engineered Fc domain. In a particular embodiment the Fc receptor is an Fcγ receptor. In some embodiments the Fc receptor is a human Fc receptor. In some embodiments the Fc receptor is an activating Fc receptor. In a specific embodiment the Fc receptor is an activating human Fey receptor, more specifically human FcγRIIIa, FcγRI or FcγRIIa, most specifically human FcγRIIIa. Preferably, binding to each of these receptors is reduced. In some embodiments binding affinity to a complement component, specifically binding affinity to C1q, is also reduced. In one embodiment binding affinity to neonatal Fc receptor (FcRn) is not reduced. Substantially similar binding to FcRn, i.e. preservation of the binding affinity of the Fc domain to said receptor, is achieved when the Fc domain (or an antibody comprising said Fc domain) exhibits greater than about 70% of the binding affinity of a non-engineered form of the Fc domain (or an antibody comprising said non-engineered form of the Fc domain) to FcRn. The Fc domain, or antibody comprised in the immunoconjugate of the invention comprising said Fc domain, may exhibit greater than about 80% and even greater than about 90% of such affinity. In certain embodiments the Fc domain of the antibody comprised in the immunoconjugate is engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced crosslinking of target-bound antibodies, reduced dendritic cell maturation, or reduced T cell priming. In one embodiment the reduced effector function is one or more selected from the group of reduced CDC, reduced ADCC, reduced ADCP, and reduced cytokine secretion. In a particular embodiment the reduced effector function is reduced ADCC. In one embodiment the reduced ADCC is less than 20% of the ADCC induced by a non-engineered Fc domain (or an antibody comprising a non-engineered Fc domain).

In one embodiment the amino acid mutation that reduces the binding affinity of the Fc domain to an Fc receptor and/or effector function is an amino acid substitution. In one embodiment the Fc domain comprises an amino acid substitution at a position selected from the group of E233, L234, L235, N297, P331 and P329 (numberings according to Kabat EU index). In a more specific embodiment the Fc domain comprises an amino acid substitution at a position selected from the group of L234, L235 and P329 (numberings according to Kabat EU index). In some embodiments the Fc domain comprises the amino acid substitutions L234A and L235A (numberings according to Kabat EU index). In one such embodiment, the Fc domain is an IgG₁ Fc domain, particularly a human IgG₁ Fc domain. In one embodiment the Fc domain comprises an amino acid substitution at position P329. In a more specific embodiment the amino acid substitution is P329A or P329G, particularly P329G (numberings according to Kabat EU index). In one embodiment the Fc domain comprises an amino acid substitution at position P329 and a further amino acid substitution at a position selected from E233, L234, L235, N297 and P331 (numberings according to Kabat EU index). In a more specific embodiment the further amino acid substitution is E233P, L234A, L235A, L235E, N297A, N297D or P331S. In particular embodiments the Fc domain comprises amino acid substitutions at positions P329, L234 and L235 (numberings according to Kabat EU index). In more particular embodiments the Fc domain comprises the amino acid mutations L234A, L235A and P329G (“P329G LALA”, “PGLALA” or “LALAPG”). Specifically, in particular embodiments, each subunit of the Fc domain comprises the amino acid substitutions L234A, L235A and P329G (Kabat EU index numbering), i.e. in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index). In one such embodiment, the Fc domain is an IgG₁ Fc domain, particularly a human IgG₁ Fc domain. The “P329G LALA” combination of amino acid substitutions almost completely abolishes Fey receptor (as well as complement) binding of a human IgG₁ Fc domain, as described in PCT publication no. WO 2012/130831, which is incorporated herein by reference in its entirety. WO 2012/130831 also describes methods of preparing such mutant Fc domains and methods for determining its properties such as Fc receptor binding or effector functions.

IgG₄ antibodies exhibit reduced binding affinity to Fe receptors and reduced effector functions as compared to IgG₁ antibodies. Hence, in some embodiments the Fc domain of the antibody comprised in the immunoconjugate of the invention is an IgG₄ Fc domain, particularly a human IgG₄ Fc domain. In one embodiment the IgG₄ Fc domain comprises amino acid substitutions at position S228, specifically the amino acid substitution S228P (numberings according to Kabat EU index). To further reduce its binding affinity to an Fc receptor and/or its effector function, in one embodiment the IgG₄ Fc domain comprises an amino acid substitution at position L235, specifically the amino acid substitution L235E (numberings according to Kabat EU index). In another embodiment, the IgG₄ Fc domain comprises an amino acid substitution at position P329, specifically the amino acid substitution P329G (numberings according to Kabat EU index). In a particular embodiment, the IgG₄ Fc domain comprises amino acid substitutions at positions S228, L235 and P329, specifically amino acid substitutions S228P, L235E and P329G (numberings according to Kabat EU index). Such IgG₄ Fc domain mutants and their Fcγ receptor binding properties are described in PCT publication no. WO 2012/130831, incorporated herein by reference in its entirety.

In a particular embodiment, the Fc domain exhibiting reduced binding affinity to an Fc receptor and/or reduced effector function, as compared to a native IgG₁ Fc domain, is a human IgG₁ Fc domain comprising the amino acid substitutions L234A, L235A and optionally P329G, or a human IgG₄ Fc domain comprising the amino acid substitutions S228P, L235E and optionally P329G (numberings according to Kabat EU index).

In certain embodiments N-glycosylation of the Fc domain has been eliminated. In one such embodiment, the Fc domain comprises an amino acid mutation at position N297, particularly an amino acid substitution replacing asparagine by alanine (N297A) or aspartic acid (N297D) (numberings according to Kabat EU index).

In addition to the Fc domains described hereinabove and in PCT publication no. WO 2012/130831, Fc domains with reduced Fc receptor binding and/or effector function also include those with substitution of one or more of Fc domain residues 238, 265, 269, 270, 297, 327 and 329 (U.S. Pat. No. 6,737,056) (numberings according to Kabat EU index). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (Cytiva), and Fc receptors such as may be obtained by recombinant expression. Alternatively, binding affinity of Fc domains or antibodies comprising an Fc domain for Fc receptors may be evaluated using cell lines known to express particular Fc receptors, such as human NK cells expressing Fcγ IIIa receptor.

Effector function of an Fc domain, or an antibody comprising an Fc domain, can be measured by methods known in the art. Examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, CA); and CytoTox 96® non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998).

In some embodiments, binding of the Fc domain to a complement component, specifically to C1q, is reduced. Accordingly, in some embodiments wherein the Fc domain is engineered to have reduced effector function, said reduced effector function includes reduced CDC. C1q binding assays may be carried out to determine whether the Fc domain, or antibody comprising the Fc domain, is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

FcRn binding and in vivo clearance/half life determinations can also be performed using methods known in the art (see, e.g., Petkova, S. B. et al., Int'l. Immunol. 18(12):1759-1769 (2006); WO 2013/120929).

### Particular Aspects of the Invention

In one aspect, the invention provides an immunoconjugate comprising a mutant IL-2 polypeptide and an antibody that binds to PD-1, wherein the mutant IL-2 polypeptide is a human IL-2 molecule comprising the amino acid substitutions F42A, Y45A, L72G and Q126T (numbering relative to the human IL-2 sequence SEQ ID NO: 90); and wherein the antibody comprises (a) a heavy chain variable region (VH) comprising the amino acid sequence of SEQ ID NO:80, and (b) a light chain variable region (VL) comprising the amino acid sequence of SEQ ID NO:81. In one aspect, the invention provides an immunoconjugate comprising a mutant IL-2 polypeptide and an antibody that binds to PD-1, wherein the mutant IL-2 polypeptide is a human IL-2 molecule comprising the amino acid substitutions T3A, F42A, Y45A, L72G, C125A and Q126T (numbering relative to the human IL-2 sequence SEQ ID NO: 90); and wherein the antibody comprises (a) a heavy chain variable region (VH) comprising the amino acid sequence of SEQ ID NO:80, and (b) a light chain variable region (VL) comprising the amino acid sequence of SEQ ID NO:81. In one aspect, the invention provides an immunoconjugate comprising a mutant IL-2 polypeptide and an antibody that binds to PD-1, wherein the mutant IL-2 polypeptide comprises the amino acid sequence of SEQ ID NO: 92; and wherein the antibody comprises (a) a heavy chain variable region (VH) comprising the amino acid sequence of SEQ ID NO:80, and (b) a light chain variable region (VL) comprising the amino acid sequence of SEQ ID NO:81. In one embodiment according to any of the above aspects of the invention, the antibody is an IgG class immunoglobulin, comprising a human IgG₁ Fc domain composed of a first and a second subunit, wherein in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numberings according to Kabat EU index), and wherein further each subunit of the Fc domain comprises the amino acid substitutions L234A, L235A and P329G (Kabat EU index numbering). In this embodiment, the mutant IL-2 polypeptide may be fused at its amino-terminal amino acid to the carboxy-terminal amino acid of the first subunit of the Fc domain, through a linker peptide of SEQ ID NO: 93. In one aspect, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO:21, a polypeptide comprising an amino acid sequence that is at least about 80%, 85%, 90%, 95%, 96%, 97%, 98%, 99% or 100% identical to the sequence of SEQ ID NO:22, and a polypeptide comprising an amino acid sequence that is at least about 80%, 85%, 90%, 95%, 96%, 97%, 98%, 99% or 100% identical to the sequence of SEQ ID NO:35.

In one aspect, the invention provides an immunoconjugate comprising a mutant IL-2 polypeptide and an antibody that binds to PD-1, wherein the mutant IL-2 polypeptide is a human IL-2 molecule comprising the amino acid substitutions F42A, Y45A, L72G and N88D (numbering relative to the human IL-2 sequence SEQ ID NO: 90); and wherein the antibody comprises (a) a heavy chain variable region (VH) comprising the amino acid sequence of SEQ ID NO:80, and (b) a light chain variable region (VL) comprising the amino acid sequence of SEQ ID NO:81.

In one aspect, the invention provides an immunoconjugate comprising a mutant IL-2 polypeptide and an antibody that binds to PD-1, wherein the mutant IL-2 polypeptide is a human IL-2 molecule comprising the amino acid substitutions T3A, F42A, Y45A, L72G, N88D and C125A (numbering relative to the human IL-2 sequence SEQ ID NO: 90); and wherein the antibody comprises (a) a heavy chain variable region (VH) comprising the amino acid sequence of SEQ ID NO:80, and (b) a light chain variable region (VL) comprising the amino acid sequence of SEQ ID NO:81. In one aspect, the invention provides an immunoconjugate comprising a mutant IL-2 polypeptide and an antibody that binds to PD-1, wherein the mutant IL-2 polypeptide comprises the amino acid sequence of SEQ ID NO: 98; and wherein the antibody comprises (a) a heavy chain variable region (VH) comprising the amino acid sequence of SEQ ID NO:80, and (b) a light chain variable region (VL) comprising the amino acid sequence of SEQ ID NO:81. In one embodiment according to any of the above aspects of the invention, the antibody is an IgG class immunoglobulin, comprising a human IgG₁ Fc domain composed of a first and a second subunit, wherein in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numberings according to Kabat EU index), and wherein further each subunit of the Fc domain comprises the amino acid substitutions L234A, L235A and P329G (Kabat EU index numbering). In this embodiment, the mutant IL-2 polypeptide may be fused at its amino-terminal amino acid to the carboxy-terminal amino acid of the first subunit of the Fc domain, through a linker peptide of SEQ ID NO: 93. In one aspect, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO:21, a polypeptide comprising an amino acid sequence that is at least about 80%, 85%, 90%, 95%, 96%, 97%, 98%, 99% or 100% identical to the sequence of SEQ ID NO:22, and a polypeptide comprising an amino acid sequence that is at least about 80%, 85%, 90%, 95%, 96%, 97%, 98%, 99% or 100% identical to the sequence of SEQ ID NO:100.

In one aspect, the invention provides an immunoconjugate comprising a mutant IL-2 polypeptide and an antibody that binds to PD-1, wherein the mutant IL-2 polypeptide is a human IL-2 molecule comprising the amino acid substitutions F42A, Y45A, L72G and N88Q (numbering relative to the human IL-2 sequence SEQ ID NO: 90); and wherein the antibody comprises (a) a heavy chain variable region (VH) comprising the amino acid sequence of SEQ ID NO:80, and (b) a light chain variable region (VL) comprising the amino acid sequence of SEQ ID NO:81. In one aspect, the invention provides an immunoconjugate comprising a mutant IL-2 polypeptide and an antibody that binds to PD-1, wherein the mutant IL-2 polypeptide is a human IL-2 molecule comprising the amino acid substitutions T3A, F42A, Y45A, L72G, N88Q and C125A (numbering relative to the human IL-2 sequence SEQ ID NO: 90); and wherein the antibody comprises (a) a heavy chain variable region (VH) comprising the amino acid sequence of SEQ ID NO:80, and (b) a light chain variable region (VL) comprising the amino acid sequence of SEQ ID NO:81. In one aspect, the invention provides an immunoconjugate comprising a mutant IL-2 polypeptide and an antibody that binds to PD-1, wherein the mutant IL-2 polypeptide comprises the amino acid sequence of SEQ ID NO: 99; and wherein the antibody comprises (a) a heavy chain variable region (VH) comprising the amino acid sequence of SEQ ID NO:80, and (b) a light chain variable region (VL) comprising the amino acid sequence of SEQ ID NO:81.

In one embodiment according to any of the above aspects of the invention, the antibody is an IgG class immunoglobulin, comprising a human IgG₁ Fc domain composed of a first and a second subunit, wherein in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numberings according to Kabat EU index), and wherein further each subunit of the Fc domain comprises the amino acid substitutions L234A, L235A and P329G (Kabat EU index numbering). In this embodiment, the mutant IL-2 polypeptide may be fused at its amino-terminal amino acid to the carboxy-terminal amino acid of the first subunit of the Fc domain, through a linker peptide of SEQ ID NO: 93. In one aspect, the invention provides an immunoconjugate comprising a polypeptide comprising an amino acid sequence that is at least about 80%, 85%, 90%, 95%, 96%, 97%, 98%, 99% or 100% identical to the sequence of SEQ ID NO:21, a polypeptide comprising an amino acid sequence that is at least about 80%, 85%, 90%, 95%, 96%, 97%, 98%, 99% or 100% identical to the sequence of SEQ ID NO:22, and a polypeptide comprising an amino acid sequence that is at least about 80%, 85%, 90%, 95%, 96%, 97%, 98%, 99% or 100% identical to the sequence of SEQ ID NO:31.

Bispecific Antigen Binding Molecules that Bind to PD-1 and LAG3

The immunoconjugate of the invention comprises a bispecific antigen binding molecule, i.e. an antigen binding molecule that comprises at least two antigen binding moieties capable of specific binding to two distinct antigenic determinants (such as PD-1 and LAG3).

The bispecific antigen binding molecule comprised in the immunoconjugate of the invention binds to PD-1 and LAG3, particularly human PD-1 and human LAG3, and is able to direct the mutant IL-2 polypeptide to a target site where PD-1 and/or LAG3 is expressed, particularly to a T cell that expresses PD-1 and/or LAG3, for example associated with a tumor.

According to particular embodiments of the invention, the antigen binding moieties comprised in the bispecific antigen binding molecule are Fab molecules (i.e. antigen binding domains composed of a heavy and a light chain, each comprising a variable and a constant domain). In one embodiment, the first and/or the second antigen binding moiety is a Fab molecule. In one embodiment, said Fab molecule is human. In a particular embodiment, said Fab molecule is humanized. In yet another embodiment, said Fab molecule comprises human heavy and light chain constant domains.

Preferably, at least one of the antigen binding moieties is a crossover Fab molecule. Such modification reduces mispairing of heavy and light chains from different Fab molecules, thereby improving the yield and purity of the bispecific antigen binding molecule in recombinant production. In a particular crossover Fab molecule useful for the bispecific antigen binding molecule comprised in the immunoconjugate of the invention, the variable domains of the Fab light chain and the Fab heavy chain (VL and VH, respectively) are exchanged. Even with this domain exchange, however, the preparation of the bispecific antigen binding molecule may comprise certain side products due to a so-called Bence Jones-type interaction between mispaired heavy and light chains (see Schaefer et al, PNAS, 108 (2011) 11187-11191). To further reduce mispairing of heavy and light chains from different Fab molecules and thus increase the purity and yield of the desired bispecific antigen binding molecule, charged amino acids with opposite charges may be introduced at specific amino acid positions in the CH1 and CL domains of either the Fab molecule binding to PD-1, or the Fab molecule binding to LAG3, as further described herein. Charge modifications are made either in the conventional Fab molecule comprised in the bispecific antigen binding molecule, or in the (VH/VL) crossover Fab molecule comprised in the bispecific antigen binding molecule (but not in both). In particular embodiments, the charge modifications are made in the conventional Fab molecule comprised in the bispecific antigen binding molecule (which in particular embodiments binds to LAG3).

### First Antigen Binding Moiety

The bispecific antigen binding molecule comprised in the immunoconjugate of the invention comprises at least one antigen binding moiety, particularly a Fab molecule, that binds to PD-1, particularly human PD-1 (first antigen). In particular embodiments, the antigen binding moiety that binds to PD-1 is a crossover Fab molecule as described herein, i.e. a Fab molecule wherein the variable domains VH and VL or the constant domains CH1 and CL of the Fab heavy and light chains are exchanged/replaced by each other. In such embodiments, the antigen binding moiety that binds to LAG3 is a conventional Fab molecule. In alternative embodiments, the antigen binding moiety which binds to LAG3 is a crossover Fab molecule as described herein, i.e. a Fab molecule wherein the variable domains VH and VL or the constant domains CH1 and CL of the Fab heavy and light chains are exchanged/replaced by each other. In such embodiments, the antigen binding moiety that binds to PD-1 is a conventional Fab molecule.

In some embodiments, the first antigen binding moiety comprises a CDR-H1 comprising the amino acid sequence of SEQ ID NO:74, a CDR-H2 comprising the amino acid sequence of SEQ ID NO:75, a CDR-H3 comprising the amino acid sequence of SEQ ID NO:76, a CDR-L1 comprising the amino acid sequence of SEQ ID NO:77, a CDR-L2 comprising the amino acid sequence of SEQ ID NO:78, and a CDR-L3 comprising the amino acid sequence of SEQ ID NO:79.

In some embodiments, the first antigen binding moiety comprises (a) a heavy chain variable region (VH) comprising a CDR-H1 comprising the amino acid sequence of SEQ ID NO:74, a CDR-H2 comprising the amino acid sequence of SEQ ID NO:75, and a CDR-H3 comprising the amino acid sequence of SEQ ID NO:76, and (b) a light chain variable region (VL) comprising a CDR-L1 comprising the amino acid sequence of SEQ ID NO:77, a CDR-L2 comprising the amino acid sequence of SEQ ID NO:78, and a CDR-L3 comprising the amino acid sequence of SEQ ID NO:79.

In some embodiments, the first antigen binding moiety is (derived from) a humanized antibody. In one embodiment, the VH is a humanized VH and/or the VL is a humanized VL. In one embodiment, the first antigen binding moiety comprises CDRs as in any of the above embodiments, and further comprises an acceptor human framework, e.g. a human immunoglobulin framework or a human consensus framework. In some embodiments, the heavy and/or light chain variable region comprises human framework regions (FR).

In some embodiments, the first antigen binding moiety comprises (a) a heavy chain variable region (VH) comprising an amino acid sequence that is at least about 95%, 96%, 97%, 98%, 99% or 100% identical to the amino acid sequence of SEQ ID NO:80. In some embodiments, the first antigen binding moiety comprises a light chain variable region (VL) comprising an amino acid sequence that is at least about 95%, 96%, 97%, 98%, 99% or 100% identical to the amino acid sequence of SEQ ID NO:81. In some embodiments, the first antigen binding moiety comprises (a) a heavy chain variable region (VH) comprising an amino acid sequence that is at least about 95%, 96%, 97%, 98%, 99% or 100% identical to the amino acid sequence of SEQ ID NO:80, and (b) a light chain variable region (VL) comprising an amino acid sequence that is at least about 95%, 96%, 97%, 98%, 99% or 100% identical to the amino acid sequence of SEQ ID NO:81.

In some embodiments, the first antigen binding moiety comprises a VH sequence that is at least about 95%, 96%, 97%, 98%, 99% or 100% identical to the amino acid sequence of SEQ ID NO: 80, and a VL sequence that is at least about 95%, 96%, 97%, 98%, 99% or 100% identical to the amino acid sequence of SEQ ID NO: 81.

In some embodiments, the first antigen binding moiety comprises (a) a heavy chain variable region (VH) comprising the amino acid sequence of SEQ ID NO:80, and (b) a light chain variable region (VL) comprising the amino acid sequence of SEQ ID NO:81.

In some embodiments, the first antigen binding moiety comprises the VH sequence of SEQ ID NO: 80, and a VL sequence of SEQ ID NO: 81.

In one embodiment, the first antigen binding moiety comprises a human constant region. In one embodiment, the first antigen binding moiety is a Fab molecule comprising a human constant region, particularly a human CH1 and/or CL domain. In one embodiment, not more than one antigen binding moiety that binds to PD-1 is present in the bispecific antigen binding molecule (i.e. the bispecific antigen binding molecule provides monovalent binding to PD-1).

### Second Antigen Binding Moiety

The bispecific antigen binding molecule comprised in the immunoconjugate of the invention comprises at least one antigen binding moiety, particularly a Fab molecule, that binds to LAG3, particularly human LAG3 (second antigen).

In particular embodiments, the antigen binding moiety that binds to LAG3 is a conventional Fab molecule. In such embodiments, the antigen binding moiety that binds to PD-1 is preferably a crossover Fab molecule as described herein, i.e. a Fab molecule wherein the variable domains VH and VL or the constant domains CH1 and CL of the Fab heavy and light chains are exchanged/replaced by each other.

In alternative embodiments, the antigen binding moiety that binds to PD-1 is a conventional Fab molecule. In such embodiments, the antigen binding moiety that binds to LAG3 is a crossover Fab molecule as described herein, i.e. a Fab molecule wherein the variable domains VH and VL or the constant domains CH1 and CL of the Fab heavy and light chains are exchanged/replaced by each other.

In some embodiments, the second antigen binding moiety comprises a CDR-H1 comprising the amino acid sequence of SEQ ID NO:82, a CDR-H2 comprising the amino acid sequence of SEQ ID NO:83, a CDR-H3 comprising the amino acid sequence of SEQ ID NO:84, a CDR-L1 comprising the amino acid sequence of SEQ ID NO:85, a CDR-L2 comprising the amino acid sequence of SEQ ID NO:86, and a CDR-L3 comprising the amino acid sequence of SEQ ID NO:87.

In some embodiments, the second antigen binding moiety comprises (a) a heavy chain variable region (VH) comprising a CDR-H1 comprising the amino acid sequence of SEQ ID NO:82, a CDR-H2 comprising the amino acid sequence of SEQ ID NO:83, and a CDR-H3 comprising the amino acid sequence of SEQ ID NO:84, and (b) a light chain variable region (VL) comprising a CDR-L1 comprising the amino acid sequence of SEQ ID NO:85, a CDR-L2 comprising the amino acid sequence of SEQ ID NO:86, and a CDR-L3 comprising the amino acid sequence of SEQ ID NO:87.

In some embodiments, the second antigen binding moiety is (derived from) a humanized antibody. In one embodiment, the VH is a humanized VH and/or the VL is a humanized VL. In one embodiment, the second antigen binding moiety comprises CDRs as in any of the above embodiments, and further comprises an acceptor human framework, e.g. a human immunoglobulin framework or a human consensus framework. In some embodiments, the heavy and/or light chain variable region comprises human framework regions (FR).

In some embodiments, the second antigen binding moiety comprises a heavy chain variable region (VH) comprising an amino acid sequence that is at least about 95%, 96%, 97%, 98%, 99% or 100% identical to the amino acid sequence of SEQ ID NO:88. In some embodiments, the second antigen binding moiety comprises a light chain variable region (VL) comprising an amino acid sequence that is at least about 95%, 96%, 97%, 98%, 99% or 100% identical to the amino acid sequence of SEQ ID NO:89.

In some embodiments, the second antigen binding moiety comprises a VH sequence that is at least about 95%, 96%, 97%, 98%, 99% or 100% identical to the amino acid sequence of SEQ ID NO: 88, and a VL sequence that is at least about 95%, 96%, 97%, 98%, 99% or 100% identical to the amino acid sequence of SEQ ID NO: 89.

In a particular embodiment, the second antigen binding moiety comprises (a) a heavy chain variable region (VH) comprising the amino acid sequence of SEQ ID NO:88, and (b) a light chain variable region (VL) comprising the amino acid sequence of SEQ ID NO:89. In a further particular embodiment, the second antigen binding moiety comprises the VH sequence of SEQ ID NO: 88, and the VL sequence of SEQ ID NO: 89.

In one embodiment, the second antigen binding moiety comprises a human constant region. In one embodiment, the second antigen binding moiety is a Fab molecule comprising a human constant region, particularly a human CH1 and/or CL domain. Particularly, the light chain constant region may comprise amino acid mutations as described herein under “charge modifications” and/or may comprise deletion or substitutions of one or more (particularly two) N-terminal amino acids if in a crossover Fab molecule. Particularly, the heavy chain constant region (specifically CH1 domain) may comprise amino acid mutations as described herein under “charge modifications”.

In one embodiment, not more than one antigen binding moiety that binds to LAG3 is present in the bispecific antigen binding molecule (i.e. the bispecific antigen binding molecule provides monovalent binding to LAG3).

### Particular Aspects of the Invention—PD1-LAG3-IL2v Q126T

In a particular aspect, the invention provides an immunoconjugate comprising a mutant IL-2 polypeptide and a bispecific antigen binding molecule that binds to PD-1 and LAG3, wherein the mutant IL-2 polypeptide is a human IL-2 molecule comprising the amino acid substitutions F42A, Y45A, L72G and Q126T (numbering relative to the human IL-2 sequence SEQ ID NO: 90); and wherein the bispecific antigen binding molecule comprises (i) a first antigen binding moiety that binds to PD-1, comprising (a) a heavy chain variable region (VH) comprising the amino acid sequence that of SEQ ID NO:80, and (b) a light chain variable region (VL) comprising the amino acid sequence of SEQ ID NO:81; and (ii) a second antigen binding moiety that binds to LAG3, comprising (a) a heavy chain variable region (VH) comprising the amino acid sequence of SEQ ID NO:88, and (b) a light chain variable region (VL) comprising the amino acid sequence of SEQ ID NO:89.

In a particular aspect, the invention provides an immunoconjugate comprising a mutant IL-2 polypeptide and a bispecific antigen binding molecule that binds to PD-1 and LAG3, wherein the mutant IL-2 polypeptide is a human IL-2 molecule comprising the amino acid substitutions T3A, F42A, Y45A, L72G, C125A and Q126T (numbering relative to the human IL-2 sequence SEQ ID NO: 90); and wherein the bispecific antigen binding molecule comprises (i) a first antigen binding moiety that binds to PD-1, comprising (a) a heavy chain variable region (VH) comprising the amino acid sequence that of SEQ ID NO:80, and (b) a light chain variable region (VL) comprising the amino acid sequence of SEQ ID NO:81; and (ii) a second antigen binding moiety that binds to LAG3, comprising (a) a heavy chain variable region (VH) comprising the amino acid sequence of SEQ ID NO:88, and (b) a light chain variable region (VL) comprising the amino acid sequence of SEQ ID NO:89.

In a particular aspect, the invention provides an immunoconjugate comprising a mutant IL-2 polypeptide and a bispecific antigen binding molecule that binds to PD-1 and LAG3, wherein the mutant IL-2 polypeptide comprises the amino acid sequence of SEQ ID NO: 92; and wherein the bispecific antigen binding molecule comprises (i) a first antigen binding moiety that binds to PD-1, comprising (a) a heavy chain variable region (VH) comprising the amino acid sequence that of SEQ ID NO:80, and (b) a light chain variable region (VL) comprising the amino acid sequence of SEQ ID NO:81; and (ii) a second antigen binding moiety that binds to LAG3, comprising (a) a heavy chain variable region (VH) comprising the amino acid sequence of SEQ ID NO:88, and (b) a light chain variable region (VL) comprising the amino acid sequence of SEQ ID NO:89.

In some embodiments according to any one of the above aspects, the first antigen binding moiety is a Fab molecule wherein the variable domains VL and VH of the Fab light chain and the Fab heavy chain are replaced by each other, and the second antigen binding moiety is a (conventional) Fab molecule. In some such embodiments, in the constant domain CL of the second antigen binding moiety the amino acid at position 124 is substituted by lysine (K) (numbering according to Kabat) and the amino acid at position 123 is substituted by lysine (K) or arginine (R) (numbering according to Kabat) (most particularly by arginine (R)), and in the constant domain CH1 of the second antigen binding moiety the amino acid at position 147 is substituted by glutamic acid (E) (numbering according to Kabat EU index) and the amino acid at position 213 is substituted by glutamic acid (E) (numbering according to Kabat EU index).

In some embodiments according to any one of the above aspects, the bispecific antigen binding molecule further comprises an Fc domain composed of a first and a second subunit. In some such embodiments, the first antigen binding moiety is fused at the C-terminus of the Fab heavy chain to the N-terminus of one of the subunits of the Fc domain (particularly to the first subunit of the Fc domain), and the second antigen binding moiety is fused at C-terminus of the Fab heavy chain to the N-terminus of the other one of the subunits of the Fc domain (particularly to the second subunit of the Fc domain).

In a particular aspect, the invention provides an immunoconjugate comprising a mutant IL-2 polypeptide and a bispecific antigen binding molecule that binds to PD-1 and LAG3, wherein the mutant IL-2 polypeptide comprises the amino acid sequence of SEQ ID NO: 92; and wherein the bispecific antigen binding molecule comprises (i) a first antigen binding moiety that binds to PD-1, wherein the first antigen binding moiety is a Fab molecule wherein the variable domains VL and VH of the Fab light chain and the Fab heavy chain are replaced by each other, comprising (a) a heavy chain variable region (VH) comprising the amino acid sequence that of SEQ ID NO:80, and (b) a light chain variable region (VL) comprising the amino acid sequence of SEQ ID NO:81; (ii) a second antigen binding moiety that binds to LAG3, wherein the second antigen binding moiety is a (conventional) Fab molecule, comprising (a) a heavy chain variable region (VH) comprising the amino acid sequence of SEQ ID NO:88, and (b) a light chain variable region (VL) comprising the amino acid sequence of SEQ ID NO:89, wherein in the constant domain CL of the second antigen binding moiety the amino acid at position 124 is substituted by lysine (K) (numbering according to Kabat) and the amino acid at position 123 is substituted by lysine (K) or arginine (R) (numbering according to Kabat) (most particularly by arginine (R)), and in the constant domain CH1 of the second antigen binding moiety the amino acid at position 147 is substituted by glutamic acid (E) (numbering according to Kabat EU index) and the amino acid at position 213 is substituted by glutamic acid (E) (numbering according to Kabat EU index); and (iii) an Fc domain composed of a first and a second subunit, wherein the first antigen binding moiety is fused at the C-terminus of the Fab heavy chain to the N-terminus of one of the subunits of the Fc domain (particularly to the first subunit of the Fc domain), and the second antigen binding moiety is fused at C-terminus of the Fab heavy chain to the N-terminus of the other one of the subunits of the Fc domain (particularly to the second subunit of the Fc domain).

In some embodiments according to any of the above aspects of the invention, in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numberings according to Kabat EU index). In some such embodiments, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numberings according to Kabat EU index).

In some embodiments according to any of the above aspects of the invention, in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In some embodiments according to any of the above aspects of the invention, the Fc domain is a human IgG₁ Fc domain.

In some embodiments according to any of the above aspects of the invention, the mutant IL-2 polypeptide is fused at its amino-terminal amino acid to the carboxy-terminal amino acid of the first subunit of the Fc domain, through a linker peptide of SEQ ID NO: 93.

In particular specific embodiment, the immunoconjugate comprises a polypeptide comprising an amino acid sequence that is at least 95%, 96%, 97%, 98%, or 99% identical to the sequence of SEQ ID NO: 68, a polypeptide comprising an amino acid sequence that is at least 95%, 96%, 97%, 98%, or 99% identical to the sequence of SEQ ID NO: 69, a polypeptide comprising an amino acid sequence that is at least 95%, 96%, 97%, 98%, or 99% identical to the sequence of SEQ ID NO: 70, and a polypeptide comprising an amino acid sequence that is at least 95%, 96%, 97%, 98%, or 99% identical to the sequence of SEQ ID NO: 71. In a further particular specific embodiment, the bispecific antigen binding molecule comprises a polypeptide comprising the amino acid sequence of SEQ ID NO: 68, a polypeptide comprising the amino acid sequence of SEQ ID NO: 69, a polypeptide comprising the amino acid sequence of SEQ ID NO: 70 and a polypeptide comprising the amino acid sequence of SEQ ID NO: 71.

### Polynucleotides

The invention further provides isolated polynucleotides encoding an immunoconjugate as described herein or a fragment thereof. In some embodiments, said fragment is an antigen binding fragment.

The polynucleotides encoding immunoconjugates of the invention may be expressed as a single polynucleotide that encodes the entire immunoconjugate or as multiple (e.g., two or more) polynucleotides that are co-expressed. Polypeptides encoded by polynucleotides that are co-expressed may associate through, e.g., disulfide bonds or other means to form a functional immunoconjugate. For example, the light chain portion of an antibody may be encoded by a separate polynucleotide from the portion of the immunoconjugate comprising the heavy chain portion of the antibody and the mutant IL-2 polypeptide. When co-expressed, the heavy chain polypeptides will associate with the light chain polypeptides to form the immunoconjugate. In another example, the portion of the immunoconjugate comprising one of the two Fc domain subunits and the mutant IL-2 polypeptide could be encoded by a separate polynucleotide from the portion of the immunoconjugate comprising the the other of the two Fc domain subunits. When co-expressed, the Fc domain subunits will associate to form the Fc domain.

In some embodiments, the isolated polynucleotide encodes the entire immunoconjugate according to the invention as described herein. In other embodiments, the isolated polynucleotide encodes a polypeptide comprised in the immunoconjugate according to the invention as described herein.

In one embodiment, an isolated polynucleotide of the invention encodes the heavy chain of the antibody comprised in the immunoconjugate (e.g. an immunoglobulin heavy chain), and the mutant IL-2 polypeptide. In another embodiment, an isolated polynucleotide of the invention encodes the light chain of the antibody comprised in the immunoconjugate.

In certain embodiments the polynucleotide or nucleic acid is DNA. In other embodiments, a polynucleotide of the present invention is RNA, for example, in the form of messenger RNA (mRNA). RNA of the present invention may be single stranded or double stranded.

### Recombinant Methods

Mutant IL-2 polypeptides useful in the invention can be prepared by deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing. In this regard, the nucleotide sequence of native IL-2 has been described by Taniguchi et al. (Nature 302, 305-10 (1983)) and nucleic acid encoding human IL-2 is available from public depositories such as the American Type Culture Collection (Rockville MD). The sequence of native human IL-2 is shown in SEQ ID NO: 19. Substitution or insertion may involve natural as well as non-natural amino acid residues. Amino acid modification includes well known methods of chemical modification such as the addition of glycosylation sites or carbohydrate attachments, and the like.

Immunoconjugates of the invention may be obtained, for example, by solid-state peptide synthesis (e.g. Merrifield solid phase synthesis) or recombinant production. For recombinant production one or more polynucleotide encoding the immunoconjugate (fragment), e.g., as described above, is isolated and inserted into one or more vectors for further cloning and/or expression in a host cell. Such polynucleotide may be readily isolated and sequenced using conventional procedures. In one embodiment a vector, preferably an expression vector, comprising one or more of the polynucleotides of the invention is provided. Methods which are well known to those skilled in the art can be used to construct expression vectors containing the coding sequence of an immunoconjugate (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y (1989). The expression vector can be part of a plasmid, virus, or may be a nucleic acid fragment. The expression vector includes an expression cassette into which the polynucleotide encoding the immunoconjugate (fragment) (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the immunoconjugate of the invention, or variant or derivative thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit $-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. For example, human IL-2 is translated with a 20 amino acid signal sequence at the N-terminus of the polypeptide, which is subsequently cleaved off to produce the mature, 133 amino acid human IL-2. In certain embodiments, the native signal peptide, e.g. the IL-2 signal peptide or an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g. a histidine tag) or assist in labeling the immunoconjugate may be included within or at the ends of the immunoconjugate (fragment) encoding polynucleotide.

In a further embodiment, a host cell comprising one or more polynucleotides of the invention is provided. In certain embodiments a host cell comprising one or more vectors of the invention is provided. The polynucleotides and vectors may incorporate any of the features, singly or in combination, described herein in relation to polynucleotides and vectors, respectively. In one such embodiment a host cell comprises (e.g. has been transformed or transfected with) one or more vector comprising one or more polynucleotide that encodes the immunoconjugate of the invention. As used herein, the term “host cell” refers to any kind of cellular system which can be engineered to generate the immunoconjugates of the invention or fragments thereof. Host cells suitable for replicating and for supporting expression of immunoconjugates are well known in the art. Such cells may be transfected or transduced as appropriate with the particular expression vector and large quantities of vector containing cells can be grown for seeding large scale fermenters to obtain sufficient quantities of the immunoconjugate for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006). Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr CHO cells (Urlaub et al., Proc Natl Acad Sci USA 77, 4216 (1980)); and myeloma cell lines such as YO, NSO, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, NJ), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., Y0, NSO, Sp20 cell). Standard technologies are known in the art to express foreign genes in these systems. Cells expressing a mutant-IL-2 polypeptide fused to either the heavy or the light chain of an antibody may be engineered so as to also express the other of the antibody chains such that the expressed mutant IL-2 fusion product is an antibody that has both a heavy and a light chain.

In one embodiment, a method of producing an immunoconjugate according to the invention is provided, wherein the method comprises culturing a host cell comprising one or more polynucleotide encoding the immunoconjugate, as provided herein, under conditions suitable for expression of the immunoconjugate, and optionally recovering the immunoconjugate from the host cell (or host cell culture medium).

In the immunoconjugate of the invention, the mutant IL-2 polypeptide may be genetically fused to the antibody, or may be chemically conjugated to the antibody. Genetic fusion of the IL-2 polypeptide to the antibody can be designed such that the IL-2 sequence is fused directly to the polypeptide or indirectly through a linker sequence. The composition and length of the linker may be determined in accordance with methods well known in the art and may be tested for efficacy. Particular linker peptides are described herein. Additional sequences may also be included to incorporate a cleavage site to separate the individual components of the fusion if desired, for example an endopeptidase recognition sequence. In addition, an IL-2 fusion protein may also be synthesized chemically using methods of polypeptide synthesis as is well known in the art (e.g. Merrifield solid phase synthesis). Mutant IL-2 polypeptides may be chemically conjugated to other molecules, e.g. antibodies, using well known chemical conjugation methods. Bi-functional cross-linking reagents such as homofunctional and heterofunctional cross-linking reagents well known in the art can be used for this purpose. The type of cross-linking reagent to use depends on the nature of the molecule to be coupled to IL-2 and can readily be identified by those skilled in the art. Alternatively, or in addition, mutant IL-2 and/or the molecule to which it is intended to be conjugated may be chemically derivatized such that the two can be conjugated in a separate reaction as is also well known in the art.

The immunoconjugates of the invention comprise an antibody. Methods to produce antibodies are well known in the art (see e.g. Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g. as described in U.S. Pat. No. 4,186,567) or can be obtained, for example, by screening combinatorial libraries comprising variable heavy chains and variable light chains (see e.g. U.S. Pat. No. 5,969,108 to McCafferty). Immunoconjugates, antibodies, and methods for producing the same are also described in detail e.g. in PCT publication nos. WO 2011/020783, WO 2012/107417, and WO 2012/146628, each of which are incorporated herein by reference in their entirety.

Any animal species of antibody may be used in the immunoconjugates of the invention. Non-limiting antibodies useful in the present invention can be of murine, primate, or human origin. If the immunoconjugate is intended for human use, a chimeric form of antibody may be used wherein the constant regions of the antibody are from a human. A humanized or fully human form of the antibody can also be prepared in accordance with methods well known in the art (see e. g. U.S. Pat. No. 5,565,332 to Winter). Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling). Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

Human antibodies can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolation from human antibody libraries, as described herein.

Antibodies useful in the invention may be isolated by screening combinatorial libraries for antibodies with the desired activity or activities. Methods for screening combinatorial libraries are reviewed, e.g., in Lerner et al. in Nature Reviews 16:498-508 (2016). For example, a variety of methods are known in the art for generating phage display libraries and screening such libraries for antibodies possessing the desired binding characteristics. Such methods are reviewed, e.g., in Frenzel et al. in mAbs 8:1177-1194 (2016); Bazan et al. in Human Vaccines and Immunotherapeutics 8:1817-1828 (2012) and Zhao et al. in Critical Reviews in Biotechnology 36:276-289 (2016) as well as in Hoogenboom et al. in Methods in Molecular Biology 178:1-37 (O'Brien et al., ed., Human Press, Totowa, NJ, 2001) and in Marks and Bradbury in Methods in Molecular Biology 248:161-175 (Lo, ed., Human Press, Totowa, NJ, 2003).

In certain phage display methods, repertoires of VH and VL genes are separately cloned by polymerase chain reaction (PCR) and recombined randomly in phage libraries, which can then be screened for antigen-binding phage as described in Winter et al. in Annual Review of Immunology 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al. in EMBO Journal 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter in Journal of Molecular Biology 227: 381-388 (1992). Patent publications describing human antibody phage libraries include, for example: U.S. Pat. Nos. 5,750,373; 7,985,840; 7,785,903 and 8,679,490 as well as US Patent Publication Nos. 2005/0079574, 2007/0117126, 2007/0237764 and 2007/0292936. Further examples of methods known in the art for screening combinatorial libraries for antibodies with a desired activity or activities include ribosome and mRNA display, as well as methods for antibody display and selection on bacteria, mammalian cells, insect cells or yeast cells. Methods for yeast surface display are reviewed, e.g., in Scholler et al. in Methods in Molecular Biology 503:135-56 (2012) and in Cherf et al. in Methods in Molecular biology 1319:155-175 (2015) as well as in the Zhao et al. in Methods in Molecular Biology 889:73-84 (2012). Methods for ribosome display are described, e.g., in He et al. in Nucleic Acids Research 25:5132-5134 (1997) and in Hanes et al. in PNAS 94:4937-4942 (1997).

Further chemical modification of the immunoconjugate of the invention may be desirable. For example, problems of immunogenicity and short half-life may be improved by conjugation to substantially straight chain polymers such as polyethylene glycol (PEG) or polypropylene glycol (PPG) (see e.g. WO 87/00056).

Immunoconjugates prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the immunoconjugate binds. For example, an antibody which specifically binds the mutant IL-2 polypeptide may be used. For affinity chromatography purification of immunoconjugates of the invention, a matrix with protein A or protein G may be used. For example, sequential Protein A or G affinity chromatography and size exclusion chromatography can be used to isolate an immunoconjugate essentially as described in the Examples. The purity of the immunoconjugate can be determined by any of a variety of well known analytical methods including gel electrophoresis, high pressure liquid chromatography, and the like.

### Compositions, Formulations, and Routes of Administration

In a further aspect, the invention provides pharmaceutical compositions comprising an immunoconjugate as described herein, e.g., for use in any of the below therapeutic methods. In one embodiment, a pharmaceutical composition comprises any of the immunoconjugates provided herein and a pharmaceutically acceptable carrier. In another embodiment, a pharmaceutical composition comprises any of the immunoconjugates provided herein and at least one additional therapeutic agent, e.g., as described below.

Further provided is a method of producing an immunoconjugate of the invention in a form suitable for administration in vivo, the method comprising (a) obtaining an immunoconjugate according to the invention, and (b) formulating the immunoconjugate with at least one pharmaceutically acceptable carrier, whereby a preparation of immunoconjugate is formulated for administration in vivo.

Pharmaceutical compositions of the present invention comprise a therapeutically effective amount of immunoconjugate dissolved or dispersed in a pharmaceutically acceptable carrier. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains immunoconjugate and optionally an additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards or corresponding authorities in other countries. Preferred compositions are lyophilized formulations or aqueous solutions. As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

An immunoconjugate of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic.

Parenteral compositions include those designed for administration by injection, e.g. subcutaneous, intradermal, intralesional, intravenous, intraarterial intramuscular, intrathecal or intraperitoneal injection. For injection, the immunoconjugates of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the immunoconjugates may be in powder form for constitution with a suitable vehicle, e.g., sterile pyrogen-free water, before use. Sterile injectable solutions are prepared by incorporating the immunoconjugates of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable carriers include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

In addition to the compositions described previously, the immunoconjugates may also be formulated as a depot preparation. Such long acting formulations may be administered by implantation (for example subcutaneously or intramuscularly) or by intramuscular injection. Thus, for example, the immunoconjugates may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the immunoconjugates of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The immunoconjugates may be formulated into a composition in a free acid or base, neutral or salt form. Pharmaceutically acceptable salts are salts that substantially retain the biological activity of the free acid or base. These include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

### Therapeutic Methods and Compositions

Any of the immunoconjugates provided herein may be used in therapeutic methods. Immunoconjugates of the invention may be used as immunotherapeutic agents, for example in the treatment of cancers.

For use in therapeutic methods, immunoconjugates of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

Immunoconjugates of the invention may be particularly useful in treating disease states where stimulation of the immune system of the host is beneficial, in particular conditions where an enhanced cellular immune response is desirable. These may include disease states where the host immune response is insufficient or deficient. Disease states for which the immunoconjugates of the invention may be administered comprise, for example, a tumor or infection where a cellular immune response would be a critical mechanism for specific immunity. The immunoconjugates of the invention may be administered per se or in any suitable pharmaceutical composition.

In one aspect, immunoconjugates of the invention for use as a medicament are provided. In further aspects, immunoconjugates of the invention for use in treating a disease are provided. In certain embodiments, immunoconjugates of the invention for use in a method of treatment are provided. In one embodiment, the invention provides an immunoconjugate as described herein for use in the treatment of a disease in an individual in need thereof. In certain embodiments, the invention provides an immunoconjugate for use in a method of treating an individual having a disease comprising administering to the individual a therapeutically effective amount of the immunoconjugate. In certain embodiments the disease to be treated is a proliferative disorder. In a particular embodiment the disease is cancer. In certain embodiments the method further comprises administering to the individual a therapeutically effective amount of at least one additional therapeutic agent, e.g., an anti-cancer agent if the disease to be treated is cancer. In further embodiments, the invention provides an immunoconjugate for use in stimulating the immune system. In certain embodiments, the invention provides an immunoconjugate for use in a method of stimulating the immune system in an individual comprising administering to the individual an effective amount of the immunoconjugate to stimulate the immune system. An “individual” according to any of the above embodiments is a mammal, preferably a human. “Stimulation of the immune system” according to any of the above embodiments may include any one or more of a general increase in immune function, an increase in T cell function, an increase in B cell function, a restoration of lymphocyte function, an increase in the expression of IL-2 receptors, an increase in T cell responsiveness, an increase in natural killer cell activity or lymphokine-activated killer (LAK) cell activity, and the like.

In a further aspect, the invention provides for the use of an immunoconjugate of the invention in the manufacture or preparation of a medicament. In one embodiment, the medicament is for the treatment of a disease in an individual in need thereof. In one embodiment, the medicament is for use in a method of treating a disease comprising administering to an individual having the disease a therapeutically effective amount of the medicament. In certain embodiments the disease to be treated is a proliferative disorder. In a particular embodiment the disease is cancer. In one embodiment, the method further comprises administering to the individual a therapeutically effective amount of at least one additional therapeutic agent, e.g., an anti-cancer agent if the disease to be treated is cancer. In a further embodiment, the medicament is for stimulating the immune system. In a further embodiment, the medicament is for use in a method of stimulating the immune system in an individual comprising administering to the individual an effective amount of the medicament to stimulate the immune system. An “individual” according to any of the above embodiments may be a mammal, preferably a human. “Stimulation of the immune system” according to any of the above embodiments may include any one or more of a general increase in immune function, an increase in T cell function, an increase in B cell function, a restoration of lymphocyte function, an increase in the expression of IL-2 receptors, an increase in T cell responsiveness, an increase in natural killer cell activity or lymphokine-activated killer (LAK) cell activity, and the like.

In a further aspect, the invention provides a method for treating a disease in an individual. In one embodiment, the method comprises administering to an individual having such disease a therapeutically effective amount of an immunoconjugate of the invention. In one embodiment a composition is administered to said individual, comprising the immunoconjugate of the invention in a pharmaceutically acceptable form. In certain embodiments the disease to be treated is a proliferative disorder. In a particular embodiment the disease is cancer. In certain embodiments the method further comprises administering to the individual a therapeutically effective amount of at least one additional therapeutic agent, e.g., an anti-cancer agent if the disease to be treated is cancer. In a further aspect, the invention provides a method for stimulating the immune system in an individual, comprising administering to the individual an effective amount of an immunoconjugate to stimulate the immune system. An “individual” according to any of the above embodiments may be a mammal, preferably a human.

“Stimulation of the immune system” according to any of the above embodiments may include any one or more of a general increase in immune function, an increase in T cell function, an increase in B cell function, a restoration of lymphocyte function, an increase in the expression of IL-2 receptors, an increase in T cell responsiveness, an increase in natural killer cell activity or lymphokine-activated killer (LAK) cell activity, and the like.

In certain embodiments the disease to be treated is a proliferative disorder, particularly cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that may be treated using an immunoconjugate of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of kidney cancer, skin cancer, lung cancer, colorectal cancer, breast cancer, brain cancer, head and neck cancer, prostate cancer and bladder cancer. A skilled artisan readily recognizes that in many cases the immunoconjugates may not provide a cure but may only provide partial benefit. In some embodiments, a physiological change having some benefit is also considered therapeutically beneficial. Thus, in some embodiments, an amount of immunoconjugate that provides a physiological change is considered an “effective amount” or a “therapeutically effective amount”. The subject, patient, or individual in need of treatment is typically a mammal, more specifically a human.

In some embodiments, an effective amount of an immunoconjugate of the invention is administered to a cell. In other embodiments, a therapeutically effective amount of an immunoconjugates of the invention is administered to an individual for the treatment of disease.

For the prevention or treatment of disease, the appropriate dosage of an immunoconjugate of the invention (when used alone or in combination with one or more other additional therapeutic agents) will depend on the type of disease to be treated, the route of administration, the body weight of the patient, the type of molecule (e.g. comprising an Fc domain or not), the severity and course of the disease, whether the immunoconjugate is administered for preventive or therapeutic purposes, previous or concurrent therapeutic interventions, the patient's clinical history and response to the immunoconjugate, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The immunoconjugate is suitably administered to the patient at one time or over a series of treatments. Depending on the type and severity of the disease, about 1 μg/kg to 15 mg/kg (e.g. 0.1 mg/kg-10 mg/kg) of immunoconjugate can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the immunoconjugate would be in the range from about 0.005 mg/kg to about 10 mg/kg. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the immunoconjugate). An initial higher loading dose, followed by one or more lower doses may be administered. However, other dosage regimens may be useful. The progress of this therapy is easily monitored by conventional techniques and assays.

The immunoconjugates of the invention will generally be used in an amount effective to achieve the intended purpose. For use to treat or prevent a disease condition, the immunoconjugates of the invention, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the immunoconjugates which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by HPLC.

In cases of local administration or selective uptake, the effective local concentration of the immunoconjugates may not be related to plasma concentration. One having skill in the art will be able to optimize therapeutically effective local dosages without undue experimentation.

A therapeutically effective dose of the immunoconjugates described herein will generally provide therapeutic benefit without causing substantial toxicity. Toxicity and therapeutic efficacy of an immunoconjugate can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. Immunoconjugates that exhibit large therapeutic indices are preferred. In one embodiment, the immunoconjugate according to the present invention exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition. (See, e.g., Fingl et al., 1975, In: The Pharmacological Basis of Therapeutics, Ch. 1, p. 1, incorporated herein by reference in its entirety).

The attending physician for patients treated with immunoconjugates of the invention would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

The maximum therapeutic dose of an immunoconjugate comprising a mutant IL-2 polypeptide as described herein may be increased from those used for an immunoconjugate comprising wild-type IL-2.

### Other Agents and Treatments

The immunoconjugates according to the invention may be administered in combination with one or more other agents in therapy. For instance, an immunoconjugate of the invention may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers.

In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

Such other agents are suitably present in combination in amounts that are effective for the purpose intended. The effective amount of such other agents depends on the amount of immunoconjugate used, the type of disorder or treatment, and other factors discussed above. The immunoconjugates are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the immunoconjugate of the invention can occur prior to, simultaneously, and/or following, administration of the additional therapeutic agent and/or adjuvant. Immunoconjugates of the invention may also be used in combination with radiation therapy.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an immunoconjugate of the invention. The label or package insert indicates that the composition is used for treating the condition of choice. Moreover, the article of manufacture may comprise (a) a first container with a composition contained therein, wherein the composition comprises an immunoconjugate of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### BRIEF DESCRIPTION OF THE DRAWINGS

FIG. 1A and FIG. 1B. Murine surrogates of murine PD1-targeted IL2v constructs (targeted to murine PD1 using V-domains from rat). FIG. 1A shows P1AG9991, a bivalent murine PD1-targeted murine IgG1 DA PG with human IL2v fused to the C-terminus of the Fc DD-chain. FIG. 1B shows P1AG8304, a bivalent murine PD1-targeted murine IgG1 DA PG with human IL2v Q126T fused to the C-terminus of the Fc DD-chain.

FIG. 2A, FIG. 2B, and FIG. 2C. Human PD1-/LAG3-targeted IL2v constructs. FIG. 2A shows P1AF4801 a bispecific human PD1-/LAG3-targeted IgG1 PG LALA crossMab with human IL2v fused to the C-terminus of the Fc knob-chain. FIG. 2B shows P1AF7951, a bispecific human PD1-/LAG3-targeted IgG1 PG LALA crossMab with human IL2v Q126T fused to the C-terminus of the Fc knob-chain. FIG. 2C shows P1AA6888, a monospecific human PD1 IgG1 PG LALA used as control.

FIG. 3. Proliferation of NK92 cells upon treatment for 3 days with PD1-IL2v and several PD1-IL2v variants was determined by measuring ATP levels with CellTiter Glo.

FIG. 4A, FIG. FB, and FIG. 4C. Proliferation of CD8 T cells (FIG. 4A), NK cells (FIG. 4B) and CD4 T cells (FIG. 4C) within PBMCs upon treatment for 5 days with PD1-IL2v and several PD1-IL2v variants was determined by flow cytometry.

FIG. 5A, FIG. 5B, and FIG. 5C. Activation of CD8 T cells (FIG. 5A), NK cells (FIG. 5B) and CD4 T cells (FIG. 5C) within PBMCs upon treatment for 5 days with PD1-IL2v and several PD1-IL2v variants was determined by measuring CD25 upregulation by flow cytometry.

FIG. 6A, FIG. 6B, FIG. 6C, and FIG. 6D. Proliferation of CD8 T cells and NK cells within PBMCs upon treatment for 4 days with FAP-IL2v and several FAP-IL2v variants was determined by flow cytometry. FIG. 6A shows FAP-IL2v(G4S)₅, FAP-IL2v_D20T_Selectikine, FAP-IL2v_E215V, FAP-IL2v_E95A, FAP-IL2v_E95A and FAP-IL2v. FIG. 6B shows FAP-IL2v_L12A, FAP-IL2v_L12A_L19A, FAP-IL2v_T133K and FAP-IL2v. FIG. 6C shows FAP-IL2v_L12A_L80A, FAP-IL2v_L19V, FAP-IL2v_N88T, FAP-IL2v_N119K and FAP-IL2v. FIG. 6D shows FAP-IL2v_Q22A, FAP-IL2v_Q126T, FAP-IL2v_S87A, FAP-IL2v_S130A and FAP-IL2v.

FIG. 7A and FIG. 7B. Activation of CD8 T cells (FIG. 7A) and NK cells (FIG. 7B) within PBMCs upon treatment for 4 days with PD1-IL2v and several PD1-IL2v variants was determined by measuring CD25 upregulation by flow cytometry.

FIG. 8A and FIG. 8B. Activation of CD8 T cells (FIG. 8A) and NK (FIG. 8B) cells within PBMCs upon treatment for 4 days with PD1-IL2v and several PD1-IL2v variants was determined by measuring CD25 upregulation by flow cytometry.

FIG. 9A, FIG. 9B, and FIG. 9C. Proliferation of NK cells (FIG. 9A), CD8 T cells (FIG. 9B) and CD4 T cells (FIG. 9C) within PBMCs upon treatment for 5 days with FAP-IL2v and selected FAP-IL2v variants was determined by flow cytometry.

FIG. 10A, FIG. 10B, and FIG. 10C. Activation of NK cells (FIG. 10A), CD8 T cells (FIG. 10B) and CD4 T cells (FIG. 10C) within PBMCs upon treatment for 5 days with FAP-IL2v and selected FAP-IL2v variants was determined by measuring CD25 upregulation by flow cytometry.

FIG. 11A, FIG. 11B, FIG. 11C, and FIG. 11D. STAT5 phosphorylation in CD4 T cells (FIG. 11A), regulatory T cells (FIG. 11B), CD8 T cells (FIG. 11C) and NK cells (FIG. 11D) upon treatment of PBMCs with FAP-IL2v and selected FAP-IL2v variants was determined by flow cytometry.

FIG. 12A, FIG. 12B, and FIG. 12C. Proliferation of CD8 T cells (FIG. 12A), NK cells (FIG. 12B) and CD4 T cells (FIG. 12C) within PBMCs upon treatment for 5 days with FAP-IL2v, FAP-IL2v Q126T and PD1-IL2v Q126T was determined by flow cytometry.

FIG. 13A, FIG. 13B, and FIG. 13C. Activation of CD8 T cells (FIG. 13A), NK cells (FIG. 13B) and CD4 T cells (FIG. 13C) within PBMCs upon treatment for 5 days with FAP-IL2v, FAP-IL2v Q126T and PD1-IL2v Q126T was determined by measuring CD25 upregulation by flow cytometry.

FIG. 14A, FIG. 14B, FIG. 14C, FIG. 14D, FIG. 14E, FIG. 14F, and FIG. 14G. IL-2 signaling (STAT5-P) in PD1-blocked and PD-1 expressing CD4+ T cells cultured together. IL-2 signaling (STAT5-P) depicted as frequency of STAT5-P in human PD1+ (solid line) and PD-1 pre-blocked (dotted line) CD4 T cells upon 12 min exposure to PD1-IL2v mutants. Mean±SEM of 4 donors is shown.

FIG. 14A shows PD1-IL2v, PD1-IL2v and PD1-pre-blocked, PD1-IL2v_Q126T, PD1-IL2v_Q126T and PD1-pre-blocked, PD1-IL2v_K8S, PD1-IL2v_K8S and PD1-pre-blocked.

FIG. 14B shows PD1-IL2v, PD1-IL2v and PD1-pre-blocked, PD1-IL2v_Q126T, PD1-IL2v_Q126T and PD1-pre-blocked, PD1-IL2v_L12A, PD1-IL2v_L12A and PD1-pre-blocked.

FIG. 14C shows PD1-IL2v, PD1-IL2v and PD1-pre-blocked, PD1-IL2v_Q126T, PD1-IL2v_Q126T and PD1-pre-blocked, PD1-IL2v_N88D, PD1-IL2v_N88D and PD1-pre-blocked.

FIG. 14D shows PD1-IL2v, PD1-IL2v and PD1-pre-blocked, PD1-IL2v_Q126T, PD1-IL2v_Q126T and PD1-pre-blocked, PD1-IL2v_L19V, PD1-IL2v_L19V and PD1-pre-blocked.

FIG. 14E shows PD1-IL2v, PD1-IL2v and PD1-pre-blocked, PD1-IL2v_Q126T, PD1-IL2v_Q126T and PD1-pre-blocked, PD1-IL2v_H79S, PD1-IL2v_H79S and PD1-pre-blocked.

FIG. 14F shows PD1-IL2v, PD1-IL2v and PD1-pre-blocked, PD1-IL2v_Q126T, PD1-IL2v_Q126T and PD1-pre-blocked, PD1-IL2v_D109A, PD1-IL2v_D109A and PD1-pre-blocked.

FIG. 14G shows PD1-IL2v, PD1-IL2v and PD1-pre-blocked, PD1-IL2v_Q126T, PD1-IL2v_Q126T and PD1-pre-blocked, PD1-IL2v_L80A, PD1-IL2v_L80A and PD1-pre-blocked.

FIG. 15A and FIG. 15B. IL-2 signaling (STAT5-P) PD-1 expressing CD4+ T cells (FIG. 15A) show the frequency and mean florescent intensity (MFI) of the potency (FIG. 15B) of selected PD1−, FAP− and NKG2D-IL2 variants on PD-1+ CD4 T cells. The potency measurement in the PD1+ CD4 T cells reflects the PD1-mediated delivery of IL-2v versus the PD1-independent delivery of the FAP-IL-2v constructs. Mean±SEM of 4 donors is shown.

FIG. 16A and FIG. 16B. Frequency of CMV specific CD4 T cells upon restimulation with the CMV protein pp65 and in the presence of the indicated treatment in combination with pp65 (FIG. 16A). Fold increase in frequency of CMV specific CD4 T cells, by normalizing the respective response to pp65 alone shows, the specific compound effect in expanding antigen-specific T cell responses (FIG. 16B). Mean±SEM of 5 donors is shown.

FIG. 17. Percentage of Treg-mediated suppression of granzyme B production by T conv in a 5 days coculture in presence or absence of the indicated immunoconjugates. Median of 6 donors. P is calculated using one-way ANOVA (*p<0.05, **p<0.01, ***p<0.001, ****p<0.0001).

FIG. 18. IL-2 signaling (STAT5-P) PD-1 expressing CD4+ T cells. Frequency of the potency of selected PD1−, LAG-3, FAP-IL2 variants on PD-1+, LAG-3+ and PD-1−, LAG-3− CD4 T cells. The potency measurement in the PD1+, LAG-3+ CD4 T cells reflects the PD1-(LAG-3) mediated delivery of IL-2v versus the PD-1/LAG-3-independent delivery of the FAP-IL-2v constructs. Mean±SEM of 3 donors is shown.

FIG. 19. Percentage of Treg-mediated suppression of granzyme B production by T conv in a 5 days coculture in presence or absence of the indicated immuneconjugates. Median of 10 donors. P is calculated using one-way ANOVA (*p<0.05, **p<0.01, ***p<0.001, ****p<0.0001.

FIG. 20. Percentage of internalized molecules at 0.6 nM by activated CD4 T cells after 3 hours of incubation at 37° C. Median of 4 donors. P is calculated using one-way ANOVA (*p<0.05, **p<0.01, ***p<0.001, ****p<0.0001).

FIG. 21. Presents the results of an efficacy experiment with murine surrogates for PD1-IL2vQ126T variant and PD-IL2v Mabs as single agents. The Panc02-Fluc pancreatic carcinoma cell line was injected subcutaneously in Black 6-huIL2RBG transgenics mice to study tumor growth inhibition (TGI) in a subcutaneous model. Tumor size was measured using a caliper. Therapy started when tumors reached 100 mm3. The amount of antibodies injected per mouse was 2 mg/kg for muPD1-IL2vQ126T variant and 0.5 mg/kg for muPD1-IL2v qw. The treatment lasted 3 weeks. The PD1-IL2vQ126T variant mediated significant superior efficacy in terms of tumor growth inhibition compared to vehicle and PD1-IL2v groups. The PD1-IL2vQ126T molecule was well tolerated and no clinical signs or body weight loss was observed.

| | Amino acid sequence | SEQ ID NO |
|---|---|---|
| FAP-targeted IL-2(Knob/hole) | EIVLTQSPGTLSLSPGERATLSCRASQSVTSSYLAW | 1 |
| (P1AA5355) - LC | YQQKPGQAPRLLINVGSRRATGIPDRFSGSGSGTDF | |
| | TLTISRLEPEDFAVYYCQQGIMLPPTFGQGTKVEIK | |
| | RTVAAPSVFIFPPSDEQLKSGTASVVCLLNNFYPRE | |
| | AKVQWKVDNALQSGNSQESVTEQDSKDSTYSLSSTL | |
| | TLSKADYEKHKVYACEVTHQGLSSPVTKSFNRGEC | |
| FAP-targeted IL-2(Knob/hole) | EVQLLESGGGLVQPGGSLRLSCAASGFTFSSYAMSW | 2 |
| (P1AA5355) - HC hole | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVCTLPPSRDELTK | |
| | NQVSLSCAVKGFYPSDIAVEWESNGQPENNYKTTPP | |
| | VLDSDGSFFLVSKLTVDKSRWQQGNVFSCSVMHEAL | |
| | HNHYTQKSLSLSPGK | |
| FAP-targeted IL-2(Knob/hole) | EVQLLESGGGLVQPGGSLRLSCAASGFTFSSYAMSW | 3 |
| (P1AA5355) - HC knob | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVYTLPPCRDELTK | |
| | NQVSLWCLVKGFYPSDIAVEWESNGQPENNYKTTPP | |
| | VLDSDGSFFLYSKLTVDKSRWQQGNVFSCSVMHEAL | |
| | HNHYTQKSLSLSPGGGGGSGGGGSGGGGSAPASSST | |
| | KKTQLQLEHLLLDLQMILNGINNYKNPKLTRMLTAK | |
| | FAMPKKATELKHLQCLEEELKPLEEVLNGAQSKNFH | |
| | LRPRDLISNINVIVLELKGSETTFMCEYADETATIV | |
| | EFLNRWITFAQSIISTLT | |
| huPD1-IL2v (w/ huPD1-Lead | DIVMTQSPDSLAVSLGERATINCKASESVDTSDNSF | 4 |
| 0376) (P1AD5438) - LC | IHWYQQKPGQSPKLLIYRSSTLESGVPDRFSGSGSG | |
| | TDFTLTISSLQAEDVAVYYCQQNYDVPWTFGQGTKV | |
| | EIKRTVAAPSVFIFPPSDEQLKSGTASVVCLLNNFY | |
| | PREAKVQWKVDNALQSGNSQESVTEQDSKDSTYSLS | |
| | STLTLSKADYEKHKVYACEVTHQGLSSPVTKSFNRG | |
| | EC | |
| huPD1-IL2v (w/ huPD1-Lead | EVQLLESGGGLVQPGGSLRLSCAASGFSFSSYTMSW | 5 |
| 0376) (P1AD5438) - HC hole | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVCTLPPSRDE | |
| | LTKNQVSLSCAVKGFYPSDIAVEWESNGQPENNYKT | |
| | TPPVLDSDGSFFLVSKLTVDKSRWQQGNVFSCSVMH | |
| | EALHNRFTQKSLSLSPGK | |
| huPD1-IL2v (w/ huPD1-Lead | EVQLLESGGGLVQPGGSLRLSCAASGFSFSSYTMSW | 6 |
| 0376) (P1AD5438) - HC knob | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVYTLPPCRDE | |
| | LTKNQVSLWCLVKGFYPSDIAVEWESNGQPENNYKT | |
| | TPPVLDSDGSFFLYSKLTVDKSRWQQGNVFSCSVMH | |
| | EALHNHYTQKSLSLSPGGGGGSGGGGSGGGGSAPAS | |
| | SSTKKTQLQLEHLLLDLQMILNGINNYKNPKLTRML | |
| | TAKFAMPKKATELKHLQCLEEELKPLEEVLNGAQSK | |
| | NFHLRPRDLISNINVIVLELKGSETTFMCEYADETA | |
| | TIVEFLNRWITFAQSIISTLT | |
| FAP 4B9 IgG-huIL2v (IL2qm) | EIVLTQSPGTLSLSPGERATLSCRASQSVTSSYLAW | 1 |
| (P1AE4798) - LC | YQQKPGQAPRLLINVGSRRATGIPDRFSGSGSGTDF | |
| | TLTISRLEPEDFAVYYCQQGIMLPPTFGQGTKVEIK | |
| | RTVAAPSVFIFPPSDEQLKSGTASVVCLLNNFYPRE | |
| | AKVQWKVDNALQSGNSQESVTEQDSKDSTYSLSSTL | |
| | TLSKADYEKHKVYACEVTHQGLSSPVTKSFNRGEC | |
| FAP 4B9 IgG-huIL2v (IL2qm) | EVQLLESGGGLVQPGGSLRLSCAASGFTFSSYAMSW | 2 |
| (P1AE4798) - HC hole | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVCTLPPSRDELTK | |
| | NQVSLSCAVKGFYPSDIAVEWESNGQPENNYKTTPP | |
| | VLDSDGSFFLVSKLTVDKSRWQQGNVFSCSVMHEAL | |
| | HNHYTQKSLSLSPGK | |
| FAP 4B9 IgG-huIL2v (IL2qm) | EVQLLESGGGLVQPGGSLRLSCAASGFTFSSYAMSW | 7 |
| (P1AE4798) - HC knob | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVYTLPPCRDELTK | |
| | NQVSLWCLVKGFYPSDIAVEWESNGQPENNYKTTPP | |
| | VLDSDGSFFLYSKLTVDKSRWQQGNVFSCSVMHEAL | |
| | HNHYTQKSLSLSPGGGGGSGGGGSGGGGSGGGGSGG | |
| | GGSAPASSSTKKTQLQLEHLLLDLQMILNGINNYKN | |
| | PKLTRMLTAKFAMPKKATELKHLQCLEEELKPLEEV | |
| | LNGAQSKNFHLRPRDLISNINVIVLELKGSETTFMC | |
| | EYADETATIVEFLNRWITFAQSIISTLT | |
| FAP 4B9 IgG- | EIVLTQSPGTLSLSPGERATLSCRASQSVTSSYLAW | 1 |
| huIL2v_D20T_Selectikine | YQQKPGQAPRLLINVGSRRATGIPDRFSGSGSGTDF | |
| (P1AE4799) - LC | TLTISRLEPEDFAVYYCQQGIMLPPTFGQGTKVEIK | |
| | RTVAAPSVFIFPPSDEQLKSGTASVVCLLNNFYPRE | |
| | AKVQWKVDNALQSGNSQESVTEQDSKDSTYSLSSTL | |
| | TLSKADYEKHKVYACEVTHQGLSSPVTKSFNRGEC | |
| FAP 4B9 IgG- | EVQLLESGGGLVQPGGSLRLSCAASGFTFSSYAMSW | 2 |
| huIL2v_D20T_Selectikine | VRQAPGKGLEWVSAIIGSGASTYYADSVKGRFTISR | |
| (P1AE4799) - HC hole | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVCTLPPSRDELTK | |
| | NQVSLSCAVKGFYPSDIAVEWESNGQPENNYKTTPP | |
| | VLDSDGSFFLVSKLTVDKSRWQQGNVFSCSVMHEAL | |
| | HNHYTQKSLSLSPGK | |
| FAP 4B9 IgG- | EVQLLESGGGLVQPGGSLRLSCAASGFTFSSYAMSW | 8 |
| huIL2v_D20T_Selectikine | VRQAPGKGLEWVSAIIGSGASTYYADSVKGRFTISR | |
| (P1AE4799) - HC knob | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVYTLPPCRDELTK | |
| | NQVSLWCLVKGFYPSDIAVEWESNGQPENNYKTTPP | |
| | VLDSDGSFFLYSKLTVDKSRWQQGNVFSCSVMHEAL | |
| | HNHYTQKSLSLSPGGGGGSGGGGSGGGGSGGGGSGG | |
| | GGSAPASSSTKKTQLQLEHLLLTLQMILNGINNYKN | |
| | PKLTRMLTAKFAMPKKATELKHLQCLEEELKPLEEV | |
| | LNGAQSKNFHLRPRDLISNINVIVLELKGSETTFMC | |
| | EYADETATIVEFLNRWITFAQSIISTLT | |
| FAP 4B9 IgG-huIL2_IL2Ra | EIVLTQSPGTLSLSPGERATLSCRASQSVTSSYLAW | 1 |
| (P1AE4802) - LC | YQQKPGQAPRLLINVGSRRATGIPDRFSGSGSGTDF | |
| | TLTISRLEPEDFAVYYCQQGIMLPPTFGQGTKVEIK | |
| | RTVAAPSVFIFPPSDEQLKSGTASVVCLLNNFYPRE | |
| | AKVQWKVDNALQSGNSQESVTEQDSKDSTYSLSSTL | |
| | TLSKADYEKHKVYACEVTHQGLSSPVTKSFNRGEC | |
| FAP 4B9 IgG-huIL2_IL2Ra | EVQLLESGGGLVQPGGSLRLSCAASGFTFSSYAMSW | 2 |
| (P1AE4802) - HC hole | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVCTLPPSRDELTK | |
| | NQVSLSCAVKGFYPSDIAVEWESNGQPENNYKTTPP | |
| | VLDSDGSFFLVSKLTVDKSRWQQGNVFSCSVMHEAL | |
| | HNHYTQKSLSLSPGK | |
| FAP 4B9 IgG-huIL2_IL2Ra | EVQLLESGGGLVQPGGSLRLSCAASGFTFSSYAMSW | 9 |
| (P1AE4802) - HC knob | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVYTLPPCRDELTK | |
| | NQVSLWCLVKGFYPSDIAVEWESNGQPENNYKTTPP | |
| | VLDSDGSFFLYSKLTVDKSRWQQGNVFSCSVMHEAL | |
| | HNHYTQKSLSLSPGGGGGSGGGGSGGGGSGGGGSGG | |
| | GGSAPASSSTKKTQLQLEHLLLDLQMILNGINNYKN | |
| | PKLTRMLTFKFYMPKKATELKHLQCLEEELKPLEEV | |
| | LNLAQSKNFHLRPRDLISNINVIVLELKGSETTFMC | |
| | EYADETATIVEFLNRWITFSQSIISTLTGGGGSGGG | |
| | GSGGGGSGGGGSELCDDDPPEIPHATFKAMAYKEGT | |
| | MLNCECKRGFRRIKSGSLYMLCTGNSSHSSWDNQCQ | |
| | CTSSATRNTTKQVTPQPEEQKERKTTEMQSPMQPVD | |
| | QASLPGHCREPPPWENEATERIYHFVVGQMVYYQCV | |
| | QGYRALHRGPAESVCKMTHGKTRWTQPQLICTGE | |
| FAP 4B9 IgG-IL2Ra_IL2 | EIVLTQSPGTLSLSPGERATLSCRASQSVTSSYLAW | 1 |
| (P1AE4803) - LC | YQQKPGQAPRLLINVGSRRATGIPDRFSGSGSGTDF | |
| | TLTISRLEPEDFAVYYCQQGIMLPPTFGQGTKVEIK | |
| | RTVAAPSVFIFPPSDEQLKSGTASVVCLLNNFYPRE | |
| | AKVQWKVDNALQSGNSQESVTEQDSKDSTYSLSSTL | |
| | TLSKADYEKHKVYACEVTHQGLSSPVTKSFNRGEC | |
| FAP 4B9 IgG-IL2Ra_IL2 | EVQLLESGGGLVQPGGSLRLSCAASGFTFSSYAMSW | 2 |
| (P1AE4803) - HC hole | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVCTLPPSRDELTK | |
| | NQVSLSCAVKGFYPSDIAVEWESNGQPENNYKTTPP | |
| | VLDSDGSFFLVSKLTVDKSRWQQGNVFSCSVMHEAL | |
| | HNHYTQKSLSLSPGK | |
| FAP 4B9 IgG-IL2Ra_IL2 | EVQLLESGGGLVQPGGSLRLSCAASGFTFSSYAMSW | 10 |
| (P1AE4803) HC knob | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVYTLPPCRDELTK | |
| | NQVSLWCLVKGFYPSDIAVEWESNGQPENNYKTTPP | |
| | VLDSDGSFFLYSKLTVDKSRWQQGNVFSCSVMHEAL | |
| | HNHYTQKSLSLSPGGGGGSGGGGSGGGGSGGGGSGG | |
| | GGSELCDDDPPEIPHATFKAMAYKEGTMLNCECKRG | |
| | FRRIKSGSLYMLCTGNSSHSSWDNQCQCTSSATRNT | |
| | TKQVTPQPEEQKERKTTEMQSPMQPVDQASLPGHCR | |
| | EPPPWENEATERIYHFVVGQMVYYQCVQGYRALHRG | |
| | PAESVCKMTHGKTRWTQPQLICTGEMETSQFPGEEK | |
| | PQASPEGRPESETSSLVTTTDFQIQTEMAATMETSI | |
| | FTTEGGGGSGGGGSGGGGSGGGGSAPASSSTKKTQL | |
| | QLEHLLLDLQMILNGINNYKNPKLTRMLTFKFYMPK | |
| | KATELKHLQCLEEELKPLEEVLNLAQSKNFHLRPRD | |
| | LISNINVIVLELKGSETTFMCEYADETATIVEFLNR | |
| | WITFSQSIISTLT | |
| FAP 4B9 IgG-huIL2v_Superkine | EIVLTQSPGTLSLSPGERATLSCRASQSVTSSYLAW | 1 |
| (P1AE4815) - LC | YQQKPGQAPRLLINVGSRRATGIPDRFSGSGSGTDF | |
| | TLTISRLEPEDFAVYYCQQGIMLPPTFGQGTKVEIK | |
| | RTVAAPSVFIFPPSDEQLKSGTASVVCLLNNFYPRE | |
| | AKVQWKVDNALQSGNSQESVTEQDSKDSTYSLSSTL | |
| | TLSKADYEKHKVYACEVTHQGLSSPVTKSFNRGEC | |
| FAP 4B9 IgG-huIL2v_Superkine | EVQLLESGGGLVQPGGSLRLSCAASGFTFSSYAMSW | 2 |
| (P1AE4815) - HC hole | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVCTLPPSRDELTK | |
| | NQVSLSCAVKGFYPSDIAVEWESNGQPENNYKTTPP | |
| | VLDSDGSFFLVSKLTVDKSRWQQGNVFSCSVMHEAL | |
| | HNHYTQKSLSLSPGK | |
| FAP 4B9 IgG-huIL2v_Superkine | EVQLLESGGGLVQPGGSLRLSCAASGFTFSSYAMSW | 11 |
| (P1AE4815) - HC knob | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVYTLPPCRDELTK | |
| | NQVSLWCLVKGFYPSDIAVEWESNGQPENNYKTTPP | |
| | VLDSDGSFFLYSKLTVDKSRWQQGNVFSCSVMHEAL | |
| | HNHYTQKSLSLSPGGGGGSGGGGSGGGGSGGGGSGG | |
| | GGSAPASSSTKKTQLQLEHLLLDLQMILNGINNYKN | |
| | PKLTRMLTAKFAMPKKATELKHLQCLEEELKPLEEV | |
| | LNGAHSKNFHFDPRDVVSNINVFVLELKGSETTFMC | |
| | EYADETATIVEFLNRWITFAQSIISTLT | |
| PD1 0379 IgG-IL2v (P1AD4370) - | DIQMTQSPSSLSASVGDRVTITCRASESVDTSDNSF | 12 |
| LC | IHWYQQKPGKSPKLLIYRSSTLESGVPSRFSGSGSG | |
| | TDFTLTISSLOPEDFATYYCQQNYDVPWTFGQGTKV | |
| | EIKRTVAAPSVFIFPPSDEQLKSGTASVVCLLNNFY | |
| | PREAKVQWKVDNALQSGNSQESVTEQDSKDSTYSLS | |
| | STLTLSKADYEKHKVYACEVTHQGLSSPVTKSFNRG | |
| | EC | |
| PD1 0379 IgG-IL2v (P1AD4370) - | EVQLLESGGGLVQPGGSLRLSCAASGFSFSSYTMSW | 13 |
| HC hole | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVCTLPPSRDE | |
| | LTKNQVSLSCAVKGFYPSDIAVEWESNGQPENNYKT | |
| | TPPVLDSDGSFFLVSKLTVDKSRWQQGNVFSCSVMH | |
| | EALHNRFTQKSLSLSPGK | |
| PD1 0379 IgG-IL2v (P1AD4370) - | EVQLLESGGGLVQPGGSLRLSCAASGFSFSSYTMSW | 14 |
| HC knob | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVYTLPPCRDE | |
| | LTKNQVSLWCLVKGFYPSDIAVEWESNGQPENNYKT | |
| | TPPVLDSDGSFFLYSKLTVDKSRWQQGNVFSCSVMH | |
| | EALHNHYTQKSLSLSPGGGGGSGGGGSGGGGSAPAS | |
| | SSTKKTQLQLEHLLLDLQMILNGINNYKNPKLTRML | |
| | TAKFAMPKKATELKHLQCLEEELKPLEEVLNGAQSK | |
| | NFHLRPRDLISNINVIVLELKGSETTFMCEYADETA | |
| | TIVEFLNRWITFAQSIISTLT | |
| NKG2D-IL2v alias NKG2D- | DIQMTQSPSSLSASVGDRVTITCRASQGISNDLAWY | 15 |
| IL2QM-F-11-01 (P1AE2767) - LC | QQKPGKAPKLLIYAASSLQSGVPSRFSGSGSGTDFT | |
| | LTISSLQPEDFATYYCQQTYSTPLTFGGGTKVEIKR | |
| | TVAAPSVFIFPPSDEQLKSGTASVVCLLNNFYPREA | |
| | KVQWKVDNALQSGNSQESVTEQDSKDSTYSLSSTLT | |
| | LSKADYEKHKVYACEVTHQGLSSPVTKSFNRGEC | |
| NKG2D-IL2v alias NKG2D- | EVQLLESGGGLVQPGGSLRLSCAASGFTFSSYAMSW | 16 |
| IL2QM-F-11-01 (P1AE2767) - HC | VRQAPGKGLEWVSAIGIGGGGTYYADSVKGRFTISR | |
| hole | DNSKNTLYLQMNSLRAEDTAVYHCAKGASFDFINFF | |
| | PYWGQGTLVTVSSASTKGPSVFPLAPSSKSTSGGTA | |
| | ALGCLVKDYFPEPVTVSWNSGALTSGVHTFPAVLQS | |
| | SGLYSLSSVVTVPSSSLGTQTYICNVNHKPSNTKVD | |
| | KKVEPKSCDKTHTCPPCPAPEAAGGPSVFLFPPKPK | |
| | DTLMISRTPEVTCVVVDVSHEDPEVKFNWYVDGVEV | |
| | HNAKTKPREEQYNSTYRVVSVLTVLHQDWLNGKEYK | |
| | CKVSNKALGAPIEKTISKAKGQPREPQVCTLPPSRD | |
| | ELTKNQVSLSCAVKGFYPSDIAVEWESNGQPENNYK | |
| | TTPPVLDSDGSFFLVSKLTVDKSRWQQGNVFSCSVM | |
| | HEALHNHYTQKSLSLSPGK | |
| NKG2D-IL2v alias NKG2D- | EVQLLESGGGLVQPGGSLRLSCAASGFTFSSYAMSW | 17 |
| IL2QM-F-11-01 (P1AE2767) - HC | VRQAPGKGLEWVSAIGIGGGGTYYADSVKGRFTISR | |
| knob | DNSKNTLYLQMNSLRAEDTAVYHCAKGASFDFINFF | |
| | PYWGQGTLVTVSSASTKGPSVFPLAPSSKSTSGGTA | |
| | ALGCLVKDYFPEPVTVSWNSGALTSGVHTFPAVLQS | |
| | SGLYSLSSVVTVPSSSLGTQTYICNVNHKPSNTKVD | |
| | KKVEPKSCDKTHTCPPCPAPEAAGGPSVFLFPPKPK | |
| | DTLMISRTPEVTCVVVDVSHEDPEVKFNWYVDGVEV | |
| | HNAKTKPREEQYNSTYRVVSVLTVLHQDWLNGKEYK | |
| | CKVSNKALGAPIEKTISKAKGQPREPQVYTLPPCRD | |
| | ELTKNQVSLWCLVKGFYPSDIAVEWESNGQPENNYK | |
| | TTPPVLDSDGSFFLYSKLTVDKSRWQQGNVFSCSVM | |
| | HEALHNHYTQKSLSLSPGGGGGSGGGGSGGGGSAPA | |
| | SSSTKKTQLQLEHLLLDLQMILNGINNYKNPKLTRM | |
| | LTAKFAMPKKATELKHLQCLEEELKPLEEVLNGAQS | |
| | KNFHLRPRDLISNINVIVLELKGSETTFMCEYADET | |
| | ATIVEFLNRWITFAQSIISTLT | |
| PD1TIM3-0591-0002 (P1AA6761, | DIVMTQSPDSLAVSLGERATINCKASESVDTSDNSF | 18 |
| P1AD5401) - LC | IHWYQQKPGQSPKLLIYRSSTLESGVPDRFSGSGSG | |
| | TDFTLTISSLQAEDVAVYYCQQNYDVPWTFGQGTKV | |
| | EIKRTVAAPSVFIFPPSDEQLKSGTASVVCLLNNFY | |
| | PREAKVQWKVDNALQSGNSQESVTEQDSKDSTYSLS | |
| | STLTLSKADYEKHKVYACEVTHQGLSSPVTKSFNRG | |
| | EC | |
| PD1TIM3-0591-0002 (P1AA6761, | EVQLLESGGGLVQPGGSLRLSCAASGFSFSSYTMSW | 19 |
| P1AD5401) - HC hole | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVCTLPPSRDE | |
| | LTKNQVSLSCAVKGFYPSDIAVEWESNGQPENNYKT | |
| | TPPVLDSDGSFFLVSKLTVDKSRWQQGNVFSCSVMH | |
| | EALHNRFTQKSLSLSPGK | |
| PD1TIM3-0591-0002 (P1AA6761, | EVQLLESGGGLVQPGGSLRLSCAASGFSFSSYTMSW | 20 |
| P1AD5401) - HC knob | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVYTLPPCRDE | |
| | LTKNQVSLWCLVKGFYPSDIAVEWESNGQPENNYKT | |
| | TPPVLDSDGSFFLYSKLTVDKSRWQQGNVFSCSVMH | |
| | EALHNHYTQKSLSLSPGGGGGSGGGGSGGGGSAPAS | |
| | SSTKKTQLQLEHLLLDLQMILNGINNYKNPKLTRML | |
| | TAKFAMPKKATELKHLQCLEEELKPLEEVLNGAQSK | |
| | NFHLRPRDLISDINVIVLELKGSETTFMCEYADETA | |
| | TIVEFLNRWITFAQSIISTLT | |
| PD1 376 IgG-huIL2v K8S | DIVMTQSPDSLAVSLGERATINCKASESVDTSDNSF | 21 |
| (P1AE0488) - LC | IHWYQQKPGQSPKLLIYRSSTLESGVPDRFSGSGSG | |
| | TDFTLTISSLQAEDVAVYYCQQNYDVPWTFGQGTKV | |
| | EIKRTVAAPSVFIFPPSDEQLKSGTASVVCLLNNFY | |
| | PREAKVQWKVDNALQSGNSQESVTEQDSKDSTYSLS | |
| | STLTLSKADYEKHKVYACEVTHQGLSSPVTKSFNRG | |
| | EC | |
| PD1 376 IgG-huIL2v K8S | EVQLLESGGGLVQPGGSLRLSCAASGFSFSSYTMSW | 22 |
| (P1AE0488) - HC hole | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVCTLPPSRDE | |
| | LTKNQVSLSCAVKGFYPSDIAVEWESNGQPENNYKT | |
| | TPPVLDSDGSFFLVSKLTVDKSRWQQGNVFSCSVMH | |
| | EALHNHYTQKSLSLSPGK | |
| PD1 376 IgG-huIL2v K8S | EVQLLESGGGLVQPGGSLRLSCAASGFSFSSYTMSW | 23 |
| (P1AE0488) - HC knob | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVYTLPPCRDE | |
| | LTKNQVSLWCLVKGFYPSDIAVEWESNGQPENNYKT | |
| | TPPVLDSDGSFFLYSKLTVDKSRWQQGNVFSCSVMH | |
| | EALHNHYTQKSLSLSPGGGGGSGGGGSGGGGSAPAS | |
| | SSTSKTQLQLEHLLLDLQMILNGINNYKNPKLTRML | |
| | TAKFAMPKKATELKHLQCLEEELKPLEEVLNGAQSK | |
| | NFHLRPRDLISNINVIVLELKGSETTFMCEYADETA | |
| | TIVEFLNRWITFAQSIISTLT | |
| PD1 376 IgG-huIL2v L12V | DIVMTQSPDSLAVSLGERATINCKASESVDTSDNSF | 21 |
| (P1AE0489) - LC | IHWYQQKPGQSPKLLIYRSSTLESGVPDRFSGSGSG | |
| | TDFTLTISSLQAEDVAVYYCQQNYDVPWTFGQGTKV | |
| | EIKRTVAAPSVFIFPPSDEQLKSGTASVVCLLNNFY | |
| | PREAKVQWKVDNALQSGNSQESVTEQDSKDSTYSLS | |
| | STLTLSKADYEKHKVYACEVTHQGLSSPVTKSFNRG | |
| | EC | |
| PD1 376 IgG-huIL2v L12V | EVQLLESGGGLVQPGGSLRLSCAASGFSFSSYTMSW | 22 |
| (P1AE0489) - HC hole | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVCTLPPSRDE | |
| | LTKNQVSLSCAVKGFYPSDIAVEWESNGQPENNYKT | |
| | TPPVLDSDGSFFLVSKLTVDKSRWQQGNVFSCSVMH | |
| | EALHNHYTQKSLSLSPGK | |
| PD1 376 IgG-huIL2v L12V | EVQLLESGGGLVQPGGSLRLSCAASGFSFSSYTMSW | 24 |
| (P1AE0489) - HC knob | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVYTLPPCRDE | |
| | LTKNQVSLWCLVKGFYPSDIAVEWESNGQPENNYKT | |
| | TPPVLDSDGSFFLYSKLTVDKSRWQQGNVFSCSVMH | |
| | EALHNHYTQKSLSLSPGGGGGSGGGGSGGGGSAPAS | |
| | SSTKKTQVQLEHLLLDLQMILNGINNYKNPKLTRML | |
| | TAKFAMPKKATELKHLQCLEEELKPLEEVLNGAQSK | |
| | NFHLRPRDLISNINVIVLELKGSETTFMCEYADETA | |
| | TIVEFLNRWITFAQSIISTLT | |
| PD1 376 IgG-huIL2v L12A | DIVMTQSPDSLAVSLGERATINCKASESVDTSDNSF | 21 |
| (P1AE0490) - LC | IHWYQQKPGQSPKLLIYRSSTLESGVPDRFSGSGSG | |
| | TDFTLTISSLQAEDVAVYYCQQNYDVPWTFGQGTKV | |
| | EIKRTVAAPSVFIFPPSDEQLKSGTASVVCLLNNFY | |
| | PREAKVQWKVDNALQSGNSQESVTEQDSKDSTYSLS | |
| | STLTLSKADYEKHKVYACEVTHQGLSSPVTKSFNRG | |
| | EC | |
| PD1 376 IgG-huIL2v L12A | EVQLLESGGGLVQPGGSLRLSCAASGFSFSSYTMSW | 22 |
| (P1AE0490) - HC hole | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVCTLPPSRDE | |
| | LTKNQVSLSCAVKGFYPSDIAVEWESNGQPENNYKT | |
| | TPPVLDSDGSFFLVSKLTVDKSRWQQGNVFSCSVMH | |
| | EALHNHYTQKSLSLSPGK | |
| PD1 376 IgG-huIL2v L12A | EVQLLESGGGLVQPGGSLRLSCAASGFSFSSYTMSW | 25 |
| (P1AE0490) - HC knob | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVYTLPPCRDE | |
| | LTKNQVSLWCLVKGFYPSDIAVEWESNGQPENNYKT | |
| | TPPVLDSDGSFFLYSKLTVDKSRWQQGNVFSCSVMH | |
| | EALHNHYTQKSLSLSPGGGGGSGGGGSGGGGSAPAS | |
| | SSTKKTQAQLEHLLLDLQMILNGINNYKNPKLTRML | |
| | TAKFAMPKKATELKHLQCLEEELKPLEEVLNGAQSK | |
| | NFHLRPRDLISNINVIVLELKGSETTFMCEYADETA | |
| | TIVEFLNRWITFAQSIISTLT | |
| PD1 376 IgG-huIL2v L19V | DIVMTQSPDSLAVSLGERATINCKASESVDTSDNSF | 21 |
| (P1AE0491) - LC | IHWYQQKPGQSPKLLIYRSSTLESGVPDRFSGSGSG | |
| | TDFTLTISSLQAEDVAVYYCQQNYDVPWTFGQGTKV | |
| | EIKRTVAAPSVFIFPPSDEQLKSGTASVVCLLNNFY | |
| | PREAKVQWKVDNALQSGNSQESVTEQDSKDSTYSLS | |
| | STLTLSKADYEKHKVYACEVTHQGLSSPVTKSFNRG | |
| | EC | |
| PD1 376 IgG-huIL2v L19V | EVQLLESGGGLVQPGGSLRLSCAASGFSFSSYTMSW | 22 |
| (P1AE0491) - HC hole | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVCTLPPSRDE | |
| | LTKNQVSLSCAVKGFYPSDIAVEWESNGQPENNYKT | |
| | TPPVLDSDGSFFLVSKLTVDKSRWQQGNVFSCSVMH | |
| | EALHNHYTQKSLSLSPGK | |
| PD1 376 IgG-huIL2v L19V | EVQLLESGGGLVQPGGSLRLSCAASGFSFSSYTMSW | 26 |
| (P1AE0491) - HC knob | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVYTLPPCRDE | |
| | LTKNQVSLWCLVKGFYPSDIAVEWESNGQPENNYKT | |
| | TPPVLDSDGSFFLYSKLTVDKSRWQQGNVFSCSVMH | |
| | EALHNHYTQKSLSLSPGGGGGSGGGGSGGGGSAPAS | |
| | SSTKKTQLQLEHLLVDLQMILNGINNYKNPKLTRML | |
| | TAKFAMPKKATELKHLQCLEEELKPLEEVLNGAQSK | |
| | NFHLRPRDLISNINVIVLELKGSETTFMCEYADETA | |
| | TIVEFLNRWITFAQSIISTLT | |
| PD1 376 IgG-huIL2v T51P | DIVMTQSPDSLAVSLGERATINCKASESVDTSDNSF | 21 |
| (P1AE0492) - LC | IHWYQQKPGQSPKLLIYRSSTLESGVPDRFSGSGSG | |
| | TDFTLTISSLQAEDVAVYYCQQNYDVPWTFGQGTKV | |
| | EIKRTVAAPSVFIFPPSDEQLKSGTASVVCLLNNFY | |
| | PREAKVQWKVDNALQSGNSQESVTEQDSKDSTYSLS | |
| | STLTLSKADYEKHKVYACEVTHQGLSSPVTKSFNRG | |
| | EC | |
| PD1 376 IgG-huIL2v T51P | EVQLLESGGGLVQPGGSLRLSCAASGFSFSSYTMSW | 22 |
| (P1AE0492) - HC hole | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVCTLPPSRDE | |
| | LTKNQVSLSCAVKGFYPSDIAVEWESNGQPENNYKT | |
| | TPPVLDSDGSFFLVSKLTVDKSRWQQGNVFSCSVMH | |
| | EALHNHYTQKSLSLSPGK | |
| PD1 376 IgG-huIL2v T51P | EVQLLESGGGLVQPGGSLRLSCAASGFSFSSYTMSW | 27 |
| (P1AE0492) - HC knob | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVYTLPPCRDE | |
| | LTKNQVSLWCLVKGFYPSDIAVEWESNGQPENNYKT | |
| | TPPVLDSDGSFFLYSKLTVDKSRWQQGNVFSCSVMH | |
| | EALHNHYTQKSLSLSPGGGGGSGGGGSGGGGSAPAS | |
| | SSTKKTQLQLEHLLLDLQMILNGINNYKNPKLTRML | |
| | TAKFAMPKKAPELKHLQCLEEELKPLEEVLNGAQSK | |
| | NFHLRPRDLISNINVIVLELKGSETTFMCEYADETA | |
| | TIVEFLNRWITFAQSIISTLT | |
| PD1 376 IgG-huIL2v H79S | DIVMTQSPDSLAVSLGERATINCKASESVDTSDNSF | 21 |
| (P1AE0493) - LC | IHWYQQKPGQSPKLLIYRSSTLESGVPDRFSGSGSG | |
| | TDFTLTISSLQAEDVAVYYCQQNYDVPWTFGQGTKV | |
| | EIKRTVAAPSVFIFPPSDEQLKSGTASVVCLLNNFY | |
| | PREAKVQWKVDNALQSGNSQESVTEQDSKDSTYSLS | |
| | STLTLSKADYEKHKVYACEVTHQGLSSPVTKSFNRG | |
| | EC | |
| PD1 376 IgG-huIL2v H79S | EVQLLESGGGLVQPGGSLRLSCAASGFSFSSYTMSW | 22 |
| (P1AE0493) - HC hole | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVCTLPPSRDE | |
| | LTKNQVSLSCAVKGFYPSDIAVEWESNGQPENNYKT | |
| | TPPVLDSDGSFFLVSKLTVDKSRWQQGNVFSCSVMH | |
| | EALHNHYTQKSLSLSPGK | |
| PD1 376 IgG-huIL2v H79S | EVQLLESGGGLVQPGGSLRLSCAASGFSFSSYTMSW | 28 |
| (P1AE0493) - HC knob | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVYTLPPCRDE | |
| | LTKNQVSLWCLVKGFYPSDIAVEWESNGQPENNYKT | |
| | TPPVLDSDGSFFLYSKLTVDKSRWQQGNVFSCSVMH | |
| | EALHNHYTQKSLSLSPGGGGGSGGGGSGGGGSAPAS | |
| | SSTKKTQLQLEHLLLDLQMILNGINNYKNPKLTRML | |
| | TAKFAMPKKATELKHLQCLEEELKPLEEVLNGAQSK | |
| | NFSLRPRDLISNINVIVLELKGSETTFMCEYADETA | |
| | TIVEFLNRWITFAQSIISTLT | |
| PD1 376 IgG-huIL2v L80A | DIVMTQSPDSLAVSLGERATINCKASESVDTSDNSF | 21 |
| (P1AE0494) - LC | IHWYQQKPGQSPKLLIYRSSTLESGVPDRESGSGSG | |
| | TDFTLTISSLQAEDVAVYYCQQNYDVPWTFGQGTKV | |
| | EIKRTVAAPSVFIFPPSDEQLKSGTASVVCLLNNFY | |
| | PREAKVQWKVDNALQSGNSQESVTEQDSKDSTYSLS | |
| | STLTLSKADYEKHKVYACEVTHQGLSSPVTKSFNRG | |
| | EC | |
| PD1 376 IgG-huIL2v L80A | EVQLLESGGGLVQPGGSLRLSCAASGFSFSSYTMSW | 22 |
| (P1AE0494) - HC hole | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVCTLPPSRDE | |
| | LTKNQVSLSCAVKGFYPSDIAVEWESNGQPENNYKT | |
| | TPPVLDSDGSFFLVSKLTVDKSRWQQGNVFSCSVMH | |
| | EALHNHYTQKSLSLSPGK | |
| PD1 376 IgG-huIL2v L80A | EVQLLESGGGLVQPGGSLRLSCAASGFSFSSYTMSW | 29 |
| (P1AE0494) - HC knob | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVYTLPPCRDE | |
| | LTKNQVSLWCLVKGFYPSDIAVEWESNGQPENNYKT | |
| | TPPVLDSDGSFFLYSKLTVDKSRWQQGNVFSCSVMH | |
| | EALHNHYTQKSLSLSPGGGGGSGGGGSGGGGSAPAS | |
| | SSTKKTQLQLEHLLLDLQMILNGINNYKNPKLTRML | |
| | TAKFAMPKKATELKHLQCLEEELKPLEEVLNGAQSK | |
| | NFHARPRDLISNINVIVLELKGSETTFMCEYADETA | |
| | TIVEFLNRWITFAQSIISTLT | |
| PD1 376 IgG-huIL2v N88A | DIVMTQSPDSLAVSLGERATINCKASESVDTSDNSF | 21 |
| (P1AE0495) - LC | IHWYQQKPGQSPKLLIYRSSTLESGVPDRFSGSGSG | |
| | TDFTLTISSLQAEDVAVYYCQQNYDVPWTFGQGTKV | |
| | EIKRTVAAPSVFIFPPSDEQLKSGTASVVCLLNNFY | |
| | PREAKVQWKVDNALQSGNSQESVTEQDSKDSTYSLS | |
| | STLTLSKADYEKHKVYACEVTHQGLSSPVTKSFNRG | |
| | EC | |
| PD1 376 IgG-huIL2v N88A | EVQLLESGGGLVQPGGSLRLSCAASGFSFSSYTMSW | 22 |
| (P1AE0495) - HC hole | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVCTLPPSRDE | |
| | LTKNQVSLSCAVKGFYPSDIAVEWESNGQPENNYKT | |
| | TPPVLDSDGSFFLVSKLTVDKSRWQQGNVFSCSVMH | |
| | EALHNHYTQKSLSLSPGK | |
| PD1 376 IgG-huIL2v N88A | EVQLLESGGGLVQPGGSLRLSCAASGFSFSSYTMSW | 30 |
| (P1AE0495) - HC knob | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVYTLPPCRDE | |
| | LTKNQVSLWCLVKGFYPSDIAVEWESNGQPENNYKT | |
| | TPPVLDSDGSFFLYSKLTVDKSRWQQGNVFSCSVMH | |
| | EALHNHYTQKSLSLSPGGGGGSGGGGSGGGGSAPAS | |
| | SSTKKTQLQLEHLLLDLQMILNGINNYKNPKLTRML | |
| | TAKFAMPKKATELKHLQCLEEELKPLEEVLNGAQSK | |
| | NFHLRPRDLISAINVIVLELKGSETTFMCEYADETA | |
| | TIVEFLNRWITFAQSIISTLT | |
| PD1 376 IgG-huIL2v N88Q | DIVMTQSPDSLAVSLGERATINCKASESVDTSDNSF | 21 |
| (P1AE0496) - LC | IHWYQQKPGQSPKLLIYRSSTLESGVPDRFSGSGSG | |
| | TDFTLTISSLQAEDVAVYYCQQNYDVPWTFGQGTKV | |
| | EIKRTVAAPSVFIFPPSDEQLKSGTASVVCLLNNFY | |
| | PREAKVQWKVDNALQSGNSQESVTEQDSKDSTYSLS | |
| | STLTLSKADYEKHKVYACEVTHQGLSSPVTKSFNRG | |
| | EC | |
| PD1 376 IgG-huIL2v N88Q | EVQLLESGGGLVQPGGSLRLSCAASGFSFSSYTMSW | 22 |
| (P1AE0496) - HC hole | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVCTLPPSRDE | |
| | LTKNQVSLSCAVKGFYPSDIAVEWESNGQPENNYKT | |
| | TPPVLDSDGSFFLVSKLTVDKSRWQQGNVFSCSVMH | |
| | EALHNHYTQKSLSLSPGK | |
| PD1 376 IgG-huIL2v N88Q | EVQLLESGGGLVQPGGSLRLSCAASGFSFSSYTMSW | 31 |
| (P1AE0496) - HC knob | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVYTLPPCRDE | |
| | LTKNQVSLWCLVKGFYPSDIAVEWESNGQPENNYKT | |
| | TPPVLDSDGSFFLYSKLTVDKSRWQQGNVFSCSVMH | |
| | EALHNHYTQKSLSLSPGGGGGSGGGGSGGGGSAPAS | |
| | SSTKKTQLQLEHLLLDLQMILNGINNYKNPKLTRML | |
| | TAKFAMPKKATELKHLQCLEEELKPLEEVLNGAQSK | |
| | NFHLRPRDLISQINVIVLELKGSETTFMCEYADETA | |
| | TIVEFLNRWITFAQSIISTLT | |
| PD1 376 IgG-huIL2v D109A | DIVMTQSPDSLAVSLGERATINCKASESVDTSDNSF | 21 |
| (P1AE0497) - LC | IHWYQQKPGQSPKLLIYRSSTLESGVPDRFSGSGSG | |
| | TDFTLTISSLQAEDVAVYYCQQNYDVPWTFGQGTKV | |
| | EIKRTVAAPSVFIFPPSDEQLKSGTASVVCLLNNFY | |
| | PREAKVQWKVDNALQSGNSQESVTEQDSKDSTYSLS | |
| | STLTLSKADYEKHKVYACEVTHQGLSSPVTKSFNRG | |
| | EC | |
| PD1 376 IgG-huIL2v D109A | EVQLLESGGGLVQPGGSLRLSCAASGFSFSSYTMSW | 22 |
| (P1AE0497) - HC hole | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVCTLPPSRDE | |
| | LTKNQVSLSCAVKGFYPSDIAVEWESNGQPENNYKT | |
| | TPPVLDSDGSFFLVSKLTVDKSRWQQGNVFSCSVMH | |
| | EALHNHYTQKSLSLSPGK | |
| PD1 376 IgG-huIL2v D109A | EVQLLESGGGLVQPGGSLRLSCAASGFSFSSYTMSW | 32 |
| (P1AE0497) - HC knob | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVYTLPPCRDE | |
| | LTKNQVSLWCLVKGFYPSDIAVEWESNGQPENNYKT | |
| | TPPVLDSDGSFFLYSKLTVDKSRWQQGNVFSCSVMH | |
| | EALHNHYTQKSLSLSPGGGGGSGGGGSGGGGSAPAS | |
| | SSTKKTQLQLEHLLLDLQMILNGINNYKNPKLTRML | |
| | TAKFAMPKKATELKHLQCLEEELKPLEEVLNGAQSK | |
| | NFHLRPRDLISNINVIVLELKGSETTFMCEYAAETA | |
| | TIVEFLNRWITFAQSIISTLT | |
| PD1 376 IgG-huIL2v Q126N | DIVMTQSPDSLAVSLGERATINCKASESVDTSDNSF | 21 |
| (P1AE0498) - LC | IHWYQQKPGQSPKLLIYRSSTLESGVPDRFSGSGSG | |
| | TDFTLTISSLQAEDVAVYYCQQNYDVPWTFGQGTKV | |
| | EIKRTVAAPSVFIFPPSDEQLKSGTASVVCLLNNFY | |
| | PREAKVQWKVDNALQSGNSQESVTEQDSKDSTYSLS | |
| | STLTLSKADYEKHKVYACEVTHQGLSSPVTKSFNRG | |
| | EC | |
| PD1 376 IgG-huIL2v Q126N | EVQLLESGGGLVQPGGSLRLSCAASGFSFSSYTMSW | 22 |
| (P1AE0498) - HC hole | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVCTLPPSRDE | |
| | LTKNQVSLSCAVKGFYPSDIAVEWESNGQPENNYKT | |
| | TPPVLDSDGSFFLVSKLTVDKSRWQQGNVFSCSVMH | |
| | EALHNHYTQKSLSLSPGK | |
| PD1 376 IgG-huIL2v Q126N | EVQLLESGGGLVQPGGSLRLSCAASGFSFSSYTMSW | 33 |
| (P1AE0498) - HC knob | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVYTLPPCRDE | |
| | LTKNQVSLWCLVKGFYPSDIAVEWESNGQPENNYKT | |
| | TPPVLDSDGSFFLYSKLTVDKSRWQQGNVFSCSVMH | |
| | EALHNHYTQKSLSLSPGGGGGSGGGGSGGGGSAPAS | |
| | SSTKKTQLQLEHLLLDLQMILNGINNYKNPKLTRML | |
| | TAKFAMPKKATELKHLQCLEEELKPLEEVLNGAQSK | |
| | NFHLRPRDLISNINVIVLELKGSETTFMCEYADETA | |
| | TIVEFLNRWITFANSIISTLT | |
| PD1 376 IgG-huIL2v Q126E | DIVMTQSPDSLAVSLGERATINCKASESVDTSDNSF | 21 |
| (P1AE0499) - LC | IHWYQQKPGQSPKLLIYRSSTLESGVPDRFSGSGSG | |
| | TDFTLTISSLQAEDVAVYYCQQNYDVPWTFGQGTKV | |
| | EIKRTVAAPSVFIFPPSDEQLKSGTASVVCLLNNFY | |
| | PREAKVQWKVDNALQSGNSQESVTEQDSKDSTYSLS | |
| | STLTLSKADYEKHKVYACEVTHQGLSSPVTKSFNRG | |
| | EC | |
| PD1 376 IgG-huIL2v Q126E | EVQLLESGGGLVQPGGSLRLSCAASGFSFSSYTMSW | 22 |
| (P1AE0499) - HC hole | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVCTLPPSRDE | |
| | LTKNQVSLSCAVKGFYPSDIAVEWESNGQPENNYKT | |
| | TPPVLDSDGSFFLVSKLTVDKSRWQQGNVFSCSVMH | |
| | EALHNHYTQKSLSLSPGK | |
| PD1 376 IgG-huIL2v Q126E | EVQLLESGGGLVQPGGSLRLSCAASGFSFSSYTMSW | 34 |
| (P1AE0499) - HC knob | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVYTLPPCRDE | |
| | LTKNQVSLWCLVKGFYPSDIAVEWESNGQPENNYKT | |
| | TPPVLDSDGSFFLYSKLTVDKSRWQQGNVFSCSVMH | |
| | EALHNHYTQKSLSLSPGGGGGSGGGGSGGGGSAPAS | |
| | SSTKKTQLQLEHLLLDLQMILNGINNYKNPKLTRML | |
| | TAKFAMPKKATELKHLQCLEEELKPLEEVLNGAQSK | |
| | NFHLRPRDLISNINVIVLELKGSETTFMCEYADETA | |
| | TIVEFLNRWITFAESIISTLT | |
| PD1 376 IgG-huIL2v Q126T | DIVMTQSPDSLAVSLGERATINCKASESVDTSDNSF | 21 |
| (P1AF2142) - LC | IHWYQQKPGQSPKLLIYRSSTLESGVPDRFSGSGSG | |
| | TDFTLTISSLQAEDVAVYYCQQNYDVPWTFGQGTKV | |
| | EIKRTVAAPSVFIFPPSDEQLKSGTASVVCLLNNFY | |
| | PREAKVQWKVDNALQSGNSQESVTEQDSKDSTYSLS | |
| | STLTLSKADYEKHKVYACEVTHQGLSSPVTKSFNRG | |
| | EC | |
| PD1 376 IgG-huIL2v Q126T | EVQLLESGGGLVQPGGSLRLSCAASGFSFSSYTMSW | 22 |
| (P1AF2142) - HC hole | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVCTLPPSRDE | |
| | LTKNQVSLSCAVKGFYPSDIAVEWESNGQPENNYKT | |
| | TPPVLDSDGSFFLVSKLTVDKSRWQQGNVFSCSVMH | |
| | EALHNHYTQKSLSLSPGK | |
| PD1 376 IgG-huIL2v Q126T | EVQLLESGGGLVQPGGSLRLSCAASGFSFSSYTMSW | 35 |
| (P1AF2142) - HC knob | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVYTLPPCRDE | |
| | LTKNQVSLWCLVKGFYPSDIAVEWESNGQPENNYKT | |
| | TPPVLDSDGSFFLYSKLTVDKSRWQQGNVFSCSVMH | |
| | EALHNHYTQKSLSLSPGGGGGSGGGGSGGGGSAPAS | |
| | SSTKKTQLQLEHLLLDLQMILNGINNYKNPKLTRML | |
| | TAKFAMPKKATELKHLQCLEEELKPLEEVLNGAQSK | |
| | NFHLRPRDLISNINVIVLELKGSETTFMCEYADETA | |
| | TIVEFLNRWITFATSIISTLT | |
| FAP 4B9 IgG-huIL2v_E15V | EIVLTQSPGTLSLSPGERATLSCRASQSVTSSYLAW | 1 |
| (P1AE4800) - LC | YQQKPGQAPRLLINVGSRRATGIPDRFSGSGSGTDF | |
| | TLTISRLEPEDFAVYYCQQGIMLPPTFGQGTKVEIK | |
| | RTVAAPSVFIFPPSDEQLKSGTASVVCLLNNFYPRE | |
| | AKVQWKVDNALQSGNSQESVTEQDSKDSTYSLSSTL | |
| | TLSKADYEKHKVYACEVTHQGLSSPVTKSFNRGEC | |
| FAP 4B9 IgG-huIL2v_E15V | EVQLLESGGGLVQPGGSLRLSCAASGFTFSSYAMSW | 2 |
| (P1AE4800) - HC hole | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVCTLPPSRDELTK | |
| | NQVSLSCAVKGFYPSDIAVEWESNGQPENNYKTTPP | |
| | VLDSDGSFFLVSKLTVDKSRWQQGNVFSCSVMHEAL | |
| | HNHYTQKSLSLSPGK | |
| FAP 4B9 IgG-huIL2v_E15V | EVQLLESGGGLVQPGGSLRLSCAASGFTFSSYAMSW | 36 |
| (P1AE4800) - HC knob | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVYTLPPCRDELTK | |
| | NQVSLWCLVKGFYPSDIAVEWESNGQPENNYKTTPP | |
| | VLDSDGSFFLYSKLTVDKSRWQQGNVFSCSVMHEAL | |
| | HNHYTQKSLSLSPGGGGGSGGGGSGGGGSGGGGSGG | |
| | GGSAPASSSTKKTQLQLVHLLLDLQMILNGINNYKN | |
| | PKLTRMLTAKFAMPKKATELKHLQCLEEELKPLEEV | |
| | LNGAQSKNFHLRPRDLISNINVIVLELKGSETTFMC | |
| | EYADETATIVEFLNRWITFAQSIISTLT | |
| FAP 4B9 IgG-huIL2v_E95A | EIVLTQSPGTLSLSPGERATLSCRASQSVTSSYLAW | 1 |
| (P1AE4801) - LC | YQQKPGQAPRLLINVGSRRATGIPDRFSGSGSGTDF | |
| | TLTISRLEPEDFAVYYCQQGIMLPPTFGQGTKVEIK | |
| | RTVAAPSVFIFPPSDEQLKSGTASVVCLLNNFYPRE | |
| | AKVQWKVDNALQSGNSQESVTEQDSKDSTYSLSSTL | |
| | TLSKADYEKHKVYACEVTHQGLSSPVTKSFNRGEC | |
| FAP 4B9 IgG-huIL2v_E95A | EVQLLESGGGLVQPGGSLRLSCAASGFTFSSYAMSW | 2 |
| (P1AE4801) - HC hole | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVCTLPPSRDELTK | |
| | NQVSLSCAVKGFYPSDIAVEWESNGQPENNYKTTPP | |
| | VLDSDGSFFLVSKLTVDKSRWQQGNVFSCSVMHEAL | |
| | HNHYTQKSLSLSPGK | |
| FAP 4B9 IgG-huIL2v_E95A | EVQLLESGGGLVQPGGSLRLSCAASGFTFSSYAMSW | 37 |
| (P1AE4801) - HC knob | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVYTLPPCRDELTK | |
| | NQVSLWCLVKGFYPSDIAVEWESNGQPENNYKTTPP | |
| | VLDSDGSFFLYSKLTVDKSRWQQGNVFSCSVMHEAL | |
| | HNHYTQKSLSLSPGGGGGSGGGGSGGGGSGGGGSGG | |
| | GGSAPASSSTKKTQLQLEHLLLDLQMILNGINNYKN | |
| | PKLTRMLTAKFAMPKKATELKHLQCLEEELKPLEEV | |
| | LNGAQSKNFHLRPRDLISNINVIVLALKGSETTFMC | |
| | EYADETATIVEFLNRWITFAQSIISTLT | |
| FAP 4B9 IgG-huIL2v_L12A | EIVLTQSPGTLSLSPGERATLSCRASQSVTSSYLAW | 1 |
| (P1AE4804) - LC | YQQKPGQAPRLLINVGSRRATGIPDRFSGSGSGTDF | |
| | TLTISRLEPEDFAVYYCQQGIMLPPTFGQGTKVEIK | |
| | RTVAAPSVFIFPPSDEQLKSGTASVVCLLNNFYPRE | |
| | AKVQWKVDNALQSGNSQESVTEQDSKDSTYSLSSTL | |
| | TLSKADYEKHKVYACEVTHQGLSSPVTKSFNRGEC | |
| FAP 4B9 IgG-huIL2v_L12A | EVQLLESGGGLVQPGGSLRLSCAASGFTFSSYAMSW | 2 |
| (P1AE4804) - HC hole | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVCTLPPSRDELTK | |
| | NQVSLSCAVKGFYPSDIAVEWESNGQPENNYKTTPP | |
| | VLDSDGSFFLVSKLTVDKSRWQQGNVFSCSVMHEAL | |
| | HNHYTQKSLSLSPGK | |
| FAP 4B9 IgG-huIL2v_L12A | EVQLLESGGGLVQPGGSLRLSCAASGFTFSSYAMSW | 38 |
| (P1AE4804) - HC knob | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVYTLPPCRDELTK | |
| | NQVSLWCLVKGFYPSDIAVEWESNGQPENNYKTTPP | |
| | VLDSDGSFFLYSKLTVDKSRWQQGNVFSCSVMHEAL | |
| | HNHYTQKSLSLSPGGGGGSGGGGSGGGGSGGGGSGG | |
| | GGSAPASSSTKKTQAQLEHLLLDLQMILNGINNYKN | |
| | PKLTRMLTAKFAMPKKATELKHLQCLEEELKPLEEV | |
| | LNGAQSKNFHLRPRDLISNINVIVLELKGSETTFMC | |
| | EYADETATIVEFLNRWITFAQSIISTLT | |
| FAP 4B9 IgG- | EIVLTQSPGTLSLSPGERATLSCRASQSVTSSYLAW | 1 |
| huIL2v_L12A_L19A | YQQKPGQAPRLLINVGSRRATGIPDRFSGSGSGTDF | |
| (P1AE4806) - LC | TLTISRLEPEDFAVYYCQQGIMLPPTFGQGTKVEIK | |
| | RTVAAPSVFIFPPSDEQLKSGTASVVCLLNNFYPRE | |
| | AKVQWKVDNALQSGNSQESVTEQDSKDSTYSLSSTL | |
| | TLSKADYEKHKVYACEVTHQGLSSPVTKSFNRGEC | |
| FAP 4B9 IgG- | EVQLLESGGGLVQPGGSLRLSCAASGFTFSSYAMSW | 2 |
| huIL2v_L12A_L19A | VRQAPGKGLEWVSAIIGSGASTYYADSVKGRFTISR | |
| (P1AE4806) - HC hole | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVCTLPPSRDELTK | |
| | NQVSLSCAVKGFYPSDIAVEWESNGQPENNYKTTPP | |
| | VLDSDGSFFLVSKLTVDKSRWQQGNVFSCSVMHEAL | |
| | HNHYTQKSLSLSPGK | |
| FAP 4B9 IgG- | EVQLLESGGGLVQPGGSLRLSCAASGFTFSSYAMSW | 39 |
| huIL2v_L12A_L19A (P1AE4806) | VRQAPGKGLEWVSAIIGSGASTYYADSVKGRFTISR | |
| - HC knob | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVYTLPPCRDELTK | |
| | NQVSLWCLVKGFYPSDIAVEWESNGQPENNYKTTPP | |
| | VLDSDGSFFLYSKLTVDKSRWQQGNVFSCSVMHEAL | |
| | HNHYTQKSLSLSPGGGGGSGGGGSGGGGSGGGGSGG | |
| | GGSAPASSSTKKTQAQLEHLLADLQMILNGINNYKN | |
| | PKLTRMLTAKFAMPKKATELKHLQCLEEELKPLEEV | |
| | LNGAQSKNFHLRPRDLISNINVIVLELKGSETTFMC | |
| | EYADETATIVEFLNRWITFAQSIISTLT | |
| FAP 4B9 IgG- | EIVLTQSPGTLSLSPGERATLSCRASQSVTSSYLAW | 1 |
| huIL2v_L12A_L80A | YQQKPGQAPRLLINVGSRRATGIPDRFSGSGSGTDF | |
| (P1AE4807) - LC | TLTISRLEPEDFAVYYCQQGIMLPPTFGQGTKVEIK | |
| | RTVAAPSVFIFPPSDEQLKSGTASVVCLLNNFYPRE | |
| | AKVQWKVDNALQSGNSQESVTEQDSKDSTYSLSSTL | |
| | TLSKADYEKHKVYACEVTHQGLSSPVTKSFNRGEC | |
| FAP 4B9 IgG- | EVQLLESGGGLVQPGGSLRLSCAASGFTFSSYAMSW | 2 |
| huIL2v_L12A_L80A | VRQAPGKGLEWVSAIIGSGASTYYADSVKGRFTISR | |
| (P1AE4807) - HC hole | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVCTLPPSRDELTK | |
| | NQVSLSCAVKGFYPSDIAVEWESNGQPENNYKTTPP | |
| | VLDSDGSFFLVSKLTVDKSRWQQGNVFSCSVMHEAL | |
| | HNHYTQKSLSLSPGK | |
| FAP 4B9 IgG- | EVQLLESGGGLVQPGGSLRLSCAASGFTFSSYAMSW | 40 |
| huIL2v_L12A_L80A (P1AE4807) | VRQAPGKGLEWVSAIIGSGASTYYADSVKGRFTISR | |
| - HC knob | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVYTLPPCRDELTK | |
| | NQVSLWCLVKGFYPSDIAVEWESNGQPENNYKTTPP | |
| | VLDSDGSFFLYSKLTVDKSRWQQGNVFSCSVMHEAL | |
| | HNHYTQKSLSLSPGGGGGSGGGGSGGGGSGGGGSGG | |
| | GGSAPASSSTKKTQAQLEHLLLDLQMILNGINNYKN | |
| | PKLTRMLTAKFAMPKKATELKHLQCLEEELKPLEEV | |
| | LNGAQSKNFHARPRDLISNINVIVLELKGSETTFMC | |
| | EYADETATIVEFLNRWITFAQSIISTLT | |
| FAP 4B9 IgG-huIL2v_L19V | EIVLTQSPGTLSLSPGERATLSCRASQSVTSSYLAW | 1 |
| (P1AE4808) - LC | YQQKPGQAPRLLINVGSRRATGIPDRFSGSGSGTDF | |
| | TLTISRLEPEDFAVYYCQQGIMLPPTFGQGTKVEIK | |
| | RTVAAPSVFIFPPSDEQLKSGTASVVCLLNNFYPRE | |
| | AKVQWKVDNALQSGNSQESVTEQDSKDSTYSLSSTL | |
| | TLSKADYEKHKVYACEVTHQGLSSPVTKSFNRGEC | |
| FAP 4B9 IgG-huIL2v_L19V | EVQLLESGGGLVQPGGSLRLSCAASGFTFSSYAMSW | 2 |
| (P1AE4808) - HC hole | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVCTLPPSRDELTK | |
| | NQVSLSCAVKGFYPSDIAVEWESNGQPENNYKTTPP | |
| | VLDSDGSFFLVSKLTVDKSRWQQGNVFSCSVMHEAL | |
| | HNHYTQKSLSLSPGK | |
| FAP 4B9 IgG-huIL2v_L19V | EVQLLESGGGLVQPGGSLRLSCAASGFTFSSYAMSW | 41 |
| (P1AE4808) - HC knob | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVYTLPPCRDELTK | |
| | NQVSLWCLVKGFYPSDIAVEWESNGQPENNYKTTPP | |
| | VLDSDGSFFLYSKLTVDKSRWQQGNVFSCSVMHEAL | |
| | HNHYTQKSLSLSPGGGGGSGGGGSGGGGSGGGGSGG | |
| | GGSAPASSSTKKTQLQLEHLLVDLQMILNGINNYKN | |
| | PKLTRMLTAKFAMPKKATELKHLQCLEEELKPLEEV | |
| | LNGAQSKNFHLRPRDLISNINVIVLELKGSETTFMC | |
| | EYADETATIVEFLNRWITFAQSIISTLT | |
| FAP 4B9 IgG-huIL2v_N88T | EIVLTQSPGTLSLSPGERATLSCRASQSVTSSYLAW | 1 |
| (P1AE4809) - LC | YQQKPGQAPRLLINVGSRRATGIPDRESGSGSGTDF | |
| | TLTISRLEPEDFAVYYCQQGIMLPPTFGQGTKVEIK | |
| | RTVAAPSVFIFPPSDEQLKSGTASVVCLLNNFYPRE | |
| | AKVQWKVDNALQSGNSQESVTEQDSKDSTYSLSSTL | |
| | TLSKADYEKHKVYACEVTHQGLSSPVTKSFNRGEC | |
| FAP 4B9 IgG-huIL2v_N88T | EVQLLESGGGLVQPGGSLRLSCAASGFTFSSYAMSW | 2 |
| (P1AE4809) - HC hole | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVCTLPPSRDELTK | |
| | NQVSLSCAVKGFYPSDIAVEWESNGQPENNYKTTPP | |
| | VLDSDGSFFLVSKLTVDKSRWQQGNVFSCSVMHEAL | |
| | HNHYTQKSLSLSPGK | |
| FAP 4B9 IgG-huIL2v_N88T | EVQLLESGGGLVQPGGSLRLSCAASGFTFSSYAMSW | 42 |
| (P1AE4809) - HC knob | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVYTLPPCRDELTK | |
| | NQVSLWCLVKGFYPSDIAVEWESNGQPENNYKTTPP | |
| | VLDSDGSFFLYSKLTVDKSRWQQGNVFSCSVMHEAL | |
| | HNHYTQKSLSLSPGGGGGSGGGGSGGGGSGGGGSGG | |
| | GGSAPASSSTKKTQLQLEHLLLDLQMILNGINNYKN | |
| | PKLTRMLTAKFAMPKKATELKHLQCLEEELKPLEEV | |
| | LNGAQSKNFHLRPRDLISTINVIVLELKGSETTFMC | |
| | EYADETATIVEFLNRWITFAQSIISTLT | |
| FAP 4B9 IgG-huIL2v_N119K | EIVLTQSPGTLSLSPGERATLSCRASQSVTSSYLAW | 1 |
| (P1AE4810) - LC | YQQKPGQAPRLLINVGSRRATGIPDRFSGSGSGTDF | |
| | TLTISRLEPEDFAVYYCQQGIMLPPTFGQGTKVEIK | |
| | RTVAAPSVFIFPPSDEQLKSGTASVVCLLNNFYPRE | |
| | AKVQWKVDNALQSGNSQESVTEQDSKDSTYSLSSTL | |
| | TLSKADYEKHKVYACEVTHQGLSSPVTKSFNRGEC | |
| FAP 4B9 IgG-huIL2v_N119K | EVQLLESGGGLVQPGGSLRLSCAASGFTFSSYAMSW | 2 |
| (P1AE4810) - HC hole | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKENWYVDGVEVHNAK | |
| | TKPREEQYNSTYRVVSVLTVLHQDWLNGKEYKCKVS | |
| | NKALGAPIEKTISKAKGQPREPQVCTLPPSRDELTK | |
| | NQVSLSCAVKGFYPSDIAVEWESNGQPENNYKTTPP | |
| | VLDSDGSFFLVSKLTVDKSRWQQGNVFSCSVMHEAL | |
| | HNHYTQKSLSLSPGK | |
| FAP 4B9 IgG-huIL2v_N119K | EVQLLESGGGLVQPGGSLRLSCAASGFTFSSYAMSW | 43 |
| (P1AE4810) - HC knob | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVYTLPPCRDELTK | |
| | NQVSLWCLVKGFYPSDIAVEWESNGQPENNYKTTPP | |
| | VLDSDGSFFLYSKLTVDKSRWQQGNVFSCSVMHEAL | |
| | HNHYTQKSLSLSPGGGGGSGGGGSGGGGSGGGGSGG | |
| | GGSAPASSSTKKTQLQLEHLLLDLQMILNGINNYKN | |
| | PKLTRMLTAKFAMPKKATELKHLQCLEEELKPLEEV | |
| | LNGAQSKNFHLRPRDLISNINVIVLELKGSETTFMC | |
| | EYADETATIVEFLKRWITFAQSIISTLT | |
| FAP 4B9 IgG-huIL2v_Q22A | EIVLTQSPGTLSLSPGERATLSCRASQSVTSSYLAW | 1 |
| (P1AE4811) - LC | YQQKPGQAPRLLINVGSRRATGIPDRFSGSGSGTDF | |
| | TLTISRLEPEDFAVYYCQQGIMLPPTFGQGTKVEIK | |
| | RTVAAPSVFIFPPSDEQLKSGTASVVCLLNNFYPRE | |
| | AKVQWKVDNALQSGNSQESVTEQDSKDSTYSLSSTL | |
| | TLSKADYEKHKVYACEVTHQGLSSPVTKSFNRGEC | |
| FAP 4B9 IgG-huIL2v_Q22A | EVQLLESGGGLVQPGGSLRLSCAASGFTFSSYAMSW | 2 |
| (P1AE4811) - HC hole | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVCTLPPSRDELTK | |
| | NQVSLSCAVKGFYPSDIAVEWESNGQPENNYKTTPP | |
| | VLDSDGSFFLVSKLTVDKSRWQQGNVFSCSVMHEAL | |
| | HNHYTQKSLSLSPGK | |
| FAP 4B9 IgG-huIL2v_Q22A | EVQLLESGGGLVQPGGSLRLSCAASGFTFSSYAMSW | 44 |
| (P1AE4811) - HC knob | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVYTLPPCRDELTK | |
| | NQVSLWCLVKGFYPSDIAVEWESNGQPENNYKTTPP | |
| | VLDSDGSFFLYSKLTVDKSRWQQGNVFSCSVMHEAL | |
| | HNHYTQKSLSLSPGGGGGSGGGGSGGGGSGGGGSGG | |
| | GGSAPASSSTKKTQLQLEHLLLDLAMILNGINNYKN | |
| | PKLTRMLTAKFAMPKKATELKHLQCLEEELKPLEEV | |
| | LNGAQSKNFHLRPRDLISNINVIVLELKGSETTFMC | |
| | EYADETATIVEFLNRWITFAQSIISTLT | |
| FAP 4B9 IgG-huIL2v_Q126T | EIVLTQSPGTLSLSPGERATLSCRASQSVTSSYLAW | 1 |
| (P1AE4812) - LC | YQQKPGQAPRLLINVGSRRATGIPDRFSGSGSGTDF | |
| | TLTISRLEPEDFAVYYCQQGIMLPPTFGQGTKVEIK | |
| | RTVAAPSVFIFPPSDEQLKSGTASVVCLLNNFYPRE | |
| | AKVQWKVDNALQSGNSQESVTEQDSKDSTYSLSSTL | |
| | TLSKADYEKHKVYACEVTHQGLSSPVTKSFNRGEC | |
| FAP 4B9 IgG-huIL2v_Q126T | EVQLLESGGGLVQPGGSLRLSCAASGFTFSSYAMSW | 2 |
| (P1AE4812) - HC hole | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVCTLPPSRDELTK | |
| | NQVSLSCAVKGFYPSDIAVEWESNGQPENNYKTTPP | |
| | VLDSDGSFFLVSKLTVDKSRWQQGNVFSCSVMHEAL | |
| | HNHYTQKSLSLSPGK | |
| FAP 4B9 IgG-huIL2v_Q126T | EVQLLESGGGLVQPGGSLRLSCAASGFTFSSYAMSW | 45 |
| (P1AE4812) - HC knob | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVYTLPPCRDELTK | |
| | NQVSLWCLVKGFYPSDIAVEWESNGQPENNYKTTPP | |
| | VLDSDGSFFLYSKLTVDKSRWQQGNVFSCSVMHEAL | |
| | HNHYTQKSLSLSPGGGGGSGGGGSGGGGSGGGGSGG | |
| | GGSAPASSSTKKTQLQLEHLLLDLQMILNGINNYKN | |
| | PKLTRMLTAKFAMPKKATELKHLQCLEEELKPLEEV | |
| | LNGAQSKNFHLRPRDLISNINVIVLELKGSETTFMC | |
| | EYADETATIVEFLNRWITFATSIISTLT | |
| FAP 4B9 IgG-huIL2v_S87A | EIVLTQSPGTLSLSPGERATLSCRASQSVTSSYLAW | 1 |
| (P1AE4813) - LC | YQQKPGQAPRLLINVGSRRATGIPDRFSGSGSGTDF | |
| | TLTISRLEPEDFAVYYCQQGIMLPPTFGQGTKVEIK | |
| | RTVAAPSVFIFPPSDEQLKSGTASVVCLLNNFYPRE | |
| | AKVQWKVDNALQSGNSQESVTEQDSKDSTYSLSSTL | |
| | TLSKADYEKHKVYACEVTHQGLSSPVTKSFNRGEC | |
| FAP 4B9 IgG-huIL2v_S87A | EVQLLESGGGLVQPGGSLRLSCAASGFTFSSYAMSW | 2 |
| (P1AE4813) - HC hole | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVCTLPPSRDELTK | |
| | NQVSLSCAVKGFYPSDIAVEWESNGQPENNYKTTPP | |
| | VLDSDGSFFLVSKLTVDKSRWQQGNVFSCSVMHEAL | |
| | HNHYTQKSLSLSPGK | |
| FAP 4B9 IgG-huIL2v_S87A | EVQLLESGGGLVQPGGSLRLSCAASGFTFSSYAMSW | 46 |
| (P1AE4813) - HC knob | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVYTLPPCRDELTK | |
| | NQVSLWCLVKGFYPSDIAVEWESNGQPENNYKTTPP | |
| | VLDSDGSFFLYSKLTVDKSRWQQGNVFSCSVMHEAL | |
| | HNHYTQKSLSLSPGGGGGSGGGGSGGGGSGGGGSGG | |
| | GGSAPASSSTKKTQLQLEHLLLDLQMILNGINNYKN | |
| | PKLTRMLTAKFAMPKKATELKHLQCLEEELKPLEEV | |
| | LNGAQSKNFHLRPRDLIANINVIVLELKGSETTFMC | |
| | EYADETATIVEFLNRWITFAQSIISTLT | |
| FAP 4B9 IgG-huIL2v_S130A | EIVLTQSPGTLSLSPGERATLSCRASQSVTSSYLAW | 1 |
| (P1AE4814) - LC | YQQKPGQAPRLLINVGSRRATGIPDRFSGSGSGTDF | |
| | TLTISRLEPEDFAVYYCQQGIMLPPTFGQGTKVEIK | |
| | RTVAAPSVFIFPPSDEQLKSGTASVVCLLNNFYPRE | |
| | AKVQWKVDNALQSGNSQESVTEQDSKDSTYSLSSTL | |
| | TLSKADYEKHKVYACEVTHQGLSSPVTKSFNRGEC | |
| FAP 4B9 IgG-huIL2v_S130A | EVQLLESGGGLVQPGGSLRLSCAASGFTFSSYAMSW | 2 |
| (P1AE4814) - HC hole | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVCTLPPSRDELTK | |
| | NQVSLSCAVKGFYPSDIAVEWESNGQPENNYKTTPP | |
| | VLDSDGSFFLVSKLTVDKSRWQQGNVFSCSVMHEAL | |
| | HNHYTQKSLSLSPGK | |
| FAP 4B9 IgG-huIL2v_S130A | EVQLLESGGGLVQPGGSLRLSCAASGFTFSSYAMSW | 47 |
| (P1AE4814) - HC knob | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVYTLPPCRDELTK | |
| | NQVSLWCLVKGFYPSDIAVEWESNGQPENNYKTTPP | |
| | VLDSDGSFFLYSKLTVDKSRWQQGNVFSCSVMHEAL | |
| | HNHYTQKSLSLSPGGGGGSGGGGSGGGGSGGGGSGG | |
| | GGSAPASSSTKKTQLQLEHLLLDLQMILNGINNYKN | |
| | PKLTRMLTAKFAMPKKATELKHLQCLEEELKPLEEV | |
| | LNGAQSKNFHLRPRDLISNINVIVLELKGSETTFMC | |
| | EYADETATIVEFLNRWITFAQSIIATLT | |
| FAP 4B9 IgG-huIL2v_T133K | EIVLTQSPGTLSLSPGERATLSCRASQSVTSSYLAW | 1 |
| (P1AE4816) - LC | YQQKPGQAPRLLINVGSRRATGIPDRESGSGSGTDF | |
| | TLTISRLEPEDFAVYYCQQGIMLPPTFGQGTKVEIK | |
| | RTVAAPSVFIFPPSDEQLKSGTASVVCLLNNFYPRE | |
| | AKVQWKVDNALQSGNSQESVTEQDSKDSTYSLSSTL | |
| | TLSKADYEKHKVYACEVTHQGLSSPVTKSFNRGEC | |
| FAP 4B9 IgG-huIL2v_T133K | EVQLLESGGGLVQPGGSLRLSCAASGFTFSSYAMSW | 2 |
| (P1AE4816) - HC hole | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVCTLPPSRDELTK | |
| | NQVSLSCAVKGFYPSDIAVEWESNGQPENNYKTTPP | |
| | VLDSDGSFFLVSKLTVDKSRWQQGNVFSCSVMHEAL | |
| | HNHYTQKSLSLSPGK | |
| FAP 4B9 IgG-huIL2v_T133K | EVQLLESGGGLVQPGGSLRLSCAASGFTFSSYAMSW | 48 |
| (P1AE4816) - HC knob | VRQAPGKGLEWVSAIIGSGASTYYADSVKGRFTISR | |
| | DNSKNTLYLQMNSLRAEDTAVYYCAKGWFGGFNYWG | |
| | QGTLVTVSSASTKGPSVFPLAPSSKSTSGGTAALGC | |
| | LVKDYFPEPVTVSWNSGALTSGVHTFPAVLQSSGLY | |
| | SLSSVVTVPSSSLGTQTYICNVNHKPSNTKVDKKVE | |
| | PKSCDKTHTCPPCPAPEAAGGPSVFLFPPKPKDTLM | |
| | ISRTPEVTCVVVDVSHEDPEVKFNWYVDGVEVHNAK | |
| | TKPREEQYNSTYRVVSVLTVLHQDWLNGKEYKCKVS | |
| | NKALGAPIEKTISKAKGQPREPQVYTLPPCRDELTK | |
| | NQVSLWCLVKGFYPSDIAVEWESNGQPENNYKTTPP | |
| | VLDSDGSFFLYSKLTVDKSRWQQGNVFSCSVMHEAL | |
| | HNHYTQKSLSLSPGGGGGSGGGGSGGGGSGGGGSGG | |
| | GGSAPASSSTKKTQLQLEHLLLDLQMILNGINNYKN | |
| | PKLTRMLTAKFAMPKKATELKHLQCLEEELKPLEEV | |
| | LNGAQSKNFHLRPRDLISNINVIVLELKGSETTFMC | |
| | EYADETATIVEFLNRWITFAQSIISTLK | |
| NKG2D-IL2v K8S alias NKG2D- | SSELTQDPAVSVALGQTVRITCQGDSLRSYYASWYQ | 49 |
| IL2QM-F-09-05 (P1AE2763) - LC | QKPGQAPVLVIYGKNNRPSGIPDRFSGSSSGNTASL | |
| | TITGAQAEDEADYYCNSRDSFSIHQNVFGGGTKLTV | |
| | LGQPKAAPSVTLFPPSSEELQANKATLVCLISDFYP | |
| | GAVTVAWKADSSPVKAGVETTTPSKQSNNKYAASSY | |
| | LSLTPEQWKSHRSYSCQVTHEGSTVEKTVAPTECS | |
| NKG2D-IL2v K8S alias NKG2D- | EVQLLESGGGLVQPGGSLRLSCAASGFTFSSYAMSW | 50 |
| IL2QM-F-09-05 (P1AE2763) - HC | VRQAPGKGLEWVSAISGSGGSTYYADSVKGRFTISR | |
| hole | DNSKNTLYLQMNSLRAEDTAVYYCAKELYREYMDYW | |
| | GQGTLVTVSSASTKGPSVFPLAPSSKSTSGGTAALG | |
| | CLVKDYFPEPVTVSWNSGALTSGVHTFPAVLQSSGL | |
| | YSLSSVVTVPSSSLGTQTYICNVNHKPSNTKVDKKV | |
| | EPKSCDKTHTCPPCPAPEAAGGPSVFLFPPKPKDTL | |
| | MISRTPEVTCVVVDVSHEDPEVKFNWYVDGVEVHNA | |
| | KTKPREEQYNSTYRVVSVLTVLHQDWLNGKEYKCKV | |
| | SNKALGAPIEKTISKAKGQPREPQVCTLPPSRDELT | |
| | KNQVSLSCAVKGFYPSDIAVEWESNGQPENNYKTTP | |
| | PVLDSDGSFFLVSKLTVDKSRWQQGNVFSCSVMHEA | |
| | LHNHYTQKSLSLSPGK | |
| NKG2D-IL2v K8S alias NKG2D- | EVQLLESGGGLVQPGGSLRLSCAASGFTFSSYAMSW | 51 |
| IL2QM-F-09-05 (P1AE2763) - HC | VRQAPGKGLEWVSAISGSGGSTYYADSVKGRFTISR | |
| knob | DNSKNTLYLQMNSLRAEDTAVYYCAKELYREYMDYW | |
| | GQGTLVTVSSASTKGPSVFPLAPSSKSTSGGTAALG | |
| | CLVKDYFPEPVTVSWNSGALTSGVHTFPAVLQSSGL | |
| | YSLSSVVTVPSSSLGTQTYICNVNHKPSNTKVDKKV | |
| | EPKSCDKTHTCPPCPAPEAAGGPSVFLFPPKPKDTL | |
| | MISRTPEVTCVVVDVSHEDPEVKFNWYVDGVEVHNA | |
| | KTKPREEQYNSTYRVVSVLTVLHQDWLNGKEYKCKV | |
| | SNKALGAPIEKTISKAKGQPREPQVYTLPPCRDELT | |
| | KNQVSLWCLVKGFYPSDIAVEWESNGQPENNYKTTP | |
| | PVLDSDGSFFLYSKLTVDKSRWQQGNVFSCSVMHEA | |
| | LHNHYTQKSLSLSPGGGGGSGGGGSGGGGSAPASSS | |
| | TSKTQLQLEHLLLDLQMILNGINNYKNPKLTRMLTA | |
| | KFAMPKKATELKHLQCLEEELKPLEEVLNGAQSKNF | |
| | HLRPRDLISNINVIVLELKGSETTFMCEYADETATI | |
| | VEFLNRWITFAQSIISTLT | |
| NKG2D-IL2v L12A alias NKG2D- | SSELTQDPAVSVALGQTVRITCQGDSLRSYYASWYQ | 49 |
| IL2QM-F-09-06 (P1AE2764) - LC | QKPGQAPVLVIYGKNNRPSGIPDRFSGSSSGNTASL | |
| | TITGAQAEDEADYYCNSRDSFSIHQNVFGGGTKLTV | |
| | LGQPKAAPSVTLFPPSSEELQANKATLVCLISDFYP | |
| | GAVTVAWKADSSPVKAGVETTTPSKQSNNKYAASSY | |
| | LSLTPEQWKSHRSYSCQVTHEGSTVEKTVAPTECS | |
| NKG2D-IL2v L12A alias NKG2D- | EVQLLESGGGLVQPGGSLRLSCAASGFTFSSYAMSW | 50 |
| IL2QM-F-09-06 (P1AE2764) - HC | VRQAPGKGLEWVSAISGSGGSTYYADSVKGRFTISR | |
| hole | DNSKNTLYLQMNSLRAEDTAVYYCAKELYREYMDYW | |
| | GQGTLVTVSSASTKGPSVFPLAPSSKSTSGGTAALG | |
| | CLVKDYFPEPVTVSWNSGALTSGVHTFPAVLQSSGL | |
| | YSLSSVVTVPSSSLGTQTYICNVNHKPSNTKVDKKV | |
| | EPKSCDKTHTCPPCPAPEAAGGPSVFLFPPKPKDTL | |
| | MISRTPEVTCVVVDVSHEDPEVKFNWYVDGVEVHNA | |
| | KTKPREEQYNSTYRVVSVLTVLHQDWLNGKEYKCKV | |
| | SNKALGAPIEKTISKAKGQPREPQVCTLPPSRDELT | |
| | KNQVSLSCAVKGFYPSDIAVEWESNGQPENNYKTTP | |
| | PVLDSDGSFFLVSKLTVDKSRWQQGNVFSCSVMHEA | |
| | LHNHYTQKSLSLSPGK | |
| NKG2D-IL2v L12A alias NKG2D- | EVQLLESGGGLVQPGGSLRLSCAASGFTFSSYAMSW | 52 |
| IL2QM-F-09-06 (P1AE2764) - HC | VRQAPGKGLEWVSAISGSGGSTYYADSVKGRFTISR | |
| knob | DNSKNTLYLQMNSLRAEDTAVYYCAKELYREYMDYW | |
| | GQGTLVTVSSASTKGPSVFPLAPSSKSTSGGTAALG | |
| | CLVKDYFPEPVTVSWNSGALTSGVHTFPAVLQSSGL | |
| | YSLSSVVTVPSSSLGTQTYICNVNHKPSNTKVDKKV | |
| | EPKSCDKTHTCPPCPAPEAAGGPSVFLFPPKPKDTL | |
| | MISRTPEVTCVVVDVSHEDPEVKFNWYVDGVEVHNA | |
| | KTKPREEQYNSTYRVVSVLTVLHQDWLNGKEYKCKV | |
| | SNKALGAPIEKTISKAKGQPREPQVYTLPPCRDELT | |
| | KNQVSLWCLVKGFYPSDIAVEWESNGQPENNYKTTP | |
| | PVLDSDGSFFLYSKLTVDKSRWQQGNVFSCSVMHEA | |
| | LHNHYTQKSLSLSPGGGGGSGGGGSGGGGSAPASSS | |
| | TKKTQAQLEHLLLDLQMILNGINNYKNPKLTRMLTA | |
| | KFAMPKKATELKHLQCLEEELKPLEEVLNGAQSKNF | |
| | HLRPRDLISNINVIVLELKGSETTFMCEYADETATI | |
| | VEFLNRWITFAQSIISTLT | |
| NKG2D-IL2v L19V alias NKG2D- | SSELTQDPAVSVALGQTVRITCQGDSLRSYYASWYQ | 49 |
| IL2QM-F-09-07 (P1AE2765) - LC | QKPGQAPVLVIYGKNNRPSGIPDRFSGSSSGNTASL | |
| | TITGAQAEDEADYYCNSRDSFSIHQNVFGGGTKLTV | |
| | LGQPKAAPSVTLFPPSSEELQANKATLVCLISDFYP | |
| | GAVTVAWKADSSPVKAGVETTTPSKQSNNKYAASSY | |
| | LSLTPEQWKSHRSYSCQVTHEGSTVEKTVAPTECS | |
| NKG2D-IL2v L19V alias NKG2D- | EVQLLESGGGLVQPGGSLRLSCAASGFTFSSYAMSW | 50 |
| IL2QM-F-09-07 (P1AE2765) - HC | VRQAPGKGLEWVSAISGSGGSTYYADSVKGRFTISR | |
| hole | DNSKNTLYLQMNSLRAEDTAVYYCAKELYREYMDYW | |
| | GQGTLVTVSSASTKGPSVFPLAPSSKSTSGGTAALG | |
| | CLVKDYFPEPVTVSWNSGALTSGVHTFPAVLQSSGL | |
| | YSLSSVVTVPSSSLGTQTYICNVNHKPSNTKVDKKV | |
| | EPKSCDKTHTCPPCPAPEAAGGPSVFLFPPKPKDTL | |
| | MISRTPEVTCVVVDVSHEDPEVKFNWYVDGVEVHNA | |
| | KTKPREEQYNSTYRVVSVLTVLHQDWLNGKEYKCKV | |
| | SNKALGAPIEKTISKAKGQPREPQVCTLPPSRDELT | |
| | KNQVSLSCAVKGFYPSDIAVEWESNGQPENNYKTTP | |
| | PVLDSDGSFFLVSKLTVDKSRWQQGNVFSCSVMHEA | |
| | LHNHYTQKSLSLSPGK | |
| NKG2D-IL2v L19V alias NKG2D- | EVQLLESGGGLVQPGGSLRLSCAASGFTFSSYAMSW | 53 |
| IL2QM-F-09-07 (P1AE2765) - HC | VRQAPGKGLEWVSAISGSGGSTYYADSVKGRFTISR | |
| knob | DNSKNTLYLQMNSLRAEDTAVYYCAKELYREYMDYW | |
| | GQGTLVTVSSASTKGPSVFPLAPSSKSTSGGTAALG | |
| | CLVKDYFPEPVTVSWNSGALTSGVHTFPAVLQSSGL | |
| | YSLSSVVTVPSSSLGTQTYICNVNHKPSNTKVDKKV | |
| | EPKSCDKTHTCPPCPAPEAAGGPSVFLFPPKPKDTL | |
| | MISRTPEVTCVVVDVSHEDPEVKFNWYVDGVEVHNA | |
| | KTKPREEQYNSTYRVVSVLTVLHQDWLNGKEYKCKV | |
| | SNKALGAPIEKTISKAKGQPREPQVYTLPPCRDELT | |
| | KNQVSLWCLVKGFYPSDIAVEWESNGQPENNYKTTP | |
| | PVLDSDGSFFLYSKLTVDKSRWQQGNVFSCSVMHEA | |
| | LHNHYTQKSLSLSPGGGGGSGGGGSGGGGSAPASSS | |
| | TKKTQLQLEHLLVDLQMILNGINNYKNPKLTRMLTA | |
| | KFAMPKKATELKHLOCLEEELKPLEEVLNGAQSKNF | |
| | HLRPRDLISNINVIVLELKGSETTFMCEYADETATI | |
| | VEFLNRWITFAQSIISTLT | |
| NKG2D-IL2v L80A alias NKG2D- | SSELTQDPAVSVALGQTVRITCQGDSLRSYYASWYQ | 49 |
| IL2QM-F-09-08 (P1AE2766) - LC | QKPGQAPVLVIYGKNNRPSGIPDRFSGSSSGNTASL | |
| | TITGAQAEDEADYYCNSRDSFSIHQNVFGGGTKLTV | |
| | LGQPKAAPSVTLFPPSSEELQANKATLVCLISDFYP | |
| | GAVTVAWKADSSPVKAGVETTTPSKQSNNKYAASSY | |
| | LSLTPEQWKSHRSYSCQVTHEGSTVEKTVAPTECS | |
| NKG2D-IL2v L80A alias NKG2D- | EVQLLESGGGLVQPGGSLRLSCAASGFTFSSYAMSW | 50 |
| IL2QM-F-09-08 (P1AE2766) - HC | VRQAPGKGLEWVSAISGSGGSTYYADSVKGRFTISR | |
| hole | DNSKNTLYLQMNSLRAEDTAVYYCAKELYREYMDYW | |
| | GQGTLVTVSSASTKGPSVFPLAPSSKSTSGGTAALG | |
| | CLVKDYFPEPVTVSWNSGALTSGVHTFPAVLQSSGL | |
| | YSLSSVVTVPSSSLGTQTYICNVNHKPSNTKVDKKV | |
| | EPKSCDKTHTCPPCPAPEAAGGPSVFLFPPKPKDTL | |
| | MISRTPEVTCVVVDVSHEDPEVKFNWYVDGVEVHNA | |
| | KTKPREEQYNSTYRVVSVLTVLHQDWLNGKEYKCKV | |
| | SNKALGAPIEKTISKAKGQPREPQVCTLPPSRDELT | |
| | KNQVSLSCAVKGFYPSDIAVEWESNGQPENNYKTTP | |
| | PVLDSDGSFFLVSKLTVDKSRWQQGNVFSCSVMHEA | |
| | LHNHYTQKSLSLSPGK | |
| NKG2D-IL2v L80A alias NKG2D- | EVQLLESGGGLVQPGGSLRLSCAASGFTFSSYAMSW | 54 |
| IL2QM-F-09-08 (P1AE2766) - HC | VRQAPGKGLEWVSAISGSGGSTYYADSVKGRFTISR | |
| knob | DNSKNTLYLQMNSLRAEDTAVYYCAKELYREYMDYW | |
| | GQGTLVTVSSASTKGPSVFPLAPSSKSTSGGTAALG | |
| | CLVKDYFPEPVTVSWNSGALTSGVHTFPAVLQSSGL | |
| | YSLSSVVTVPSSSLGTQTYICNVNHKPSNTKVDKKV | |
| | EPKSCDKTHTCPPCPAPEAAGGPSVFLFPPKPKDTL | |
| | MISRTPEVTCVVVDVSHEDPEVKFNWYVDGVEVHNA | |
| | KTKPREEQYNSTYRVVSVLTVLHQDWLNGKEYKCKV | |
| | SNKALGAPIEKTISKAKGQPREPQVYTLPPCRDELT | |
| | KNQVSLWCLVKGFYPSDIAVEWESNGQPENNYKTTP | |
| | PVLDSDGSFFLYSKLTVDKSRWQQGNVFSCSVMHEA | |
| | LHNHYTQKSLSLSPGGGGGSGGGGSGGGGSAPASSS | |
| | TKKTQLQLEHLLLDLQMILNGINNYKNPKLTRMLTA | |
| | KFAMPKKATELKHLQCLEEELKPLEEVLNGAQSKNF | |
| | HARPRDLISNINVIVLELKGSETTFMCEYADETATI | |
| | VEFLNRWITFAQSIISTLT | |
| NKG2D-IL2v Q126T alias | DIQMTQSPSSLSASVGDRVTITCRASQDISGALNWY | 55 |
| NKG2D 80 IgG-huIL2v Q126T | QQKPGKAPNLLIYAASSLQSGVPSRFSGSGSGTDYT | |
| (P1AF2141) - LC | LTISSLQPEDFATYYCQQANSFPLTFGGGTKVEIKR | |
| | TVAAPSVFIFPPSDEQLKSGTASVVCLLNNFYPREA | |
| | KVQWKVDNALQSGNSQESVTEQDSKDSTYSLSSTLT | |
| | LSKADYEKHKVYACEVTHQGLSSPVTKSFNRGEC | |
| NKG2D-IL2v Q126T alias | QEQLVQSGAEVKKPGESLKISCKGSGFDFNTFWMTW | 56 |
| NKG2D 80 IgG-huIL2v Q126T | VRQMPGKGLEWMGSIHGGSGSRDYSPSFQGQVTISA | |
| (P1AF2141) - HC hole | DIDQSTAYLOWSSLKASDTAMYYCARPGYRSWSKTF | |
| | DLWGQGTTVTVSSASTKGPSVFPLAPSSKSTSGGTA | |
| | ALGCLVKDYFPEPVTVSWNSGALTSGVHTFPAVLQS | |
| | SGLYSLSSVVTVPSSSLGTQTYICNVNHKPSNTKVD | |
| | KKVEPKSCDKTHTCPPCPAPEAAGGPSVFLFPPKPK | |
| | DTLMISRTPEVTCVVVDVSHEDPEVKFNWYVDGVEV | |
| | HNAKTKPREEQYNSTYRVVSVLTVLHQDWLNGKEYK | |
| | CKVSNKALGAPIEKTISKAKGQPREPQVCTLPPSRD | |
| | ELTKNQVSLSCAVKGFYPSDIAVEWESNGQPENNYK | |
| | TTPPVLDSDGSFFLVSKLTVDKSRWQQGNVFSCSVM | |
| | HEALHNHYTQKSLSLSPGK | |
| NKG2D-IL2v Q126T alias | QEQLVQSGAEVKKPGESLKISCKGSGFDFNTFWMTW | 57 |
| NKG2D 80 IgG-huIL2v Q126T | VRQMPGKGLEWMGSIHGGSGSRDYSPSFQGQVTISA | |
| (P1AF2141) - HC knob | DIDQSTAYLQWSSLKASDTAMYYCARPGYRSWSKTF | |
| | DLWGQGTTVTVSSASTKGPSVFPLAPSSKSTSGGTA | |
| | ALGCLVKDYFPEPVTVSWNSGALTSGVHTFPAVLQS | |
| | SGLYSLSSVVTVPSSSLGTQTYICNVNHKPSNTKVD | |
| | KKVEPKSCDKTHTCPPCPAPEAAGGPSVFLFPPKPK | |
| | DTLMISRTPEVTCVVVDVSHEDPEVKFNWYVDGVEV | |
| | HNAKTKPREEQYNSTYRVVSVLTVLHQDWLNGKEYK | |
| | CKVSNKALGAPIEKTISKAKGQPREPQVYTLPPCRD | |
| | ELTKNQVSLWCLVKGFYPSDIAVEWESNGQPENNYK | |
| | TTPPVLDSDGSFFLYSKLTVDKSRWQQGNVFSCSVM | |
| | HEALHNHYTQKSLSLSPGGGGGSGGGGSGGGGSAPA | |
| | SSSTKKTQLQLEHLLLDLQMILNGINNYKNPKLTRM | |
| | LTAKFAMPKKATELKHLQCLEEELKPLEEVLNGAQS | |
| | KNFHLRPRDLISNINVIVLELKGSETTFMCEYADET | |
| | ATIVEFLNRWITFATSIISTLT | |
| ratPD1 - mulgG1-DAPG-DD/KK - | DIVMTQGTLPNPVPSGESVSITCRSSKSLLYSDGKT | 58 |
| IL2v non-masked (P1AG9991) - | YLNWYLQRPGQSPQLLIYWMSTRASGVSDRESGSGS | |
| LC | GTDFTLKISGVEAEDVGIYYCQQGLEFPTFGGGTKL | |
| | ELKRADAAPTVSIFPPSSEQLTSGGASVVCFLNNFY | |
| | PKDINVKWKIDGSERQNGVLNSWTDQDSKDSTYSMS | |
| | STLTLTKDEYERHNSYTCEATHKTSTSPIVKSFNRN | |
| | EC | |
| ratPD1 - mulgG1-DAPG-DD/KK - | EVQLQESGPGLVKPSQSLSLTCSVTGYSITSSYRWN | 59 |
| IL2v non-masked (P1AG9991) - | WIRKFPGNRLEWMGYINSAGISNYNPSLKRRISITR | |
| HC (KK+) | DTSKNQFFLQVNSVTTEDAATYYCARSDNMGTTPFT | |
| | YWGQGTLVTVSSAKTTPPSVYPLAPGSAAQTNSMVT | |
| | LGCLVKGYFPEPVTVTWNSGSLSSGVHTFPAVLQSD | |
| | LYTLSSSVTVPSSTWPSQTVTCNVAHPASSTKVDKK | |
| | IVPRDCGCKPCICTVPEVSSVFIFPPKPKDVLTITL | |
| | TPKVTCVVVAISKDDPEVQFSWFVDDVEVHTAQTKP | |
| | REEQINSTFRSVSELPIMHQDWLNGKEFKCRVNSAA | |
| | FGAPIEKTISKTKGRPKAPQVYTIPPPKKQMAKDKV | |
| | SLTCMITNFFPEDITVEWQWNGQPAENYKNTQPIMK | |
| | TDGSYFVYSKLNVQKSNWEAGNTFTCSVLHEGLHNH | |
| | HTEKSLSHSPG | |
| ratPD1 - mulgG1-DAPG-DD/KK - | EVQLQESGPGLVKPSQSLSLTCSVTGYSITSSYRWN | 60 |
| IL2v non-masked (P1AG9991) - | WIRKFPGNRLEWMGYINSAGISNYNPSLKRRISITR | |
| HC (DD-) - IL2v | DTSKNQFFLQVNSVTTEDAATYYCARSDNMGTTPFT | |
| | YWGQGTLVTVSSAKTTPPSVYPLAPGSAAQTNSMVT | |
| | LGCLVKGYFPEPVTVTWNSGSLSSGVHTFPAVLQSD | |
| | LYTLSSSVTVPSSTWPSQTVTCNVAHPASSTKVDKK | |
| | IVPRDCGCKPCICTVPEVSSVFIFPPKPKDVLTITL | |
| | TPKVTCVVVAISKDDPEVQFSWFVDDVEVHTAQTKP | |
| | REEQINSTFRSVSELPIMHQDWLNGKEFKCRVNSAA | |
| | FGAPIEKTISKTKGRPKAPQVYTIPPPKEQMAKDKV | |
| | SLTCMITNFFPEDITVEWQWNGQPAENYDNTQPIMD | |
| | TDGSYFVYSDLNVQKSNWEAGNTFTCSVLHEGLHNH | |
| | HTEKSLSHSPGGGGGSGGGGSGGGGGAPASSSTKKT | |
| | QLQLEHLLLDLQMILNGINNYKNPKLTRMLTAKFAM | |
| | PKKATELKHLQCLEEELKPLEEVLNGAQSKNFHLRP | |
| | RDLISNINVIVLELKGSETTFMCEYADETATIVEFL | |
| | NRWITFAQSIISTLT | |
| ratPD1 mulgG1-huIL2v Q126T | DIVMTQGTLPNPVPSGESVSITCRSSKSLLYSDGKT | 61 |
| DAPG-DD/KK (P1AG8304) - LC | YLNWYLQRPGQSPQLLIYWMSTRASGVSDRFSGSGS | |
| | GTDFTLKISGVEAEDVGIYYCQQGLEFPTFGGGTKL | |
| | ELKRADAAPTVSIFPPSSEQLTSGGASVVCFLNNFY | |
| | PKDINVKWKIDGSERQNGVLNSWTDQDSKDSTYSMS | |
| | STLTLTKDEYERHNSYTCEATHKTSTSPIVKSFNRN | |
| | EC | |
| ratPD1 mulgG1-huIL2v Q126T | EVQLQESGPGLVKPSQSLSLTCSVTGYSITSSYRWN | 62 |
| DAPG-DD/KK (P1AG8304) - HC | WIRKFPGNRLEWMGYINSAGISNYNPSLKRRISITR | |
| (KK+) | DTSKNQFFLQVNSVTTEDAATYYCARSDNMGTTPFT | |
| | YWGQGTLVTVSSAKTTPPSVYPLAPGSAAQTNSMVT | |
| | LGCLVKGYFPEPVTVTWNSGSLSSGVHTFPAVLQSD | |
| | LYTLSSSVTVPSSTWPSQTVTCNVAHPASSTKVDKK | |
| | IVPRDCGCKPCICTVPEVSSVFIFPPKPKDVLTITL | |
| | TPKVTCVVVAISKDDPEVQFSWFVDDVEVHTAQTKP | |
| | REEQINSTFRSVSELPIMHQDWLNGKEFKCRVNSAA | |
| | FGAPIEKTISKTKGRPKAPQVYTIPPPKKQMAKDKV | |
| | SLTCMITNFFPEDITVEWQWNGQPAENYKNTQPIMK | |
| | TDGSYFVYSKLNVQKSNWEAGNTFTCSVLHEGLHNH | |
| | HTEKSLSHSPG | |
| ratPD1 mulgG1-huIL2v Q126T | EVQLQESGPGLVKPSQSLSLTCSVTGYSITSSYRWN | 63 |
| DAPG-DD/KK (P1AG8304) - HC | WIRKFPGNRLEWMGYINSAGISNYNPSLKRRISITR | |
| (DD-) - IL2v Q126T | DTSKNQFFLQVNSVTTEDAATYYCARSDNMGTTPFT | |
| | YWGQGTLVTVSSAKTTPPSVYPLAPGSAAQTNSMVT | |
| | LGCLVKGYFPEPVTVTWNSGSLSSGVHTFPAVLQSD | |
| | LYTLSSSVTVPSSTWPSQTVTCNVAHPASSTKVDKK | |
| | IVPRDCGCKPCICTVPEVSSVFIFPPKPKDVLTITL | |
| | TPKVTCVVVAISKDDPEVQFSWFVDDVEVHTAQTKP | |
| | REEQINSTFRSVSELPIMHQDWLNGKEFKCRVNSAA | |
| | FGAPIEKTISKTKGRPKAPQVYTIPPPKEQMAKDKV | |
| | SLTCMITNFFPEDITVEWQWNGQPAENYDNTQPIMD | |
| | TDGSYFVYSDLNVQKSNWEAGNTFTCSVLHEGLHNH | |
| | HTEKSLSHSPGGGGGSGGGGSGGGGGAPASSSTKKT | |
| | QLQLEHLLLDLQMILNGINNYKNPKLTRMLTAKFAM | |
| | PKKATELKHLQCLEEELKPLEEVLNGAQSKNFHLRP | |
| | RDLISNINVIVLELKGSETTFMCEYADETATIVEFL | |
| | NRWITFATSIISTLT | |
| PD1-LAG3-IL2v (P1AF4801) - | EVQLLESGGGLVQPGGSLRLSCAASGFSFSSYTMSW | 64 |
| LC1 (PD1, crossed) | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASVAAPSVFIFPPSDEQLKSGTAS | |
| | VVCLLNNFYPREAKVQWKVDNALQSGNSQESVTEQD | |
| | SKDSTYSLSSTLTLSKADYEKHKVYACEVTHQGLSS | |
| | PVTKSFNRGEC | |
| PD1-LAG3-IL2v (P1AF4801) - | DIQMTQSPSSLSASVGDRVTITCRASQSISSYLNWY | 65 |
| LC2 (LAG3, charges) | QQKPGKAPKLLIYAASTLQSGVPSRFSGSGSGTDFT | |
| | LTISSLQPEDFATYYCQQTYSSPLTFGGGTKVEIKR | |
| | TVAAPSVFIFPPSDRKLKSGTASVVCLLNNFYPREA | |
| | KVQWKVDNALQSGNSQESVTEQDSKDSTYSLSSTLT | |
| | LSKADYEKHKVYACEVTHQGLSSPVTKSFNRGEC | |
| PD1-LAG3-IL2v (P1AF4801) - | EVQLLESGGGLVQPGGSLRLSCAASGFIFDDYTMNW | 66 |
| HC hole (LAG3, charges) | VRQAPGKGLEWVAVISWDGGGTYYTDSVKGRFTISR | |
| | DDFKNTLYLQMNSLRAEDTAVYYCAKGLTDTTLYGS | |
| | DYWGQGTLVTVSSASTKGPSVFPLAPSSKSTSGGTA | |
| | ALGCLVEDYFPEPVTVSWNSGALTSGVHTFPAVLQS | |
| | SGLYSLSSVVTVPSSSLGTQTYICNVNHKPSNTKVD | |
| | EKVEPKSCDKTHTCPPCPAPEAAGGPSVFLFPPKPK | |
| | DTLMISRTPEVTCVVVDVSHEDPEVKFNWYVDGVEV | |
| | HNAKTKPREEQYNSTYRVVSVLTVLHQDWLNGKEYK | |
| | CKVSNKALGAPIEKTISKAKGQPREPQVCTLPPSRD | |
| | ELTKNQVSLSCAVKGFYPSDIAVEWESNGQPENNYK | |
| | TTPPVLDSDGSFFLVSKLTVDKSRWQQGNVFSCSVM | |
| | HEALHNHYTQKSLSLSPGK | |
| PD1-LAG3-IL2v (P1AF4801) - | DIVMTQSPDSLAVSLGERATINCKASESVDTSDNSF | 67 |
| HC knob (PD1, crossed) - IL2v | IHWYQQKPGQSPKLLIYRSSTLESGVPDRFSGSGSG | |
| | TDFTLTISSLQAEDVAVYYCQQNYDVPWTFGQGTKV | |
| | EIKSSASTKGPSVFPLAPSSKSTSGGTAALGCLVKD | |
| | YFPEPVTVSWNSGALTSGVHTFPAVLQSSGLYSLSS | |
| | VVTVPSSSLGTQTYICNVNHKPSNTKVDKKVEPKSC | |
| | DKTHTCPPCPAPEAAGGPSVFLFPPKPKDTLMISRT | |
| | PEVTCVVVDVSHEDPEVKFNWYVDGVEVHNAKTKPR | |
| | EEQYNSTYRVVSVLTVLHQDWLNGKEYKCKVSNKAL | |
| | GAPIEKTISKAKGQPREPQVYTLPPCRDELTKNQVS | |
| | LWCLVKGFYPSDIAVEWESNGQPENNYKTTPPVLDS | |
| | DGSFFLYSKLTVDKSRWQQGNVFSCSVMHEALHNHY | |
| | TQKSLSLSPGGGGGSGGGGSGGGGSAPASSSTKKTQ | |
| | LQLEHLLLDLQMILNGINNYKNPKLTRMLTAKFAMP | |
| | KKATELKHLQCLEEELKPLEEVLNGAQSKNFHLRPR | |
| | DLISNINVIVLELKGSETTFMCEYADETATIVEFLN | |
| | RWITFAQSIISTLT | |
| PD1-LAG3-IL2v Q126T | EVQLLESGGGLVQPGGSLRLSCAASGFSFSSYTMSW | 68 |
| (P1AF7951) - LC1 (PD1, crossed) | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASVAAPSVFIFPPSDEQLKSGTAS | |
| | VVCLLNNFYPREAKVQWKVDNALQSGNSQESVTEQD | |
| | SKDSTYSLSSTLTLSKADYEKHKVYACEVTHQGLSS | |
| | PVTKSFNRGEC | |
| PD1-LAG3-IL2v Q126T | DIQMTQSPSSLSASVGDRVTITCRASQSISSYLNWY | 69 |
| (P1AF7951) - LC2 (LAG3, | QQKPGKAPKLLIYAASTLQSGVPSRFSGSGSGTDFT | |
| charges) | LTISSLOPEDFATYYCQQTYSSPLTFGGGTKVEIKR | |
| | TVAAPSVFIFPPSDRKLKSGTASVVCLLNNFYPREA | |
| | KVQWKVDNALQSGNSQESVTEQDSKDSTYSLSSTLT | |
| | LSKADYEKHKVYACEVTHQGLSSPVTKSFNR | |
| | GEC | |
| PD1-LAG3-IL2v Q126T | EVQLLESGGGLVQPGGSLRLSCAASGFIFDDYTMNW | 70 |
| (P1AF7951) - HC hole (LAG3, | VRQAPGKGLEWVAVISWDGGGTYYTDSVKGRFTISR | |
| charges) | DDFKNTLYLQMNSLRAEDTAVYYCAKGLTDTTLYGS | |
| | DYWGQGTLVTVSSASTKGPSVFPLAPSSKSTSGGTA | |
| | ALGCLVEDYFPEPVTVSWNSGALTSGVHTFPAVLQS | |
| | SGLYSLSSVVTVPSSSLGTQTYICNVNHKPSNTKVD | |
| | EKVEPKSCDKTHTCPPCPAPEAAGGPSVFLFPPKPK | |
| | DTLMISRTPEVTCVVVDVSHEDPEVKFNWYVDGVEV | |
| | HNAKTKPREEQYNSTYRVVSVLTVLHQDWLNGKEYK | |
| | CKVSNKALGAPIEKTISKAKGQPREPQVCTLPPSRD | |
| | ELTKNQVSLSCAVKGFYPSDIAVEWESNGQPENNYK | |
| | TTPPVLDSDGSFFLVSKLTVDKSRWQQGNVFSCSVM | |
| | HEALHNHYTQKSLSLSPGK | |
| PD1-LAG3-IL2v Q126T | DIVMTQSPDSLAVSLGERATINCKASESVDTSDNSF | 71 |
| (P1AF7951) - HC knob (PD1, | IHWYQQKPGQSPKLLIYRSSTLESGVPDRFSGSGSG | |
| crossed) - IL2v Q126T | TDFTLTISSLQAEDVAVYYCQQNYDVPWTFGQGTKV | |
| | EIKSSASTKGPSVFPLAPSSKSTSGGTAALGCLVKD | |
| | YFPEPVTVSWNSGALTSGVHTFPAVLQSSGLYSLSS | |
| | VVTVPSSSLGTQTYICNVNHKPSNTKVDKKVEPKSC | |
| | DKTHTCPPCPAPEAAGGPSVFLFPPKPKDTLMISRT | |
| | PEVTCVVVDVSHEDPEVKFNWYVDGVEVHNAKTKPR | |
| | EEQYNSTYRVVSVLTVLHQDWLNGKEYKCKVSNKAL | |
| | GAPIEKTISKAKGQPREPQVYTLPPCRDELTKNQVS | |
| | LWCLVKGFYPSDIAVEWESNGQPENNYKTTPPVLDS | |
| | DGSFFLYSKLTVDKSRWQQGNVFSCSVMHEALHNHY | |
| | TQKSLSLSPGGGGGSGGGGSGGGGSAPASSSTKKTQ | |
| | LQLEHLLLDLQMILNGINNYKNPKLTRMLTAKFAMP | |
| | KKATELKHLQCLEEELKPLEEVLNGAQSKNFHLRPR | |
| | DLISNINVIVLELKGSETTFMCEYADETATIVEFLN | |
| | RWITFATSIISTLT | |
| anti-PD1 IgG PG LALA | DIVMTQSPDSLAVSLGERATINCKASESVDTSDNSF | 72 |
| (P1AA6888) - LC | IHWYQQKPGQSPKLLIYRSSTLESGVPDRFSGSGSG | |
| | TDFTLTISSLQAEDVAVYYCQQNYDVPWTFGQGTKV | |
| | EIKRTVAAPSVFIFPPSDEQLKSGTASVVCLLNNFY | |
| | PREAKVQWKVDNALQSGNSQESVTEQDSKDSTYSLS | |
| | STLTLSKADYEKHKVYACEVTHQGLSSPVTKSFNRG | |
| | EC | |
| anti-PD1 IgG PG LALA | EVQLLESGGGLVQPGGSLRLSCAASGFSFSSYTMSW | 73 |
| (P1AA6888) - HC | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVYTLPPSRDE | |
| | LTKNQVSLTCLVKGFYPSDIAVEWESNGQPENNYKT | |
| | TPPVLDSDGSFFLYSKLTVDKSRWQQGNVFSCSVMH | |
| | EALHNHYTQKSLSLSPGK | |
| CDRH1 PD1 Binder | SYTMS | 74 |
| CDRH2 PD1 Binder | TISGGGRDIYYPDSVKG | 75 |
| CDRH3 PD1 Binder | LTGRVYFALDS | 76 |
| CDRL1 PD1 Binder | KASESVDTSDNSFIH | 77 |
| CDRL2 PD1 Binder | RSSTLES | 78 |
| CDRL3 PD1 Binder | QQNYDVPWT | 79 |
| VH PD1 Binder | EVQLLESGGGLVQPGGSLRLSCAASGFSFSSYTMS | 80 |
| | WVRQAPGKGLEWVATISGGGRDIYYPDSVKGRFTIS | |
| | RDNSKNTLYLQMNSLRAEDTAVYYCVLLTGRVYFAL | |
| | DSWGQGTLVTVSS | |
| VL PD1 Binder | DIVMTQSPDSLAVSLGERATINCKASESVDTSDNSF | 81 |
| | IHWYQQKPGQSPKLLIYRSSTLESGVPDRFSGSGSG | |
| | TDFTLTISSLQAEDVAVYYCQQNYDVPWTFGQGTKV | |
| | EIK | |
| CDRH1 LAG3 Binder | DYTMN | 82 |
| CDRH2 LAG3 Binder | VISWDGGGTYYTDSVKG | 83 |
| CDRH3 LAG3 Binder | GLTDTTLYGSDY | 84 |
| CDRL1 LAG3 Binder | RASQSISSYLN | 85 |
| CDRL2 LAG3 Binder | AASTLOS | 86 |
| CDRL3 LAG3 Binder | QQTYSSPLT | 87 |
| VH LAG3 Binder | EVQLLESGGGLVQPGGSLRLSCAASGFIFDDYTMNW | 88 |
| | VRQAPGKGLEWVAVISWDGGGTYYTDSVKGRFTISR | |
| | DDFKNTLYLQMNSLRAEDTAVYYCAKGLTDTTLYGS | |
| | DYWGQGTLVTVSS | |
| VL LAG3 Binder | DIQMTQSPSSLSASVGDRVTITCRASQSISSYLNWY | 89 |
| | QQKPGKAPKLLIYAASTLQSGVPSRFSGSGSGTDFT | |
| | LTISSLOPEDFATYYCQQTYSSPLTFGGGTKVEIK | |
| human IL2 | APTSSSTKKTQLQLEHLLLDLQMILNGINNYKNPKL | 90 |
| | TRMLTFKFYMPKKATELKHLQCLEEELKPLEEVLNL | |
| | AQSKNFHLRPRDLISNINVIVLELKGSETTFMCEYA | |
| | DETATIVEFLNRWITFCQSIISTLT | |
| human IL2v (T3A, F42A, Y45A, | APASSSTKKTQLQLEHLLLDLQMILNGINNYKNPKL | 91 |
| L72G, C125A) | TRMLTAKFAMPKKATELKHLQCLEEELKPLEEVLNG | |
| | AQSKNFHLRPRDLISNINVIVLELKGSETTFMCEYA | |
| | DETATIVEFLNRWITFAQSIISTLT | |
| IL2v Q126T (T3A, F42A, Y45A, | APASSSTKKTQLQLEHLLLDLQMILNGINNYKNPKL | 92 |
| L72G, C125A, Q126T) | TRMLTAKFAMPKKATELKHLQCLEEELKPLEEVLNG | |
| | AQSKNFHLRPRDLISNINVIVLELKGSETTFMCEYA | |
| | DETATIVEFLNRWITFATSIISTLT | |
| linker | GGGGSGGGGSGGGGS | 93 |
| hIL-2 signal peptide | MYRMQLLSCIALSLALVTNS | 94 |
| Human IL-2 (C125A) | APTSSSTKKTQLQLEHLLLDLQMILNGINNYKNPKL | 95 |
| | TRMLTFKFYMPKKATELKHLQCLEEELKPLEEVLNL | |
| | AQSKNFHLRPRDLISNINVIVLELKGSETTFMCEYA | |
| | DETATIVEFLNRWITFAQSIISTLT | |
| hPD-1 | PGWFLDSPDRPWNPPTFSPALLVVTEGDNATFTCSF | 96 |
| (without signal | SNTSESFVLNWYRMSPSNQTDKLAAFPEDRSQPGQD | |
| sequence) | CRFRVTQLPNGRDFHMSVVRARRNDSGTYLCGAISL | |
| | APKAQIKESLRAELRVTERRAEVPTAHPSPSPRPAG | |
| | QFQTLVVGVVGGLLGSLVLLVWVLAVICSRAARGTI | |
| | GARRTGQPLKEDPSAVPVFSVDYGELDFQWREKTPE | |
| | PPVPCVPEQTEYATIVFPSGMGTSSPARRGSADGPR | |
| | SAQPLRPEDGHCSWPL | |
| hPD-1 (with | MQIPQAPWPVVWAVLQLGWRPGWFLDSPDRPWNPPT | 97 |
| signal | FSPALLVVTEGDNATFTCSFSNTSESFVLNWYRMSP | |
| sequence) | SNQTDKLAAFPEDRSQPGQDCRFRVTQLPNGRDFHM | |
| | SVVRARRNDSGTYLCGAISLAPKAQIKESLRAELRV | |
| | TERRAEVPTAHPSPSPRPAGQFQTLVVGVVGGLLGS | |
| | LVLLVWVLAVICSRAARGTIGARRTGQPLKEDPSAV | |
| | PVFSVDYGELDFQWREKTPEPPVPCVPEQTEYATIV | |
| | FPSGMGTSSPARRGSADGPRSAQPLRPEDGHCSWPL | |
| IL2v N88D (T3A, F42A, Y45A, | APASSSTKKTQLQLEHLLLDLQMILNGINNYKNPKL | 98 |
| L72G, N88D, C125A) | TRMLTAKFAMPKKATELKHLQCLEEELKPLEEVLNG | |
| | AQSKNFHLRPRDLISDINVIVLELKGSETTFMCEYA | |
| | DETATIVEFLNRWITFAQSIISTLT | |
| IL2v N88Q (T3A, F42A, Y45A, | APASSSTKKTQLQLEHLLLDLQMILNGINNYKNPKL | 99 |
| L72G, N88Q, C125A) | TRMLTAKFAMPKKATELKHLQCLEEELKPLEEVLNG | |
| | AQSKNFHLRPRDLISQINVIVLELKGSETTFMCEYA | |
| | DETATIVEFLNRWITFAQSIISTLT | |
| PD1-IL2v_N88D (P1AA6761 = | DIVMTQSPDSLAVSLGERATINCKASESVDTSDNSF | 21 |
| P1AD5401) LC | IHWYQQKPGQSPKLLIYRSSTLESGVPDRFSGSGSG | |
| | TDFTLTISSLQAEDVAVYYCQQNYDVPWTFGQGTKV | |
| | EIKRTVAAPSVFIFPPSDEQLKSGTASVVCLLNNFY | |
| | PREAKVQWKVDNALQSGNSQESVTEQDSKDSTYSLS | |
| | STLTLSKADYEKHKVYACEVTHQGLSSPVTKSFNRG | |
| | EC | |
| PD1-IL2v_N88D (P1AA6761 = | EVQLLESGGGLVQPGGSLRLSCAASGFSFSSYTMSW | 22 |
| P1AD5401) HC hole | VRQAPGKGLEWVATISGGGRDIYYPDSVKGRFTISR | |
| | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVCTLPPSRDE | |
| | LTKNQVSLSCAVKGFYPSDIAVEWESNGQPENNYKT | |
| | TPPVLDSDGSFFLVSKLTVDKSRWQQGNVFSCSVMH | |
| | EALHNRFTQKSLSLSPGK | |
| PD1-IL2v_N88D (P1AA6761 = | EVQLLESGGGLVQPGGSLRLSCAASGFSFSSYTMSW | 100 |
| P1AD5401) HC knob with IL2v | VRQAPGKGLEWVATISGGGRDIYYPDSVKGRFTISR | |
| N88D | DNSKNTLYLQMNSLRAEDTAVYYCVLLTGRVYFALD | |
| | SWGQGTLVTVSSASTKGPSVFPLAPSSKSTSGGTAA | |
| | LGCLVKDYFPEPVTVSWNSGALTSGVHTFPAVLQSS | |
| | GLYSLSSVVTVPSSSLGTQTYICNVNHKPSNTKVDK | |
| | KVEPKSCDKTHTCPPCPAPEAAGGPSVFLFPPKPKD | |
| | TLMISRTPEVTCVVVDVSHEDPEVKFNWYVDGVEVH | |
| | NAKTKPREEQYNSTYRVVSVLTVLHQDWLNGKEYKC | |
| | KVSNKALGAPIEKTISKAKGQPREPQVYTLPPCRDE | |
| | LTKNQVSLWCLVKGFYPSDIAVEWESNGQPENNYKT | |
| | TPPVLDSDGSFFLYSKLTVDKSRWQQGNVFSCSVMH | |
| | EALHNHYTQKSLSLSPGGGGGSGGGGSGGGGSAPAS | |
| | SSTKKTQLQLEHLLLDLQMILNGINNYKNPKLTRML | |
| | TAKFAMPKKATELKHLQCLEEELKPLEEVLNGAQSK | |
| | NFHLRPRDLISDINVIVLELKGSETTFMCEYADETA | |
| | TIVEFLNRWITFAQSIISTLT | |

### EXAMPLES

The following are examples of methods and compositions of the invention. It is understood that various other embodiments may be practiced, given the general description provided above.

### Example 1

### Example 1A. Molecules

The molecules tested in the following Examples consist of the amino acid sequences according

TABLE A
Molecules produced and tested in the Examples.

| Molecule | SEQ IDs |
|---|---|
| FAP-targeted IL-2(Knob/hole) (P1AA5355) | 1, 2, 3 |
| huPD1-IL2v | 4, 5, 6 |
| FAP 4B9 IgG-huIL2v (IL2qm) (P1AE4798) | 1, 2, 7 |
| FAP 4B9 IgG-huIL2v_D20T_Selectikine (P1AE4799) | 1, 2, 8 |
| FAP 4B9 IgG-huIL2_IL2Ra (P1AE4802) | 1, 2, 9 |
| FAP 4B9 IgG-IL2Ra_IL2 (P1AE4803) | 1, 2, 10 |
| FAP 4B9 IgG-huIL2v_Superkine (P1AE4815) | 1, 2, 11 |
| PD1 0379 IgG-IL2v (P1AD4370) | 12, 13, 14 |
| NKG2D-IL2v alias NKG2D-IL2QM-F-11-01 | 15, 16, 17 |
| (P1AE2767) | |
| PD1TIM3-0591-0002 (P1AA6761, P1AD5401) | 18, 19, 20 |
| PD1 376 IgG-huIL2v K8S (P1AE0488) | 21, 22, 23 |
| PD1 376 IgG-huIL2v L12V (P1AE0489) | 21, 22, 24 |
| PD1 376 IgG-huIL2v L12A (P1AE0490) | 21, 22, 25 |
| PD1 376 IgG-huIL2v L19V (P1AE0491) | 21, 22, 26 |
| PD1 376 IgG-huIL2v T51P (P1AE0492) | 21, 22, 27 |
| PD1 376 IgG-huIL2v H79S (P1AE0493) | 21, 22, 28 |
| PD1 376 IgG-huIL2v L80A (P1AE0494) | 21, 22, 29 |
| PD1 376 IgG-huIL2v N88A (P1AE0495) | 21, 22, 30 |
| PD1 376 IgG-huIL2v N88Q (P1AE0496) | 21, 22, 31 |
| PD1-IL2v_N88D (P1AA6761 = P1AD5401) | 21, 22, 100 |
| PD1 376 IgG-huIL2v D109A (P1AE0497) | 21, 22, 32 |
| PD1 376 IgG-huIL2v Q126N (P1AE0498) | 21, 22, 33 |
| PD1 376 IgG-huIL2v Q126E (P1AE0499) | 21, 22, 34 |
| PD1 376 IgG-huIL2v Q126T (P1AF2142) | 21, 22, 35 |
| FAP 4B9 IgG-huIL2v_E15V (P1AE4800) | 1, 2, 36 |
| FAP 4B9 IgG-huIL2v_E95A (P1AE4801) | 1, 2, 37 |
| FAP 4B9 IgG-huIL2v_L12A (P1AE4804) | 1, 2, 38 |
| FAP 4B9 IgG-huIL2v_L12A_L19A (P1AE4806) | 1, 2, 39 |
| FAP 4B9 IgG-huIL2v_L12A_L80A (P1AE4807) | 1, 2, 40 |
| FAP 4B9 IgG-huIL2v_L19V (P1AE4808) | 1, 2, 41 |
| FAP 4B9 IgG-huIL2v_N88T (P1AE4809) | 1, 2, 42 |
| FAP 4B9 IgG-huIL2v_N119K (P1AE4810) | 1, 2, 43 |
| FAP 4B9 IgG-huIL2v_Q22A (P1AE4811) | 1, 2, 44 |
| FAP 4B9 IgG-huIL2v_Q126T (P1AE4812) | 1, 2, 45 |
| FAP 4B9 IgG-huIL2v_S87A (P1AE4813) | 1, 2, 46 |
| FAP 4B9 IgG-huIL2v_S130A (P1AE4814) | 1, 2, 47 |
| FAP 4B9 IgG-huIL2v_T133K (P1AE4816) | 1, 2, 48 |
| NKG2D-IL2v K8S alias NKG2D-IL2QM-F-09-05 | 49, 50, 51 |
| (P1AE2763) | |
| NKG2D-IL2v L12A alias NKG2D-IL2QM-F-09-06 | 49, 50, 52 |
| (P1AE2764 | |
| NKG2D-IL2v L19V alias NKG2D-IL2QM-F-09-07 | 49, 50, 53 |
| (P1AE2765) | |
| NKG2D-IL2v L80A alias NKG2D-IL2QM-F-09-08 | 49, 50, 54 |
| (P1AE2766) | |
| NKG2D-IL2v Q126T alias NKG2D 80 IgG-huIL2v | 55, 56, 57 |
| Q126T (P1AF2141) | |
| ratPD1 - muIgG1-DAPG-DD/KK - IL2v non- | 58, 59, 60 |
| masked (P1AG9991) | |
| ratPD1 muIgG1-huIL2v Q126T DAPG-DD/KK | 61, 62, 63 |
| (P1AG8304) | |
| PD1-LAG3-IL2v (P1AF4801) | 64, 65, 66, 67 |
| PD1-LAG3-IL2v Q126T (P1AF7951) | 68, 69, 70, 71 |
| anti-PD1 IgG PG LALA (P1AA6888) | 72, 73 |

### Example 1B. Production and Analytics of Human PD1 and FAP IgG-IL2v Variants

The antibody IL2v variants fusion constructs described herein were produced in HEK cells. In some instance (surface plasmon resonance measurements) the supernatants were used directly without prior purification (Table 1). In all other assays, the proteins were first purified by ProteinA affinity chromatography and size exclusion chromatography. The end product analytics consists of monomer content determination (by analytical size exclusion chromatography) and percentage of main peak (determined by non-reduced capillary SDS electrophoresis: CE-SDS).

### Production of IgG-Like Proteins in HEK293 EBNA Cells

The antibody IL2v variants fusion constructs were generated by transient transfection of HEK293 EBNA cells. Cells were centrifuged and, medium was replaced by pre-warmed CD CHO medium (Thermo Fisher, Cat No 10743029). Expression vectors were mixed in CD CHO medium, PEI (Polyethylenimine, Polysciences, Inc, Cat No 23966-1) was added, the solution vortexed and incubated for 10 minutes at room temperature. Afterwards, cells (2 Mio/ml) were mixed with the vector/PEI solution, transferred to a flask and incubated for 3 hours at 37° C. in a shaking incubator with a 5% CO₂ atmosphere. After the incubation, Excell medium with supplements (80% of total volume) was added (W. Zhou and A. Kantardjieff, Mammalian Cell Cultures for Biologics Manufacturing, DOI: 10.1007/978-3-642-54050-9; 2014). One day after transfection, supplements (Feed, 12% of total volume) were added. Cell supernatants were harvested after 7 days by centrifugation and subsequent filtration (0.2 μm filter), and proteins were purified from the harvested supernatant by standard methods as indicated below.

### Titer Determination (PA-HPLC)

Quantification of Fc containing constructs in supernatants was performed by Protein A—HPLC on an Agilent HPLC System with UV detector. Supernatants are injected on POROS 20 A (Applied Biosystems), washed with 10 mM Tris, 50 mM Glycine, 100 mM NaCl, pH 8.0 and eluted in the same buffer at pH 2.0. The eluted peak area at 280 nm is integrated and converted to concentration by use of a calibration curve with standards analyzed in the same run.

### Purification of IgG-Like Proteins

Proteins were purified from filtered cell culture supernatants referring to standard protocols. In brief, Fc containing proteins were purified from cell culture supernatants by Protein A-affinity chromatography (equilibration buffer: 20 mM sodium citrate, 20 mM sodium phosphate, pH 7.5; elution buffer: 20 mM sodium citrate, pH 3.0). Elution was achieved at pH 3.0 followed by immediate pH neutralization of the sample. The protein was concentrated by centrifugation (Millipore Amicon® ULTRA-15 (Art.Nr.: UFC903096), and aggregated protein was separated from monomeric protein by size exclusion chromatography in 20 mM histidine, 140 mM sodium chloride, pH 6.0.

### Analytics of IgG-Like Proteins

The concentrations of purified proteins were determined by measuring the absorption at 280 nm using the mass extinction coefficient calculated on the basis of the amino acid sequence according to Pace, et al., Protein Science, 1995, 4, 2411-1423. Purity and molecular weight of the proteins were analyzed by CE-SDS in the presence and absence of a reducing agent using a LabChipGXII or LabChip GX Touch (Perkin Elmer) (Perkin Elmer). Determination of the aggregate content was performed by HPLC chromatography at 25° C. using analytical size-exclusion column (TSKgel G3000 SW XL or UP-SW3000) equilibrated in running buffer (200 mM KH₂PO₄, 250 mM KCl pH 6.2, 0.02% NaN₃).

TABLE 1
Titer determination of harvested HEK supernatants
determined by ProteinA-HPLC.

| IL2v variants | TAPIR ID | Concentration (μg/ml) |
|---|---|---|
| PD1 376 IgG-huIL2v K8S | P1AE0488-001 | 66 |
| PD1 376 IgG-huIL2v L12V | P1AE0489-001 | 57 |
| PD1 376 IgG-huIL2v L12A | P1AE0490-001 | 58 |
| PD1 376 IgG-huIL2v L19V | P1AE0491-001 | 62 |
| PD1 376 IgG-huIL2v T51P | P1AE0492-001 | 50 |
| PD1 376 IgG-huIL2v H79S | P1AE0493-001 | 63 |
| PD1 376 IgG-huIL2v L80A | P1AE0494-001 | 52 |
| PD1 376 IgG-huIL2v N88A | P1AE0495-001 | 55 |
| PD1 376 IgG-huIL2v N88Q | P1AE0496-001 | 57 |
| PD1 376 IgG-huIL2v D109A | P1AE0497-001 | 50 |
| PD1 376 IgG-huIL2v Q126N | P1AE0498-001 | 25 |
| PD1 376 IgG-huIL2v Q126E | P1AE0499-001 | 24 |

TABLE 2
Monomer product peak, high molecular weight (HMW) and low molecular weight
(LMW) side products determined by analytical size exclusion chromatography.

| | | Monomer | HMW | LMW |
|---|---|---|---|---|
| IL2v variants | TAPIR ID | peak (%) | peak (%) | peak (%) |
| PD1 376 IgG-huIL2v K8S | P1AE0488-004 | 100 | 0 | 0 |
| PD1 376 IgG-huIL2v L12V | P1AE0489-007 | 98 | 0.7 | 1.3 |
| PD1 376 IgG-huIL2v L12A | P1AE0490-004 | 98.3 | 0 | 1.7 |
| PD1 376 IgG-huIL2v L19V | P1AE0491-004 | 100 | 0 | 0 |
| PD1 376 IgG-huIL2v H79S | P1AE0493-004 | 100 | 0 | 0 |
| PD1 376 IgG-huIL2v L80A | P1AE0494-004 | 100 | 0 | 0 |
| PD1 376 IgG-huIL2v N88A | P1AE0495-004 | 98 | 0.8 | 1.2 |
| PD1 376 IgG-huIL2v N88Q | P1AE0496-004 | 100 | 0 | 0 |
| PD1 376 IgG-huIL2v D109A | P1AE0497-004 | 100 | 0 | 0 |
| FAP 4B9 IgG-huIL2v_D20T | P1AE4799-003 | 87 | 1.4 | 11.6 |
| FAP 4B9 IgG-huIL2v_Q126T | P1AE4812-003 | 100 | 0 | 0 |
| FAP 4B9 IgG-huIL2v_L12A_L80A | P1AE4807-003 | 91.3 | 0 | 8.7 |
| FAP 4B9 IgG-huIL2v_L12A_L19A | P1AE4806-003 | 93.4 | 0 | 6.7 |
| FAP 4B9 IgG-huIL2v_L12A | P1AE4804-003 | 88.7 | 0 | 11.3 |
| FAP 4B9 IgG-huIL2v_L19V | P1AE4808-003 | 91.3 | 0 | 8.7 |
| FAP 4B9 IgG-huIL2v_E15V | P1AE4800-003 | 88.9 | 0 | 11.1 |
| FAP 4B9 IgG-huIL2v_S87A | P1AE4813-003 | 93 | 0 | 7 |
| FAP 4B9 IgG-huIL2v_N88T | P1AE4809-003 | 96.1 | 0 | 3.9 |
| FAP 4B9 IgG-huIL2v_E95A | P1AE4801-003 | 92.8 | 0 | 7.2 |
| FAP 4B9 IgG-huIL2v_Q22A | P1AE4811-003 | 57.8 | 0.9 | 48.8 |
| FAP 4B9 IgG-huIL2v_N119K | P1AE4810-003 | 93.3 | 0.7 | 6 |
| FAP 4B9 IgG-huIL2v_S130A | P1AE4814-003 | 91.6 | 0 | 8.4 |
| FAP 4B9 IgG-huIL2v_T133K | P1AE4816-003 | 95.9 | 1.5 | 2.6 |

TABLE 3
Main product peak determined by non-reduced CE-SDS.

| | | Main |
|---|---|---|
| IL2v variants | TAPIR ID | peak (%) |
| PD1 376 IgG-huIL2v K8S | P1AE0488-004 | 99 |
| PD1 376 IgG-huIL2v L12V | P1AE0489-007 | 91 |
| PD1 376 IgG-huIL2v L12A | P1AE0490-004 | 95 |
| PD1 376 IgG-huIL2v L19V | P1AE0491-004 | 99 |
| PD1 376 IgG-huIL2v H79S | P1AE0493-004 | 100 |
| (N-glycosylation site in IL2v | | (65 + 35) |
| variant leads to double peak) | | |
| PD1 376 IgG-huIL2v L80A | P1AE0494-004 | 99 |
| PD1 376 IgG-huIL2v N88A | P1AE0495-004 | 88 |
| PD1 376 IgG-huIL2v N88Q | P1AE0496-004 | 89 |
| PD1 376 IgG-huIL2v D109A | P1AE0497-004 | 99 |
| FAP 4B9 IgG-huIL2v_D20T | P1AE4799-003 | 91.5 |
| FAP 4B9 IgG-huIL2v_Q126T | P1AE4812-003 | 96 |
| FAP 4B9 IgG-huIL2v_L12A_L80A | P1AE4807-003 | 100 |
| FAP 4B9 IgG-huIL2v_L12A_L19A | P1AE4806-003 | 97.1 |
| FAP 4B9 IgG-huIL2v_L12A | P1AE4804-003 | 100 |
| FAP 4B9 IgG-huIL2v_L19V | P1AE4808-003 | 100 |
| FAP 4B9 IgG-huIL2v_E15V | P1AE4800-003 | 100 |
| FAP 4B9 IgG-huIL2v_S87A | P1AE4813-003 | 96.7 |
| FAP 4B9 IgG-huIL2v_N88T | P1AE4809-003 | 100 |
| FAP 4B9 IgG-huIL2v_E95A | P1AE4801-003 | 93.1 |
| FAP 4B9 IgG-huIL2v_Q22A | P1AE4811-003 | 91.6 |
| FAP 4B9 IgG-huIL2v_N119K | P1AE4810-003 | 97.3 |
| FAP 4B9 IgG-huIL2v_S130A | P1AE4814-003 | 95.6 |
| FAP 4B9 IgG-huIL2v_T133K | P1AE4816-003 | 100 |

### Results

The IgG-IL2v variants constructs produced in HEK cells were tested either from supernatant without prior purification, but after quantification by ProteinA titer determination (Table 1) or after purification. The quality analysis of the purified material revealed that the product peak was between 57.8 (in one case) or 87% and 100% by analytical size exclusion chromatography analysis (Table 2) and between 88 and 99 by non-reduced capillary electrophoresis (Table 3).

### Conclusion

All IgG-IL2v variants were produced in good quality, except for IL2v Q22A, IL2v Q126N and IL2v Q126E.

### Example 1C. Affinity of Purified FAP-IL2v Variants to Recombinant Human IL2Rbeta-Gamma-Fc Heterodimer

### Settings

Instrumentation: Biacore T200
Chip: CM5 (#697)
Fcl to 4: anti-human Fc specific (Roche in house)
Capture: 10 nM FAP-IL2v variants s for 40 s
Analyte: human IL2R beta-gamma Fc (P1AD7029-002)
Running buffer: HBS-EP
T°: 25° C.
Dilution: 5-fold dilution in HBS-EP from 3.2 to 2000 nM
Flow: 30 μl/min
Association: 240 sec
Dissociation: 120 or 600 sec
Regeneration: 10 mM glycine pH 2.1 for 30 sec

Surface plasmon resonance (SPR) experiments were performed on a Biacore T200 with HBS-EP+ as running buffer (0.01 M HEPES pH 7.4, 0.15 M NaCl, 0.005% Surfactant P20 (BR-1006-69, Cytiva)). Anti-human Fe specific antibodies (Roche internal) were directly immobilized by amine coupling on a CM5 chip (Cytiva). The FAP-IL2v variants were captured for 40 s at 10 nM. A five-fold dilution series of the human IL2R beta-gamma Fc was passed over the ligand at 30 μl/min for 240 sec to record the association phase. The dissociation phase was monitored for 120 or 600 s and triggered by switching from the sample solution to HBS-EP+. The chip surface was regenerated after every cycle using one injection of 10 mM glycine pH 2.1 for 30 sec. Bulk refractive index differences were corrected for by subtracting the response obtained on the reference flow cell 1. The affinity constants were derived from the kinetic rate constants by fitting to a 1:1 Langmuir binding using the Biaeval software (Cytiva).

### Samples

The following samples were analyzed for binding to human IL2Rbeta-gamma-Fc (Table 4).

TABLE 4
Description of the samples analyzed for
binding to human IL2Rbeta-gamma-Fc.

| Captured molecule | TAPIR ID | Format |
|---|---|---|
| FAP 4B9 IgG-huIL2v_D20T | P1AE4799-003 | purified |
| FAP 4B9 IgG-huIL2v_Q126T | P1AE4812-003 | purified |
| FAP 4B9 IgG-huIL2v_L12A_L80A | P1AE4807-003 | purified |
| FAP 4B9 IgG-huIL2v_L12A_L19A | P1AE4806-003 | purified |
| FAP 4B9 IgG-huIL2v_L12A | P1AE4804-003 | purified |
| FAP 4B9 IgG-huIL2v_L19V | P1AE4808-003 | purified |
| FAP 4B9 IgG-huIL2v_E15V | P1AE4800-003 | purified |
| FAP 4B9 IgG-huIL2v_S87A | P1AE4813-003 | purified |
| FAP 4B9 IgG-huIL2v_N88T | P1AE4809-003 | purified |
| FAP 4B9 IgG-huIL2v_E95A | P1AE4801-003 | purified |
| FAP 4B9 IgG-huIL2v_Q22A | P1AE4811-003 | purified |
| FAP 4B9 IgG-huIL2v_N119K | P1AE4810-003 | purified |
| FAP 4B9 IgG-huIL2v_S130A | P1AE4814-003 | purified |
| FAP 4B9 IgG-huIL2v_T133K | P1AE4816-003 | purified |
| FAP 4B9 IgG-huIL2v (G4S)5 | P1AE4798-003 | purified, |
| | | reference |
| hu IL2R beta-gamma-Fc | P1AD7029-002 | Antigen used as |
| | | analyte, purified |

### Results

### Affinity Determination of Fourteen FAP-IL2v Variants to Recombinant Human IL2Rbeta-Gamma-Fc Heterodimer

The measure of the affinity to human IL-2Rbeta-gamma shows a very slow off-rate reaching the limit of the instrument and giving unrealistic KDs. However a reduced affinity can still be assessed (Table 5).

TABLE 5
Kinetic constants (1:1 Langmuir binding).

| Binder | TAPIR ID | ka (1/Ms) | kd (1/s) | KD (nM) | Comment |
|---|---|---|---|---|---|
| FAP 4B9 IgG-huIL2v_D20T | P1AE4799-003 | 2.75E+04 | 8.16E−04 | 30 | reduced affinity |
| FAP 4B9 IgG-huIL2v_Q126T | P1AE4812-003 | 1.66E+04 | 7.37E−04 | 45 | reduced affinity |
| FAP 4B9 IgG-huIL2v_L12A_L80A | P1AE4807-003 | 4.56E+04 | 3.24E−05 | 0.7 | |
| FAP 4B9 IgG-huIL2v_L12A_L19A | P1AE4806-003 | 2.72E+04 | 9.35E−05 | 3 | slightly reduced |
| | | | | | affinity |
| FAP 4B9 IgG-huIL2v_L12A | P1AE4804-003 | 3.9E+04 | 2.58E−06 | 0.06 | |
| FAP 4B9 IgG-huIL2v_L19V | P1AE4808-003 | 4.19E+04 | 6.78E−06 | 0.16 | |
| FAP 4B9 IgG-huIL2v_E15V | P1AE4800-003 | 3.94E+04 | 2.94E−06 | 0.07 | |
| FAP 4B9 IgG-huIL2v_S87A | P1AE4813-003 | 4.05E+04 | 3.02E−05 | 0.7 | |
| FAP 4B9 IgG-huIL2v_N88T | P1AE4809-003 | 2.96E+04 | 1.54E−03 | 50 | reduced affinity |
| FAP 4B9 IgG-huIL2v_E95A | P1AE4801-003 | 2.54E+04 | 2.35E−06 | 0.09 | |
| FAP 4B9 IgG-huIL2v_Q22A | P1AE4811-003 | 3.44E+04 | 9.98E−07 | 0.03 | |
| FAP 4B9 IgG-huIL2v_N119K | P1AE4810-003 | 3.24E+04 | 4.07E−07 | 0.01 | |
| FAP 4B9 IgG-huIL2v_S130A | P1AE4814-003 | 3.45E+04 | 4.23E−06 | 0.1 | |
| FAP 4B9 IgG-huIL2v_T133K | P1AE4816-003 | 3.3E+04 | 1.07E−07 | 0.003 | |
| FAP 4B9 IgG-huIL2v (G4S)5 | P1AE4798-003 | 4.12E+04 | 1.07E−06 | 0.04 | |

### Conclusion

The FAP-IL2v variants were purified and their affinity to IL2Rbeta-gamma-Fc was measured. The D20T, Q126T and N88T variants showed reduced affinity to human IL2R beta-gamma-Fe. The double-mutant L12A/L19A showed slightly reduced affinity to human IL2R beta-gamma-Fc.

### Example 1D. Characterization of PD1-IL2v Variants

Binding Assessment of PD1-IL2v Variants from Supernatants to Recombinant Human IL2Rbeta-Gamma-Fc Heterodimer

### Settings

Instrumentation: Biacore T200
Chip: CM5 (#593)
Fel to 4: anti-human Fab specific (Cytiva 28-9583-25)
Capture: Supernatants were captured to reach around 200 RU
Analyte: human IL2R beta-gamma Fc (P1AD7029-002)
Running buffer: HBS-EP
T°: 25° C.
Dilution: single injection at 300 nM
Flow: 30 μl/min
Association: 120 sec
Dissociation: 120 sec
Regeneration: 10 mM glycine pH 2.1 for 2×30 sec

Surface plasmon resonance (SPR) experiments were performed on a Biacore T200 with HBS-EP+as running buffer (0.01 M HEPES pH 7.4, 0.15 M NaCl, 0.005% Surfactant P20 (BR-1006-69, Cytiva)). Anti-human Fab specific antibodies (Cytiva 28-9583-25) were directly immobilized by amine coupling on a CM5 chip (Cytiva). The PD1-IL2v variants were captured from supernatants to reach around 200 RU. A single injection of 300 nM recombinant human IL2R beta-gamma Fc was passed over the ligand at 30 μl/min for 120 sec to record the association phase. The dissociation phase was monitored for 120 s and triggered by switching from the sample solution to HBS-EP+. The chip surface was regenerated after every cycle using two injections of 10 mM glycine pH 2.1 for 30 sec. Bulk refractive index differences were corrected for by subtracting the response obtained on the reference flow cell 1. The ratio of response units after binding compared to response units after capture was calculated. In addition the binding curves were visually inspected to identify the variants with faster dissociation rate.

### Affinity of Purified PD1-IL2v Variants to Recombinant Human IL2Rbeta-Gamma-Fc Heterodimer

Instrumentation: Biacore T200
Chip: CM5 (#643)
Fcl to 4: anti-human Fc specific (Roche in house)
Capture: 10 nM PD1-IL2v variants s for 40 s
Analyte: human IL2R beta-gamma Fe (P1AD7029-002)
Running buffer: HBS-EP
T°: 25° C.
Dilution: 5-fold dilution in HBS-EP from 3.2 to 2000 nM
Flow: 30 μl/min
Association: 240 sec
Dissociation: 120 or 600 sec
Regeneration: 10 mM glycine pH 2.1 for 30 sec

Surface plasmon resonance (SPR) experiments were performed on a Biacore T200 with HBS-EP+as running buffer (0.01 M HEPES pH 7.4, 0.15 M NaCl, 0.005% Surfactant P20 (BR-1006-69, Cytiva)). Anti-human Fc specific antibodies (Roche internal) were directly immobilized by amine coupling on a CM5 chip (Cytiva). The PD1-IL2v variants were captured for 40 s at 10 nM. A five-fold dilution series of the human IL2R beta-gamma Fc was passed over the ligand at 30 μl/min for 240 sec to record the association phase. The dissociation phase was monitored for 120 or 600 s and triggered by switching from the sample solution to HBS-EP+. The chip surface was regenerated after every cycle using one injection of 10 mM glycine pH 2.1 for 30 sec. Bulk refractive index differences were corrected for by subtracting the response obtained on the reference flow cell 1. The affinity constants were derived from the kinetic rate constants by fitting to a 1:1 Langmuir binding using the Biaeval software (Cytiva).

### Samples

Following samples were analyzed for binding to human IL2Rbeta-gamma-Fc (Table 6).

TABLE 6
Description of the samples analyzed for
binding to human IL2Rbeta-gamma-Fc.

| Captured molecule | TAPIR ID | Format |
|---|---|---|
| PD1 376 IgG-huIL2v K8S | P1AE0488 | supernatant/purified |
| PD1 376 IgG-huIL2v L12V | P1AE0489 | supernatant/purified |
| PD1 376 IgG-huIL2v L12A | P1AE0490 | supernatant/purified |
| PD1 376 IgG-huIL2v L19V | P1AE0491 | supernatant/purified |
| PD1 376 IgG-huIL2v T51P | P1AE0492 | supernatant |
| PD1 376 IgG-huIL2v H79S | P1AE0493 | supernatant/purified |
| PD1 376 IgG-huIL2v L80A | P1AE0494 | supernatant/purified |
| PD1 376 IgG-huIL2v N88A | P1AE0495 | supernatant/purified |
| PD1 376 IgG-huIL2v N88Q | P1AE0496 | supernatant/purified |
| PD1 376 IgG-huIL2v D109A | P1AE0497 | supernatant/purified |
| PD1 376 IgG-huIL2v Q126N | P1AE0498 | supernatant |
| PD1 376 IgG-huIL2v Q126E | P1AE0499 | supernatant |
| FAP IgG-huIL2v | P1AD1317-002 | purified |
| PD1 379 IgG-huIL2v | P 1AD4370-002 | purified |
| hu IL2R beta-gamma-Fc | P1AD7029-002 | Antigen used as |
| | | analyte, purified |

### Results

Binding Assessment of the PD1-IL2v Variants from Supernatants to Recombinant Human IL2Rbeta-Gamma-Fc Heterodimer

The resonance units after capture and after binding were recorded, the ratio calculated and compared to IL2v without additional mutations (Table 7).

TABLE 7
Resonance units after capture and binding and ratio thereof to identify
variants with reduced binding to human IL2Rbeta-gamma-Fc.

| | injection time | | | | | |
|---|---|---|---|---|---|---|
| | undiluted | | RU | RU | | |
| Binder | supernatant | | captured | binding | ratio | Comment |
| PD1 376 IgG-huIL2v | 30 | s | 148 | 29 | 0.20 | |
| K8S | | | | | | |
| PD1 376 IgG-huIL2v | 30 | s | 216 | 22 | 0.14 | slower association |
| L12V | | | | | | |
| PD1 376 IgG-huIL2v | 30 | s | 160 | 41 | 0.25 | |
| L12A | | | | | | |
| PD1 376 IgG-huIL2v | 30 | s | 161 | 34 | 0.21 | |
| L19V | | | | | | |
| PD1 376 IgG-huIL2v | 120 | s | 142 | 0 | 0.00 | abrogated binding |
| T51P | | | | | | |
| PD1 376 IgG-huIL2v | 30 | s | 208 | 51 | 0.25 | |
| H79S | | | | | | |
| PD1 376 IgG-huIL2v | 60 | s | 194 | 44 | 0.23 | |
| L80A | | | | | | |
| PD1 376 IgG-huIL2v | 60 | s | 179 | 31 | 0.17 | slower association |
| N88A | | | | | | |
| PD1 376 IgG-huIL2v | 30 | s | 167 | 13 | 0.08 | slower association, |
| N88Q | | | | | | faster dissociation |
| PD1 376 IgG-huIL2v | 60 | s | 163 | 37 | 0.23 | |
| D109A | | | | | | |
| PD1 376 IgG-huIL2v | 120 | s | 14 | 0 | 0.00 | low capture level |
| Q126N | | | | | | |
| PD1 376 IgG-huIL2v | 120 | s | 26 | 0 | 0.00 | low capture level |
| Q126E | | | | | | |

| | | | | |
|---|---|---|---|---|
| FAP IgG-huIL2v | 40 s (20 nM) | 256 | 75 | 0.29 |

### Affinity Determination of the PD1-IL2v Variants to Recombinant Human IL2Rbeta-Gamma-Fc Heterodimer

The measure of the affinity to human IL-2Rbeta-gamma shows a very slow off-rate reaching the limit of the instrument and giving unrealistic KDs. However a reduced affinity can still be assessed (Table 8).

TABLE 8
Kinetic constants (1:1 Langmuir binding).

| Binder | TAPIR ID | ka (1/Ms) | kd (1/s) | KD (nM) | Comment |
|---|---|---|---|---|---|
| PD1 376 IgG- | P1AE0488-004 | 1.86E+05 | 3.07E−07 | 0.002 | |
| huIL2v K8S | | | | | |
| PD1 376 IgG- | P1AE0489-007 | 1.91E+05 | 7.53E−07 | 0.004 | |
| huIL2v L12V | | | | | |
| PD1 376 IgG- | P1AE0490-004 | 1.96E+05 | 2.64E−05 | 0.13 | slightly reduced |
| huIL2v L12A | | | | | affinity |
| PD1 376 IgG- | P1AE0491-004 | 1.96E+05 | 3.57E−05 | 0.18 | slightly reduced |
| huIL2v L19V | | | | | affinity |
| PD1 376 IgG- | P1AE0493-004 | 2.1E+05 | 9.2E−06 | 0.05 | |
| huIL2v H79S | | | | | |
| PD1 376 IgG- | P1AE0494-004 | 2.24E+05 | 1.96E−05 | 0.09 | |
| huIL2v L80A | | | | | |
| PD1 376 IgG- | P1AE0495-004 | 1.15E+05 | 1.7E−04 | 1.5 | reduced affinity |
| huIL2v N88A | | | | | |
| PD1 376 IgG- | P1AE0496-004 | 8.18E+04 | 5.85E−03 | 70 | reduced affinity |
| huIL2v N88Q | | | | | |
| PD1 376 IgG- | P1AE0497-004 | 2.16E+05 | 1.87E−05 | 0.09 | |
| huIL2v D109A | | | | | |
| PD1 379 IgG- | P1AD4370-002 | 2.02E+05 | 2.14E−05 | 0.1 | |
| huIL2v | | | | | |

### Conclusion

Twelve variants of IL2v were tested from supernatant to identify candidates with reduced binding to IL2Rbeta-gamma-Fc. Six variants behaved as the parental IL2v (K8S, L12A, L19V, H79S, L80A, D109A), two variants could not be expressed properly (Q126N, Q126E), one variant had slower association and a faster dissociation than IL2v (N88Q), two variants have a slower association than IL2v (N88A, L12V) and one variant lost binding (T51P).

Nine variants were purified and their affinity to IL2Rbeta-gamma-Fc was measured. The N88A and N88Q variants were confirmed having a reduced affinity to human IL2R beta-gamma-Fc. L12V and L19V might be slightly reduced in their affinity to human IL2R beta-gamma-Fc but behaved very close to the parental IL2v.

### Example 1E. Design of Murine Surrogates of Murine PD1-Targeted IL2v Immunoconjugates

In order to facilitate in vivo efficacy studies in mouse models of cancer, murine surrogates of PD1-targeted IL2v immunoconjugates, that target murine PD1, were generated. In order to decrease immunogenicity, all constant antibody domains in these constructs correspond to murine sequences. The V-domain sequences of the anti-murine PD1 antibody, in contrast, have been derived from rat. Due to the cross-reactivity of human IL2v to the murine IL2 receptors, human IL2v has been used in both constructs.

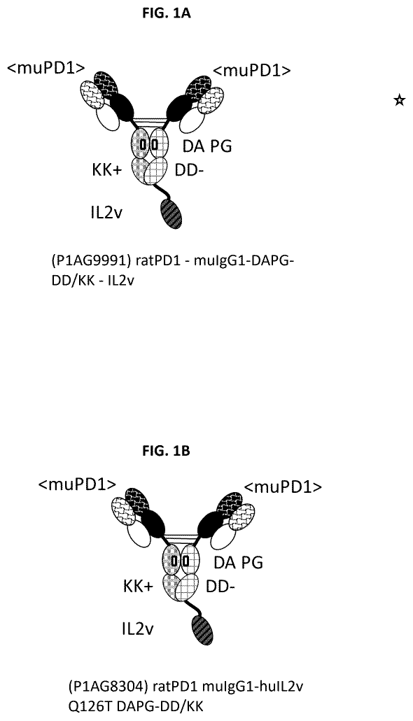

These murine surrogate constructs bind bivalently to murine PD1 via the N-terminal Fab arms on the Fc DD− and Fc KK+ chains whereas the Fc DD− chain additionally carries a C-terminal IL2v (P1AG9991 shown in FIG. 1A) or Q126T mutein thereof (P1AG8304 shown in FIG. 1B). Heterodimerization was achieved by application of complementary charges in the murine IgG1 CH3 domains (Fc DD− and Fc KK+ chain, respectively) and binding to activating Fcγ receptors as well as complement component C1q was abrogated by introduction of DA PG mutations in the murine IgG1 CH2 Fc domains of the antibodies. These immunoconjugates are schematically depicted in FIG. 1A and FIG. B.

### Example 1F. Production and Purification of Murine Surrogates of Murine PD1-Targeted IL2v Immunoconjugates

The murine surrogates of PD1-targeted IL2v immunoconjugates were produced and purified at WuXi Biologics. They were transiently expressed in either HEK293 (P1AG9991, expression system ‘Transient2.0’) or CHO (P1AG8304, expression system ‘Transient2.5’) and purified in a 2-column DSP process: 1. MabSelectSuRe LX affinity chromatography (equilibration and 1st wash: 50 mM Tris-HC, 150 mM NaCl, pH 7.4; 2nd wash: 50 mM Tris-HC, 150 mM NaCl, pH 7.4, 0.1% Triton 100/114; elution: 100 mM Arg, 140 mM NaCl, pH3.4; neutralization: 1 M Arg, pH 9.1); and 2. Superdex200 size-exclusion chromatography (equilibration and formulation buffer: 20 mM Histidine-HCl, 140 mM NaCl, pH 6.0).

For P1AG9991, protein purity was determined by SEC-HPLC (monomer peak 99.3%), non-reducing CE-SDS (main peak 97.8%), reducing CE-SDS (sum of peaks of all 3 different chains 99.9%) and the protein identity was confirmed by LC-MS. The endotoxin level was determined to be 0.34 EU/mg and the final concentration was 1.1 mg/mL. For P1AG8304, protein purity was determined by SEC-HPLC (monomer peak 99.4%), non-reducing CE-SDS (main peak 98.8%), reducing CE-SDS (sum of peaks of all 3 different chains 97.2%) and the protein identity was confirmed by LC-MS. The endotoxin level was determined to be 0.2 EU/mg and the final concentration was 2.1 mg/mL.

### Example 1G. Design of Bispecific Human PD1-/LAG3-Targeted IL2v Immunoconjugates

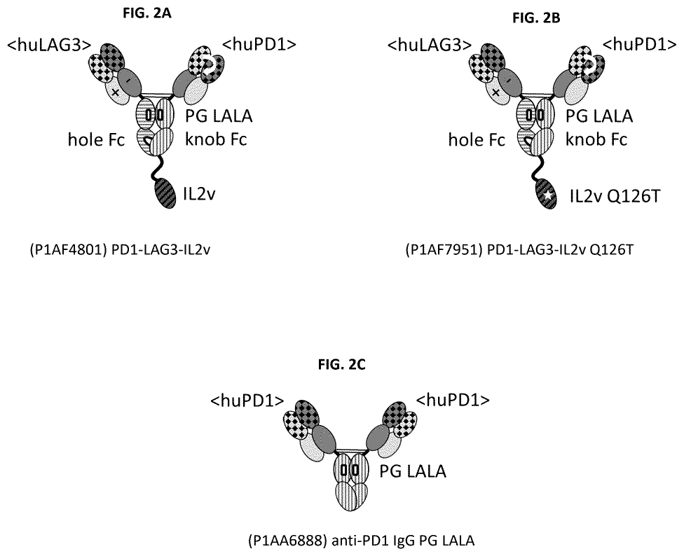

For checkpoint inhibition targeting not only PD1 but also LAG3, bispecific human PD1-/LAG3-targeted IL2v immunoconjugates were generated. These immunoconjugates bind monovalently to human PD1 as well as to human LAG3 via the N-terminal Fab arms. To avoid light chain mispairing, the V-domains of the human PD1-binder were crossed whereas charge complementarity was introduced into the CH1 and Ck domains of the human LAG3 Fab. Heterodimerization of the two heavy chains was achieved by application of the knobs-into-holes technology and binding to activating Fcγ receptors as well as complement component C1q was abrogated by introduction of PG LALA mutations in the CH2 Fc domains of the antibodies. The HC knob chains additionally carry a C-terminal IL2v (P1AF4801) or Q126T mutein thereof (P1AF7951). As a comparator, the monospecific human PD1 IgG1 PG LALA lacking the IL2v cytokine fusion was also generated. These immunoconjugates and the PD1 IgG (P1AA6888) are schematically depicted in FIG. 2A, FIG. 2B, and FIG. 2C.

### Example 1H. Production and Purification of Bispecific Human PD1-/LAG3-Targeted IL2v Immunoconjugates

Production and purification of PTAF4801 was outsourced to Proteros Biostructures GmbH, Martinsried, Germany. HEK293F cells were transiently transfected and the antibody was purified by affinity chromatography (MabSelect Sure) and preparative size-exclusion chromatography. Protein purity was determined by SEC-HPLC (monomer peak >94.2%), non-reducing CE-SDS (main peak >88%), and the protein identity was confirmed by LC-MS. The endotoxin level was determined to be ≤0.5 EU/mg and the final concentration was 2.96 mg/mL. Production and purification of P1AF7951 were done at Roche, Zurich, Switzerland. HEK293 cells were transiently transfected and the antibody was purified by affinity chromatography (MabSelect Sure), cation exchange chromatography (PorosXS) and preparative size-exclusion chromatography. Protein purity was determined by SEC-HPLC (monomer peak 99.1%), non-reducing CE-SDS (main peak 98.7%), and the protein identity was confirmed by LC-MS. The endotoxin level was determined to be ≤0.07 EU/mg and the final concentration was 3.51 mg/mL. Production and purification of P1AA6888 were done at Roche, Penzberg, Germany. HEK Expi293F cells were transiently transfected and the antibody was purified by affinity chromatography (MabSelect Sure) and preparative size-exclusion chromatography (Superdex 200). Protein purity was determined by SEC-HPLC (monomer peak 100%) and non-reducing CE-SDS (main peak 98.1%). The endotoxin level was determined to be <0.16 EU/mL and the final concentration was 5.5 mg/mL.

### Example 2. Selection of IL2v Q126T

### Example 2A. Proliferation of NK92 Cells with PD1-IL2v Variants

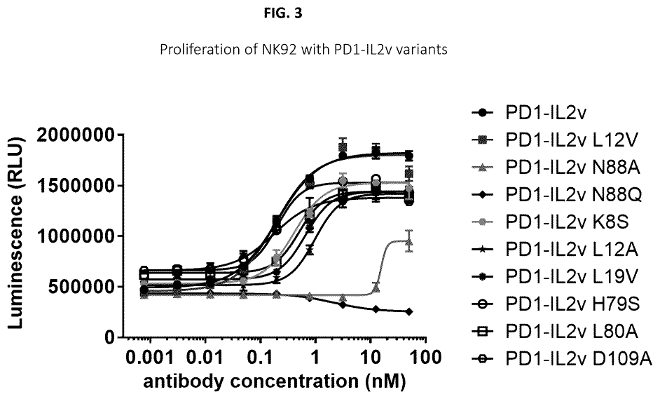

We assessed proliferation of the NK cell line NK92 upon treatment for 3 days with a set of nine newly designed IL2v variants that contained single amino acid exchanges fused to a PD1 antibody and compared their activity to the activity of the parental PD1-IL2v molecule (FIG. 3). The aim was to identify IL2v variants that have a reduced but still detectable activity on the IL2 receptor compared to IL2v in an untargeted setting. The variant N88Q completely lost the activity to induce proliferation of NK92 and the variant N88A had only minimal activity on the IL2 receptor, therefore we did not consider both variants for further evaluation. The variants L12V, H79S and D190A retained a similar activity to the parental IL2v and were therefore not further characterized. The variants K8S, L12A, L19V and L80A had an at least two fold reduced activity on the IL2 receptor and were selected to be characterized in more detail. NK92 proliferation NK92 cells were harvested, counted and assessed for viability. Cells were washed three times with PBS to remove residual IL2 and were re-suspended in medium (RPMI1640, 10% FCS, 1% Glutamine) without TL2. The washed NK92 cells were incubated for two hours in cell incubator (IL2 starvation). After starvation, the cells were re-suspended in fresh medium without IL2 to 200′000 cells per ml. Then, 50 μl of the cell suspension was transferred to each well of a 96-well cell culture treated flat bottom plate and supplemented with 50 μl of the diluted antibodies (in medium without IL2), Proleukin (1.5 pg/ml final concentration) or medium (control wells) to reach a final volume of 100 μl per well. The plate was incubated for 3 days in the incubator. After 3 days the CellTiter-Glo (Promega) reagents and the cell culture plate were equilibrated to room temperature. The CellTiter-Glo solution was prepared as described in the manufacturer's instructions and 100 μl of the solution was added to each well. After 10 min of incubation remaining aggregates were re-suspended by pipetting and 150 μl of the mixture was transferred to a white flat bottom plate. The luminescence was measured with Tecan Spark 10M multimode reader.

### Example 2B. PBMC Proliferation and Activation with PD1-IL2v Variants

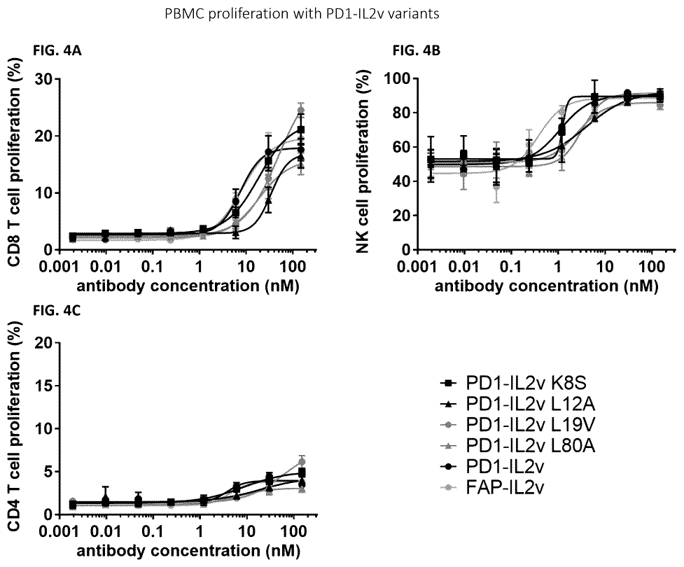

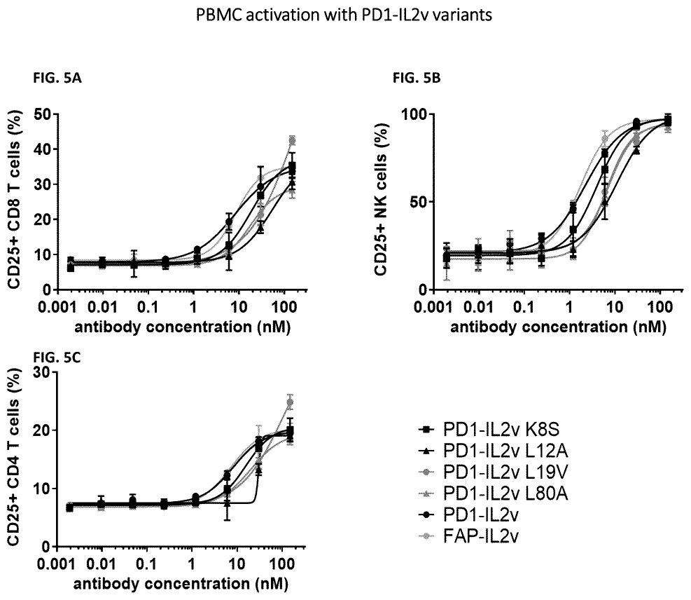

Then we tested the four selected new PD1-IL2v variants (K8S, L12A, L19V and L80A) for their activity on PBMCs and compared them to the parental PD1-IL2v and FAP-IL2v molecules. After five days of treatment with PD1-IL2v variants, PD1-IL2v or FAP-IL2v, proliferation of CD8 T cells, CD4 T cells and NK cells (FIG. 4A, FIG. 4B, and FIG. 4C) and CD25 upregulation (FIG. 5A, FIG. 5B, and FIG. 5C) as a marker of activation of CD8 T cells, NK cells and CD4 T cells was measured by flow cytometry. The results generated with NK92 cells could be confirmed in this experiment; all tested new variants had a reduced activity compared to the two parental IL2v molecules in inducting proliferation and activation of CD8 T cells, CD4 T cells and NK cells. However, the reduction in activity seen with these variants was not considered strong enough. Therefore the characterization of theses variants was not expanded and additional variants were designed.

### Proliferation and Activation of PBMCs

Freshly isolated PBMCs from healthy donors were labeled with CFSE (5(6)-Carboxyfluorescein diacetate N-succinimidyl ester, 21888, Sigma-Aldrich). Briefly, PBMCs were washed once with PBS. In parallel, the CSFE stock solution (2 mM in DMSO) was diluted 1:20 in PBS. PBMCs were resuspended in prewarmed PBS to 1 Mio/ml, 1 μl of the CFSE solution was added to 1 ml cell suspension and the cells were mixed immediately. For an optimal labeling, the cells were incubated for 15 min at 37° C. Then 10 ml prewarmed medium (RPMI1640, 10% FCS, 1% Glutamine) were added to stop the labeling reaction. The cells were spun down for 10 min at 400g and resuspended in ml fresh medium to 1 Mio/ml and incubated for additional 30 min at 37° C. Finally, the cells were washed once with medium and resuspended in fresh medium and used directly or stored overnight in the incubator. The labeled PBMCs were seeded in a 96 well round bottom plate (100′000 cells per well) and treated for 5 days with the indicated molecules. After the incubation the cells were washed once with FACS buffer and stained with 20 μl of a mixture of anti-human CD3 APC-Cy7 (300318, BioLegend), anti-human CD8 APC (344722, BioLegend), anti-human CD56 BV421 (318328, BioLegend) in FACS buffer for 30 min at 4° C. Afterwards PBMCs were washed twice with FACS buffer before fixing them with 1% PFA in FACS buffer and measuring the fluorescence with a BD Fortessa. Proliferation was determined by measuring CFSE dilution of CD8 T cells (CD3+CD8+), CD4 T cells (CD3+CD8−) and NK cells (CD3−CD56+) and activation was determined by Cd25 upregulation on CD8 T cell, CD4 T cells and NK cells.

### Example 2C. NK92 Cell Proliferation with Newly Designed FAP-IL2v Variants

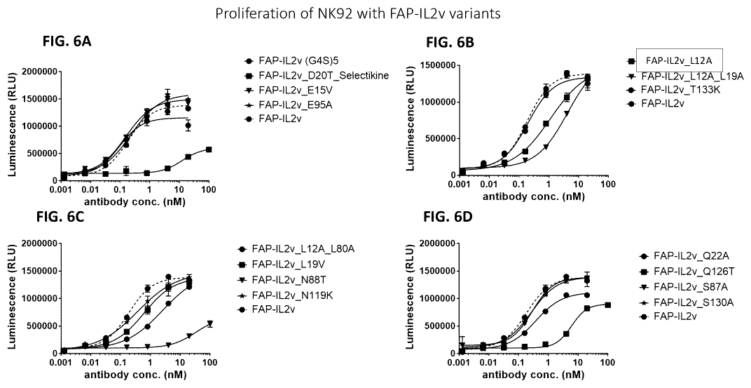

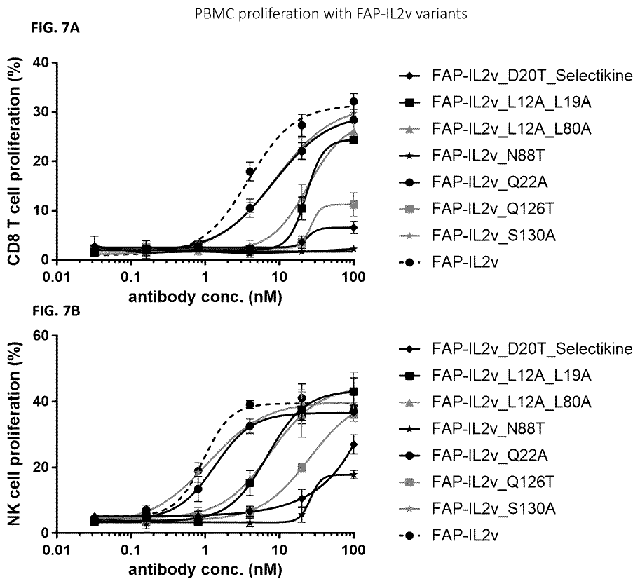

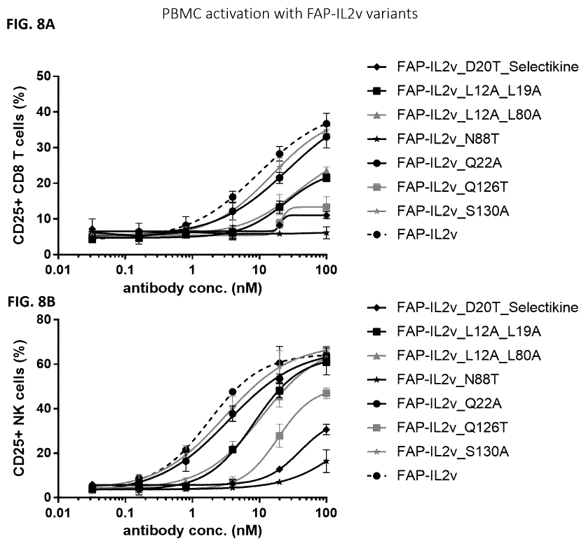

The new IL2v variants and combinations of the previously tested variants were designed to achieve a higher reduction in activity on the IL2 receptor compared to the previously tested IL2v variants. We tested the variants in the FAP-IL2v format and compared their activity to the parental FAP-IL2v molecule. In a first step, proliferation induction of the variants was tested using NK92 cells (FIG. 6A, FIG. 6B, FIG. 6C and FIG. 6D). Proliferation was measured after three days of treatment with the IL2v variants. The IL2v variants D20T_Selectikine, L12A_L19A, L12A_L80A, N88T and Q126T had the strongest reduction in activity compared to the parental IL2v. We selected these variants plus the two variants Q22A and S130A and tested them for their capacity to induce proliferation and activation of CD8 T cells and NK cells within PBMCs. Proliferation (FIG. 7A and FIG. 7B) and CD25 upregulation as a marker for immune cell activation (FIG. 8A and FIG. 8B) was measured after 4 days of treatment. Again, the results obtained with NK92 cells could be confirmed on PBMCs. The variants D20T_Selectikine and N88T had the strongest reduction in activity, which was considered too strong, and these variants were not further evaluated. The other tested variants were evaluated in additional experiments.

### NK92 Proliferation

NK92 cells were harvested, counted and assessed for viability. Cells were washed three times with PBS to remove residual IL2 and were re-suspended in medium (RPMI1640, 10% FCS, 1% Glutamine) without TL2. The washed NK92 cells were incubated for two hours in cell incubator (IL2 starvation). After starvation, the cells were re-suspended in fresh medium without IL2 to 200′000 cells per ml. Then, 50 μl of the cell suspension was transferred to each well of a 96-well cell culture treated flat bottom plate and supplemented with 50 μl of the diluted antibodies (in medium without IL2), Proleukin (1.5 pg/ml final concentration) or medium (control wells) to reach a final volume of 100 μl per well. The plate was incubated for 3 days in the incubator.

After 3 days the CellTiter-Glo (Promega) reagents and the cell culture plate were equilibrated to room temperature. The CellTiter-Glo solution was prepared as described in the manufacturer's instructions and 100 μl of the solution was added to each well. After 10 min of incubation remaining aggregates were re-suspended by pipetting and 150 μl of the mixture was transferred to a white flat bottom plate. The luminescence was measured with Tecan Spark 10M multimode reader.

### Proliferation and Activation of PBMCs

Freshly isolated PBMCs from healthy donors were labeled with cell proliferation dye eFluor670 (65-0840-85, BioLegend). Briefly, PBMCs were washed twice with PBS and resuspended in PBS to a final concentration of 10 Mio cells per ml. In parallel, a 10 μM solution of the Cell Proliferation Dye eFluor 670 was prepared by diluting the stock solution (5 mM) in prewarmed PBS. The PBMCs were mixed and the prediluted Cell Proliferation Dye was added at a 1:1 ratio resulting in a final concentration of 5 μM. PBMCs were incubated for 10 min at 37° C. Afterwards four time the volume of cold medium was added to stop the labeling reaction, the cells were washed three times with medium and resuspended in fresh medium at 1 million cells per ml. The labeled PBMCs were seeded in a 96 well round bottom plate (100′000 cells per well) and treated for 4 days with the indicated molecules. After the incubation the cells were washed twice with PBS, stained with 50 μl/well reconstituted fluorescent reactive live/dead dye (L34976, Invitrogen), and incubated for 20 min at room temperature. Afterwards 150 μl of FACS buffer were added per well and the plate was centrifuged for 4 min at 400 g. The supernatant was removed and the cells were stained with 50 μl of a mixture of CD3 PE-Cy5 (555341, BD Bioscience), CD8 BV711 (301044, BioLegend), CD25 PE-Dazzle 594 (356126, BioLegend), CD56 BV421 (318328, BioLegend) in FACS buffer for 30 min at 4° C. Afterwards PBMCs were washed twice with FACS buffer before fixing them with 1% PFA in FACS buffer and measuring the fluorescence with a BD flow cytometer. Proliferation was determined by measuring proliferation dye dilution of CD8 T cells (CD3+CD8+), and NK cells (CD3−CD56+) and T cell activation by upregulation of CD25 on the respective cells.

### Example 2D. PBMC Proliferation and Activation with Selected FAP-IL2v Variants

Then we tested the newly selected FAP-IL2v variants on PBMCs. Proliferation and activation of CD8 T cells, CD4 T cells and NK cells and STAT5 phosphorylation of CD8 T cells, CD4 T cells, regulatory T cells and NK cells was measured and compared to the activity of the parental FAP-IL2v.

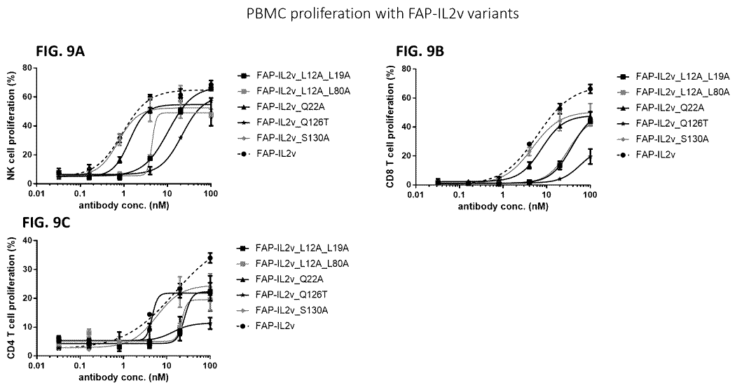

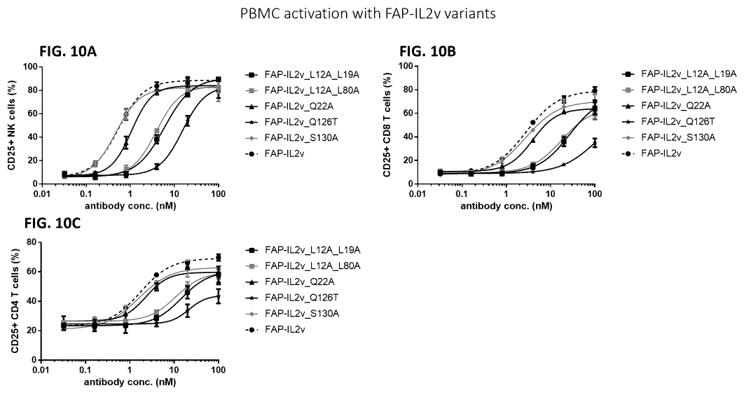

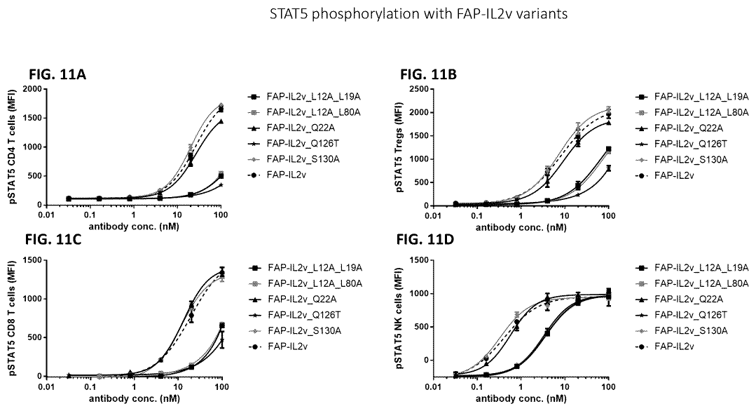

PBMCs were treated for five days with the selected FAP-IL2v variants and the parental FAP-IL2v and proliferation (FIG. 9A, FIG. 9B, and FIG. 9C) and activation (FIG. 10A, FIG. 10B, and FIG. 10C) of immune cells was analyzed. As seen in the previous test, the variants Q22A and S130A behaved similar to the parental IL2v, the variants L12A_L19A and L12A_L80A had an about 10-fold reduced activity and the variant Q126T had a more than 20-fold reduced activity in inducing proliferation and immune cell activation compared to the parental FAP-IL2v. In a second experiment, PBMCs were treated with the same set of FAP-IL2v variants and phosphorylation of STAT5 in CD4 T cells, regulatory T cells, CD8 T cells and NK cells was measured as a direct marker for IL2 receptor activation (FIG. 11A, FIG. 11B, FIG. 11D, and FIG. 11D). As seen before, Q22A and S130A have a similar activity to the parental IL2v. The variants L12A_L19A, L12A_L80A and Q126T have a reduced activity with Q126T been slightly less active than the other two. The IL2v variant Q126T was selected out of all tested IL2v variants as the most promising candidate because the reduction in its activity compared to the parental FAP-IL2v is in the range of what we were aiming for.

### Proliferation and Activation of PBMCs

Freshly isolated PBMCs from healthy donors were labeled with cell proliferation dye eFluor670 (65-0840-85, BioLegend). Briefly, PBMCs were washed twice with PBS and resuspended in PBS to a final concentration of 10 Mio cells per ml. In parallel, a 10 μM solution of the Cell Proliferation Dye eFluor 670 was prepared by diluting the stock solution (5 mM) in prewarmed PBS. The PBMCs were mixed and the prediluted Cell Proliferation Dye was added at a 1:1 ratio resulting in a final concentration of 5 μM. PBMCs were incubated for 10 min at 37° C. Afterwards four time the volume of cold medium was added to stop the labeling reaction, the cells were washed three times with medium and resuspended in fresh medium at 1 million cells per ml. The labeled PBMCs were seeded in a 96 well round bottom plate (100′000 cells per well) and treated for 5 days with the indicated molecules. After the incubation the cells were washed twice with PBS, stained with 50 μl/well reconstituted fluorescent reactive live/dead dye (L34976, Invitrogen), and incubated for 20 min at room temperature. Afterwards 150 μl of FACS buffer were added per well and the plate was centrifuged for 4 min at 400 g. The supernatant was removed and the cells were stained with 50 μl of a mixture of CD3 PE-Cy5 (555341, BD Bioscience), CD4 BV605 (317438, BioLegend), CD8 BV711 (301044, BioLegend), CD25 PE-Dazzle 594 (356126, BioLegend), CD56 BV421 (318328, BioLegend) in FACS buffer for 30 min at 4° C. Afterwards PBMCs were washed twice with FACS buffer before fixing them with 1% PFA in FACS buffer and measuring the fluorescence with a BD flow cytometer. Proliferation was determined by measuring proliferation dye dilution of CD8 T cells (CD3+CD8+), CD4 T cells (CD3+CD4+) and NK cells (CD3−CD56+) and T cell activation by upregulation of CD25 on the respective cells.

### Example 2E. Activity of PD1-IL2v Q126T and FAP-IL2v Q126T

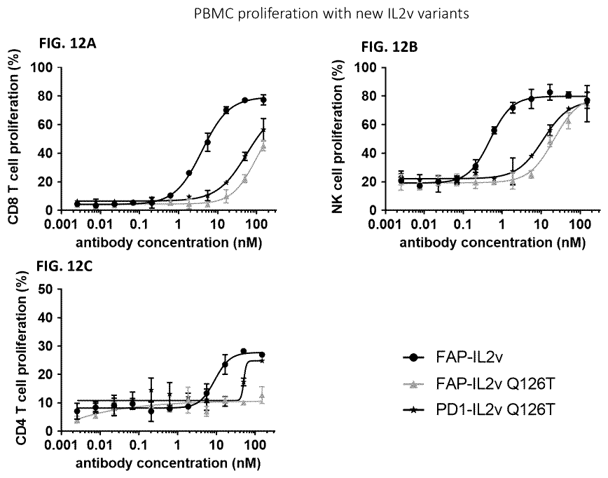

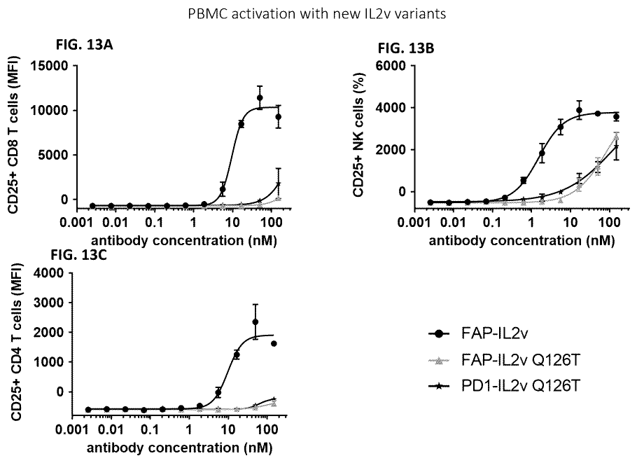

In the next step, PD1-IL2v Q126T and FAP-IL2v Q126T were compared to FAP-IL2v to confirm the results generated so far. PBMCs were treated for five days with the three molecules and proliferation (FIG. 12A, FIG. 12B, and FIG. 12C) and activation (FIG. 13A, FIG. 13B, and FIG. 13C) of CD8 T cells, NK cells and CD4 T cells was determined. PD1-IL2v Q126T and FAP-IL2v Q126T have a comparable activity on CD4 T cells, CD8 T cells and NK cells but the activity is strongly reduced compared to the parental FAP-IL2v.

### Proliferation and Activation of PBMCs

Freshly isolated PBMCs from healthy donors were labeled with cell proliferation dye eFluor670 (65-0840-85, BioLegend). Briefly, PBMCs were washed twice with PBS and resuspended in PBS to a final concentration of 10 Mio cells per ml. In parallel, a 10 μM solution of the Cell Proliferation Dye eFluor 670 was prepared by diluting the stock solution (5 mM) in prewarmed PBS. The PBMCs were mixed and the prediluted Cell Proliferation Dye was added at an 1:1 ratio resulting in a final concentration of 5 μM. PBMCs were incubated for 10 min at 37° C. Afterwards four time the volume of cold medium was added to stop the labeling reaction, the cells were washed three times with medium and resuspended in fresh medium at 1 million cells per ml. The labeled PBMCs were seeded in a 96 well round bottom plate (100,000 cells per well) and treated for 5 days with the indicated molecules. After the incubation the cells were washed twice with PBS, stained with 50 μl/well reconstituted fluorescent reactive live/dead dye (L34957, Invitrogen), and incubated for 15-30 min at room temperature. Afterwards 150 μl of FACS buffer were added per well and the plate was centrifuged for 4 min at 400 g. The supernatant was removed and the cells were stained with 30 μl of a mixture of CD3 BUV359 (563546, BD Bioscience), CD4 PE (300508, BioLegend), CD8 FITC (344704, BioLegend), CD25 PE-Cy7 (302612, BioLegend), CD56 BV421 (318328, BioLegend) in FACS buffer for 30 min at 4° C.

Afterwards PBMCs were washed twice with FACS buffer before measuring the fluorescence with a BD flow cytometer. Proliferation was determined by measuring proliferation dye dilution of CD8 T cells (CD3+CD8+), CD4 T cells (CD3+CD4+) and NK cells (CD3−CD56+) and T cell activation by upregulation of CD25 on the respective cells.

### STAT5 Phosphorylation

Freshly isolated PBMCs from healthy donors were seeded in warm medium (RPMI1640, 10% FCS, 2 mM Glutamine) into a 96 well round bottom plate (200′000 cells/well). The plates were centrifuged at 300 g for 10 min and the supernatant was removed. The cells were re-suspended in 100 μl medium containing the IL2v molecules and stimulated for 20 min at 37° C. To preserve the phosphorylation status, the cells were immediately fixed after stimulation with equal amount of pre-warmed Cytofix buffer (554655, BD Bioscience) for 10 min at 37° C. Afterwards the plates were centrifuged for 5 min at 350 g and the supernatant was removed. To allow intracellular staining, the cells were permeabilized in 100 μl Phosflow Perm buffer III (558050, BD Bioscience) for 30 min at 4° C. Then the cells were washed twice with 150 μl cold FACS buffer and split in two 96 well round bottom plates and stained each with 20 μl of the antibody mix I or II for 60 min in the fridge. Antibody mix I was used to stain pSTAT5 in CD4 T cells and regulatory T cells and antibody mix II was used to stain pSTAT5 in CD8 T cells and NK cells. Afterwards the cells were washed twice with FACS buffer and re-suspended in 200 μl FACS buffer containing 2% PFA per well. The analysis was performed using a BD flow cytometer gating on CD8 T cells (CD3+CD8+), NK cells (CD3−CD56+, CD4 T cells (CD4+) and Tregs (CD4+CD25+FoxP3+).

TABLE 9
FACS antibody mix I (CD4 T cells and regulatory T cells)

| | Volume/ |
|---|---|
| Antibody | sample |
| CD4 PE/Cy7, clone SK3, mouse IgG1, κ (557852, | 0.5 μl/well |
| BD Bioscience) | |
| CD25 APC, clone M-A251, mouse IgG1, κ (356110, | 0.5 μl/well |
| BioLegend) | |
| PE Mouse anti-Human FoxP3 Clone 259D/C7 (560046, BD | 0.5 μl/well |
| Bioscience) | |
| A488 pSTAT5 (pY694), clone 47, mouse IgG1 (562075, BD | 0.5 μl/well |
| Bioscience) | |

TABLE 10
FACS antibody mix II (CD8 T cells and NK cells)

| | Volume/ |
|---|---|
| Antibody | sample |
| CD3 PE/Cy7, clone UCHT1, mouse IgG1, κ (300420, | 0.5 μl/well |
| BioLegend) | |
| CD56 APC, clone HCD56, mouse IgG1, κ (318310, | 0.5 μl/well |
| BioLegend) | |
| CD8 PE, clone HIT8a, mouse IgG1 (555635, BD Bioscience) | 0.5 μl/well |
| A488 pSTAT5 (pY694), clone 47, mouse IgG1 (562075, BD | 0.5 μl/well |
| Bioscience) | |

### Example 3. PD1-IL2v Q126T and PD1-LAG3-IL2v Q126T

### Example 3A. Assays

### IL-2R Signaling Assay

In one aspect, an assay is provided to determine the potency and cis/trans-signaling of a PD-1-IL-2v immunoconjugate (e.g., including at least one binding domain that binds to PD-1 conjugated to an IL-2 polypeptide with additional mutations) is provided.

For this purpose, CD4 T cells from healthy donor PBMCs are sorted with CD4 beads (Miltenyi, #130-045-101) and activated for 3 days in presence of 1 μg/ml plate-bound anti-CD3 (overnight pre-coated, clone OKT3, #317315, BioLegend) and 1 μg/ml of soluble anti-CD28 (clone CD28.2, #302923, BioLegend) antibodies to induce PD-1 expression. Three days later, the cells are harvested and washed several times to remove endogenous cytokines and half of the cells are labeled with Cell Trace Violet (CTV) (5 μM, 5 minutes at room temperature (RT); C34557, Thermo Scientific) and the other half are left unlabeled.

Then, the unlabelled cells are incubated with a saturating concentration of a competing anti-PD-1 antibody (in-house molecule, 10 μg/ml) for 30 minutes at RT followed by several washing steps to remove the excess unbound anti-PD-1 antibody. Thereafter, the PD-1 pre-blocked cells (25 μl, 6*10⁶ cells/ml) are co-cultured 1:1 with the PD-1 CTV-labeled cells (25 μl, 6×10⁶ cells/ml) in a V-bottom plate before being treated for 12 minutes at 37° C. with increasing concentrations of treatment immunoconjugates (50 μl, 1:10 dilution steps). To preserve the phosphorylation state, an equal amount of Phosphoflow Fix Buffer I (100 μl, 557870, BD Bioscience) is added after 12 minutes incubation with the various constructs. The cells are then incubated for an additional 30 minutes at 37° C. before being permeabilized overnight at −80° C. with Phosphoflow PermBuffer TTT (558050, BD Bioscience). On the next day, STAT-5 in its phosphorylated form is stained for 30 minutes at 4° C. by using an anti-STAT-5P antibody (47/Stat5(pY694) clone, 562076, BD Bioscience).

The cells are acquired at the fluorescence-activated cell sorting (FACS) BD-LSRFortessa (BD Bioscience) instrument. The frequency of STAT-5P is determined with FlowJo (V10) and plotted with GraphPad Prism (v8).

The dose-response curves on PD-1⁺ T cells provide information on the potency of the assessed molecules in signaling through the IL-2R. In addition, the dose-response curves on T cells pre-treated with a competing anti-PD-1 antibody, to prevent the PD-1 mediated delivery, show the potency of the molecules in providing IL-2R signaling independently from PD-1 expression.

### CMV-Specific Restimulation Assay

In the context of chronic viral infections, it has been developed an in-vitro assay to evaluate the effect of PD-1 targeting to deliver mutated versions of IL-2v to dysfunctional antigen-specific T cells. To avoid restrictions on the amount of suitable donors for the assay, a CMV immunogenic viral-protein (pp65) is used as re-call antigen for T cells given that roughly 80% of the population is CMV-seropositive. Hence, healthy human donor peripheral blood mononuclear cells (PBMCs) are stimulated with CMV-pp65 (Miltenyi, #130-093-435) in presence of the different constructs at the concentration of 0.6 nM. Fourtythree hours later the protein transport from the Golgi is blocked by adding Protein Transport Inhibitors (GolgiPlug™ #555029, BD Bioscience; and GolgiStop™ #554724, BD Bioscience) followed by an incubation of the cells at 37° C. for 5 additional hours. The cells are then washed, stained on the surface with anti-human CD3, CD4, CD8, CD62L and CD45RO antibodies before being fixed/permeabilized with the FoxP3 Transcription Factor Staining Buffer Set (eBioscience). At last an intracellular staining for IL-2, IFN-γ and Ki67 (all from eBioscience) is performed to measure cytokine secretion and cell proliferation.

### Suppression Assay

In one aspect, an assay is provided to assess whether PD-1-IL-2v immunoconjugates can reverse the regulatory T cell (T_{reg}) suppression of conventional T cell (T_{cov}) effector functions. In some instances, T_{conv} and T_{reg} are isolated and labeled.

In certain aspects, CD4⁺ CD25⁺ CD127^{dim} T_{reg} are isolated with the two-step Regulatory T cell Isolation Kit (Miltenyi, #130-094-775). In parallel, the CD4⁺ CD25⁻ T_{conv} are isolated by collecting the negative fraction of a CD25 positive selection (Miltenyi, #130-092-983) followed by a CD4⁺ enrichment (Miltenyi, #130-045-101). The T_{conv} are labeled with Carboxyfluoroscein succinimidyl ester (CFSE, eBioscience, #65-0850-84) and the T_{reg} are labeled with Cell Trace Violet (CTV, ThermoFisher scientific, C34557) to be able to distinguish them and to track the proliferation of both populations. T_{conv} and T_{reg} are then cultured together for 5 days, with or without treatment, in presence of CD4⁻ CD25⁻ PBMCs from an unrelated donor to provide an allospecific stimulation.

In certain aspects, on day 5, the accumulation of cytokines in the Golgi complex is enhanced by applying Protein Transport Inhibitors (GolgiPlug™ #555029, BD Bioscience; and GolgiStop™ #554724, BD Bioscience) for 5 hours prior to the FACS staining. The ability of the proliferated T_{conv} to secrete granzyme B (GrzB) in presence and absence of T_{reg} is measured. T_{reg} suppression is calculated with the following formula:

$$\% cytokine suppression = 100 - ( \frac{\% {cytokine}_{( Tconv + Treg \pm immunoconjugate )}}{\% {cytokine}_{( Tconv )}} * 100 )$$

Where % cytokine_{(Tconv+Treg±immunoconjugate)} is the level of cytokine secreted by T_{conv} in the presence of T_{reg}±treatment immunoconjugate and % cytokine_{(Tconv)} is the level of cytokine secreted by T_{conv} in the absence of T_{reg}. P is calculated using one-way ANOVA (*p<0.05, **p<0.01, ***p<0.001, ****p<0.0001).

### Internalization Assay

In one aspect, an assay is provided to assess the internalization of the different immunoconjugates. For this purpose PBMCs are isolated by density gradient centrifugation using Ficoll-Paque (Sigma-Aldrich). CD4 T cells are bead-sorted starting from 10⁸ PBMCs by using a CD4-positive selection kit (Miltenyi, #130-045-101) following manufacturer instructions. CD4 T cells are then seeded in a 12 well plate previously coated with 1 μg/ml anti-CD3 (overnight pre-coated, clone OKT3, #317315, BioLegend) at 2-4×10⁶ cells/well in RPMI 10% FBS in presence of 1 μg/ml of soluble anti-CD28 (clone CD28.2, #302923, BioLegend) and cultured for 3 days at 37° C.

Three days activated CD4 T cells are incubated in duplicates in FACS tubes in presence the immunoconjugates for 30 minutes at 4° C. The cells are then washed, divided in two groups, one of which is incubated for 3 additional hours at 37° C. and the other one is immediately stained with a PE labelled anti-PGLALA secondary antibody and anti-CD4 antibody (eBioscience) before being fixed with BD Cell Fix. After the 3 hours incubations, also the second group of the cells is stained with the PE labelled anti-PGLALA secondary antibody and anti-CD4 before fixation. The cells are then acquired at LSRFortessa (BD Biosciences) and data analysis are performed with FlowJo (V10) and plotted with GraphPad Prism (v8). The expression levels of detectable antibody on the cell surface at 4° C. is compared to the one at 37° C. to calculate the percentage of internalized molecules at 37° C. P is calculated using one-way ANOVA (*p<0.05, **p<0.01, ***p<0.001, ****p<0.0001).

### Example 3B. IL-2R Signaling (STAT5-P) on Activated PD-1⁺ and PD-1⁻ CD4 T Cells Upon Treatment with Increasing Doses of PD-1-IL-2v Immunoconjugates

The potency and the cis/trans-signaling of a PD-1-IL-2v immunoconjugates were measured as IL-2R signaling by treating activated PD-1 expressing (PD-1⁺) and PD-1 negative (PD-1⁻) (anti-PD-1 pre-treated) CD4 T cells with increasing concentrations of immunoconjugates. The purpose was to determine the dependency of the PD-1-IL-2v immunoconjugates on the PD-1 expression of the T cells in order to deliver IL-2R signaling.

For this CD4 T cells from healthy donor PBMCs were sorted with CD4 beads (#130-045-101, Miltenyi) and activated for 3 days in presence of 1 μg/ml plate-bound anti-CD3 (overnight pre-coated, clone OKT3, #317315, BioLegend) and 1 μg/ml of soluble anti-CD28 (clone CD28.2, #302923, BioLegend) antibodies to induce PD-1 expression. Three days later, the cells were harvested and washed several times to remove endogenous cytokines and half of the cells were labeled with Cell Trace Violet (CTV) (5 μM, 5 minutes at room temperature (RT); C34557, Thermo Scientific) and the other half were left unlabeled.

Then, the unlabeled cells were incubated with a saturating concentration of a competing anti-PD-1 antibody (in-house molecule, 10 μg/ml) for 30 minutes at RT followed by several washing steps to remove the excess unbound anti-PD-1 antibody. Thereafter, the PD-1 pre-blocked unlabeled cells (25 μl, 6*10⁶ cells/ml) were co-cultured 1:1 with the PD-1⁺ CTV-labeled cells (25 μl, 6×10⁶ cells/ml) in a V-bottom plate before being treated for 12 minutes at 37° C. with increasing concentrations of treatment immunoconjugates (50 μl, 1:10 dilution steps). To preserve the phosphorylation state, an equal amount of Phosphoflow Fix Buffer I (100 μl, 557870, BD Bioscience) was added after 12 minutes incubation with the various constructs to allow the IL-2R signaling upon binding to PD-1. The cells were then incubated for an additional 30 minutes at 37° C. for fixation before being permeabilized overnight at −80° C. with Phosphoflow PermBuffer III (558050, BD Bioscience). On the next day, STAT-5 in its phosphorylated form was stained for 30 minutes at 4° C. by using an anti-STAT-5P antibody (47/Stat5(pY694) clone, 562076, BD Bioscience).

The cells were acquired at the flow cytometer (FACS) BD-LSRFortessa (BD Bioscience) instrument. The frequency of STAT-5P was determined with FlowJo (V10) and plotted with GraphPad Prism (v8).

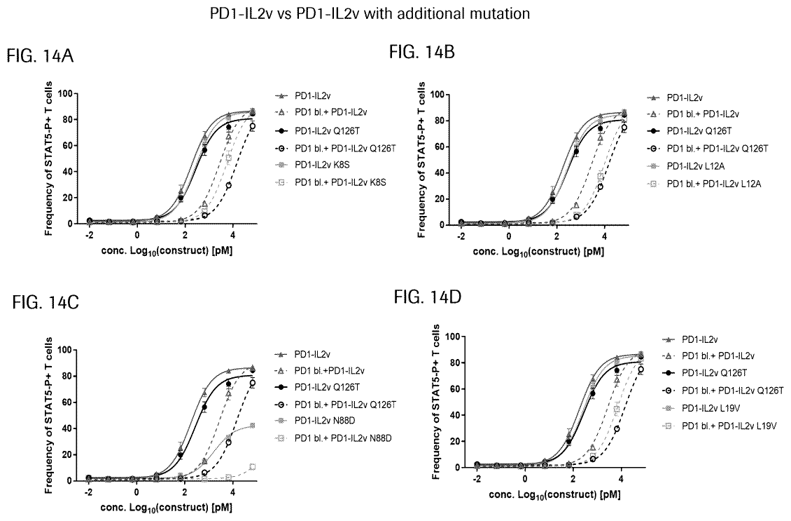

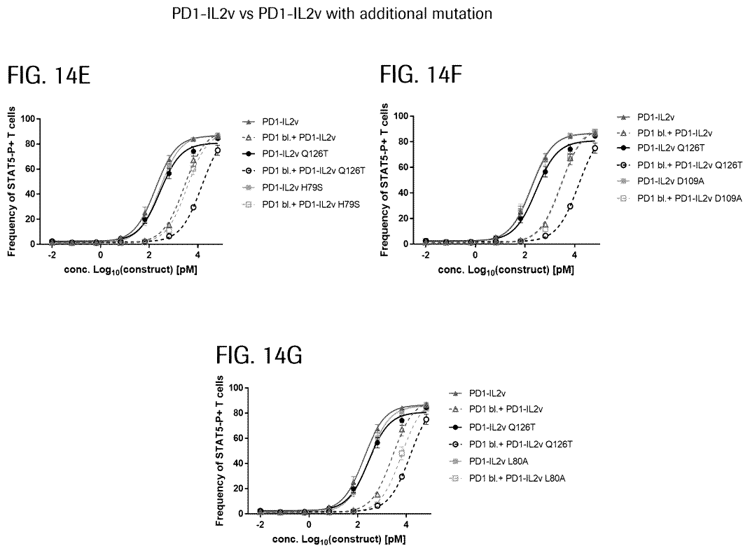

The data in FIG. 14A, FIG. 14B, FIG. 14C, FIG. 14D, FIG. 14E, FIG. 14F, and FIG. 14G show the potency difference of selected PD1-IL2 variants in signaling through the IL-2R on PD-1⁺ and PD-1⁻ CD4 T cells. The potency measurement in the PD1⁺ CD4 T cells reflects the PD1-mediated delivery of IL-2v versus the PD1-independent delivery of IL-2v in PD1⁻ CD4 T cells. In Table 11, the STAT-5P EC50 fold increase between PD1-mediated and PD-1 independent delivery of IL-2v of each PD1-IL2v immunoconjugate molecule was calculated by dividing the EC50 of the PD-1 pre-blocked cells by the EC50 of PD1⁺ T cells. This provides evidence on the strength of the PD1-dependent delivery of IL2v for each of the IL2v mutants. Furthermore, the EC50 fold increase between the various PD1-IL2v immunoconjugates and PD1-IL2v was calculated by dividing the EC50 of the new mutants by the EC50 of PD1-IL2v. This indicated the loss in potency of the PD1-IL2v immunoconjugates in signaling thorugh the IL-2R due to their reduced affinity.

TABLE 11
EC50 and Area under the Curve (AUC) of the dose-response STAT-5 phosphorylation
for the each mutant on PD-1⁺ and PD-1⁻ CD4 T cells obtained from 4 donors.

| | | | Cis- | | | |
|---|---|---|---|---|---|---|
| | | | delivery: | | | |
| | | | fold | | | |
| | | EC50 | increase in | | | AUC |
| | EC50 | (Mean ± | EC50 | | AUC | (Mean ± |
| | (Mean ± | SEM) | EC50 [with | | (Mean ± | SEM) |
| | SEM) | on PD1- | PD1bl]/ | Potency | SEM) on | on PD1- |
| | on PD1+ T | blocked | EC50 | reduction vs | PD1⁺ | blocked |
| Compounds | cells | Tcells | [PD1⁺] | PD1-IL2v | Tcells | Tcells |
| PD1-IL2v | 200.7 ± | 2896.5 ± | 18.9 ± | 1 | 221 ± | 123.9 ± |
| | 57 | 336.9 | 5.7 | | 7.7 | 6 |
| PD1-IL2v | 298.8 ± | 6184.5 ± | 26.7 ± | 1.488789238 | 200.7 ± | 99.7 ± |
| K8S | 75.4 | 1129.8 | 8.7 | | 5.7 | 6.3 |
| PD1-IL2v | 299.1 ± | 10820.8 ± | 44.5 ± | 1.490284006 | 197.4 ± | 84.1 ± |
| L12A | 66.4 | 1786.8 | 13.3 | | 5.8 | 5.4 |
| PD1-IL2v | 290.2 ± | 7926.5 ± | 36 ± | 1.445939213 | 201.2 ± | 92.7 ± |
| L19V | 78.9 | 1399.7 | 11.5 | | 6.9 | 6.3 |
| PD1-IL2v | 304 ± | 3860 ± | 16.9 ± | 1.514698555 | 203.8 ± | 111.8 ± |
| H79S | 82.4 | 400.7 | 5.5 | | 6.9 | 5.7 |
| PD1-IL2v | 317.7 ± | 6863.3 ± | 29.1 ± | 1.582959641 | 197.6 ± | 94.9 ± |
| L80A | 78.2 | 1006.6 | 10.5 | | 7.4 | 6.4 |
| PD1-IL2v | 1610.7 ± | 76444533.5 ± | 69119.9 ± | 8.025411061 | 47.1 ± | 7 ± |
| N88Q | 350 | 65178049.8 | 56839.3 | | 4.2 | 1.6 |
| PD1-IL2v | 224.2 ± | 3442.5 ± | 24.3 ± | 1.117090184 | 216 ± | 117.6 ± |
| D109A | 79.7 | 414.5 | 9.2 | | 8 | 6.5 |
| PD1-IL2v | 1719.3 ± | 79155371.5 ± | 47113.8 ± | 8.56651719 | 67.2 ± | 9.5 ± |
| N88D | 274.7 | 54084723.4 | 33507.8 | | 3.7 | 1.8 |
| PD1-IL2v | 315.5 ± | 14479.5 ± | 56.5 ± | 1.571998007 | 188.4 ± | 71.2 ± |
| Q126T | 93.6 | 719.3 | 12.8 | | 10.4 | 2.9 |
| FAP-IL2v | 1630.8 ± | 1788.5 ± | 1.1 ± | 8.125560538 | 144.3 ± | 142.3 ± |
| | 34.9 | 131.4 | 0.1 | | 3.3 | 4.6 |

In this particular assay, some further mutated immunoconjugates show to have similar potency as PD1-IL2v in signaling through the IL-2R on PD-1⁺ T cells but have a reduced activity on PD-1⁻ T cells like Q126T and L12A which have a cis-activity of 56.5 and 44.5 folds respectively. Others like N88D and N88Q have also a reduced activity in signaling through the IL-2R on PD-1⁺ T cells while others maintain the same characteristics of PD1-IL2v (FIG. 14A, FIG. 14B, FIG. 14C, FIG. 14D, FIG. 14E, FIG. 14F, and FIG. 14G). Table 11 shows the EC50 and Area under the Curve (AUC) of the dose-response STAT-5 phosphorylation for the each mutant on PD-1⁺ and PD-1⁻ CD4 T cells obtained from 4 donors.

### Example 3B. IL-2R Signaling (STAT5-P) on Activated PD-1⁺ CD4 T Cells Upon Treatment with Increasing Doses of PD-1-IL-2v, FAP-IL2v and NKG2D-IL2v Immunoconjugates

In this experiment, the STAT5 phosphorylation was used as readout to assess the potency difference in signalling through the IL-2R upon binding to PD-1 on PD-1 expressing CD4 T cells of PD1-IL2vQ126T, FAP-IL2vQ126T and NKG2D-IL2vQ126T versus PD1-IL2v and FAP-IL2v in a dose dependent manner.

For this purpose CD4 T cells were sorted from healthy donor PBMCs with CD4 beads (#130-045-101, Miltenyi) and activated for 3 days in presence of 1 μg/ml plate bound anti-CD3 (overnight pre-coated, clone OKT3, #317315, BioLegend) and 1 μg/ml of soluble anti-CD28 (clone CD28.2, #302923, BioLegend) antibodies to induce PD-1 expression. Three days later, the cells were harvested and washed several times to remove endogenous IL-2. The cells (50 μl, 4*106 cells/ml) were seeded into a V-bottom plate before being treated for 12 min at 37° C. with increasing concentrations of treatment antibodies (50 μl, 1:10 dilution steps with the top concentration of 66 nM). To preserve the phosphorylation state, an equal amount of Phosphoflow Fix Buffer I (100 μl, 557870, BD) was added right after 12 minutes incubation with the various constructs. The cells were then incubated for additional 30 min at 37° C. before being permeabilized overnight at 80° C. with Phosphoflow PermBuffer III (558050, BD). On the next day STAT-5 in its phosphorylated form was stained for 30 min at 4° C. by using an anti-STAT-5P antibody (47/Stat5(pY694) clone, 562076, BD).

The cells were acquired at the FACS BD-LSR Fortessa (BD Bioscience). The frequency of STAT-5P were determined with FlowJo (V10) and plotted with GraphPad Prism (V8).

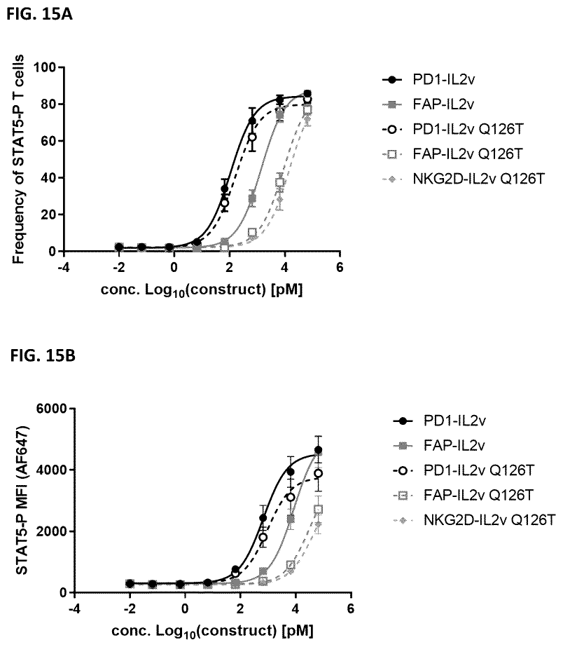

The data in FIG. 15A and FIG. 15B show the potency difference of selected PD1−, FAP− and NKG2D-IL2 variants in PD-1⁺ CD4 T cells. The potency measurement in the PD1⁺ CD4 T cells reflects the PD1-mediated delivery of IL-2v versus the PD1-independent delivery of the FAP-IL-2v and FAP-IL2vQ126T.

Table 12 shows the EC50 of the dose-response STAT-5 phosphorylation as frequencies and mean fluorescent intensity (MFI) for the IL-2v mutants on PD-1⁺ CD4 T cells obtained from 4 donors.

This experiment shows the PD-1 dependent and independent delivery of the IL-2 mutants to the IL-2R. The FAP-targeting antibodies behave in this experiment like an untargeted IL2v because of lack of FAP expression by activated CD4 T cells. The untargeted IL2vQ126T is 6.5 times less potent than the untargeted IL2v, whereas the PD1-IL2vQ126T is only 1.5 times less potent than the PD1-IL2v. These findings predict less IL-2 mediated off-target effects, while having similar IL-2R signaling on PD1⁺ Tcells by PD1-IL2vQ126T. NKG2D-IL2vQ126T has a reduced potency on activated CD4 T cells as FAP-IL2vQ126T (FIG. 15A and FIG. 15B).

TABLE 12
(A-B) EC50 of the dose-response STAT-5 phosphorylation as
frequencies and mean fluorescent intensity (MFI) for the IL-2v mutants
on PD-1⁺ CD4 T cells. Mean ± SEM of 4 donors.

| | | Potency | | Potency |
|---|---|---|---|---|
| | | reduction | | reduction |
| | EC50 on | versus | EC50 | versus |
| Compounds | Frequency | PD1-IL2v | on MFI | PD1-IL2v |
| PD1-IL2v | 109.9 | 1 | 675.9 | 1 |
| FAP-IL2v | 1413 | 12.85714286 | 8528 | 12.61725107 |
| PD1-IL2v | 165 | 1.501364877 | 937.7 | 1.387335405 |
| Q126T | | | | |
| FAP-IL2v | 9020 | 82.07461328 | 30228 | 44.7225921 |
| Q126T | | | | |
| NKG2D-IL2v | 14734 | 134.0673339 | 42198 | 62.43231247 |
| Q126T | | | | |

### Example 3C. Expansion of CMV-Specific CD4 T Cell Effector Function Upon Treatment with PD1-IL2v Immunoconjugates

To assess the ability of PD-1 targeted and untargeted IL-2v and IL-2vQ126T to expand antigen specific CD4 T cell response in the context of chronic viral infections, a CMV immunogenic viral-protein (pp65) was used as re-call antigen. Hence, healthy human donor peripheral blood mononuclear cells (PBMCs) were stimulated with CMV-pp65 (#130-093-435, Miltenyi) in presence of the different constructs at the concentration of 0.6 nM. Fourtythree hours later the protein transport from the Golgi was blocked by adding Protein Transport Inhibitors (GolgiPlug™ #555029, BD Bioscience; and GolgiStop™ #554724, BD Bioscience) followed by an incubation of the cells at 37° C. for additional 5 hours. The cells were then washed, stained on the surface with anti-human CD3, CD4, CD8, CD62L and CD45RO antibodies before being fixed/permeabilized with the FoxP3 Transcription Factor Staining Buffer Set (eBioscience). At last an intracellular staining for IL-2, IFN-γ and Ki67 (all from eBioscience) was performed to measure cytokine production and cell proliferation.

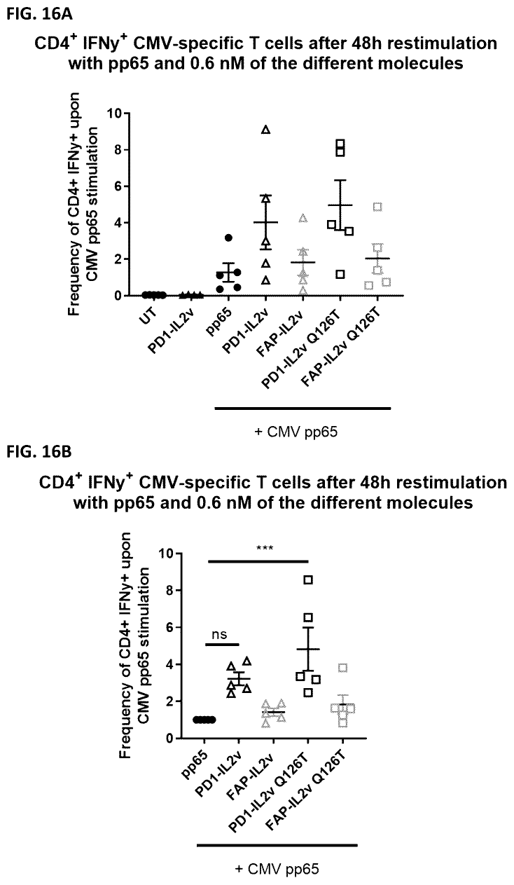

The cells were acquired at the FACS BD-LSR Fortessa (BD Bioscience). The frequency of IFN-γ+ cells were determined with FlowJo (V10) and plotted with GraphPad Prism (v8). P is calculated using one-way ANOVA (*p<0.05, **p<0.01, ***p<0.001, ****p<0.0001) This experiment shows that PD1-IL2v and PD1-IL2vQ126T increase the frequencies of CMV-specific CD4 T cells when compared to pp65 alone or in combination with FAP-IL2v and FAP-IL2vQ126T (FIG. 16A). It also shows that PD1-IL2vQ126T increases of roughly 5 folds the frequencies of CMV-specific CD4 T cells secreting IFN-γ after 48 hours from restimulation and it is significantly better than pp65 stimulation alone (FIG. 16B).

Table 13 depicts the frequency of CMV specific CD4 T cells upon restimulation with the CMV protein pp65 and fold increase in the frequencies of CMV-specific CD4 T cells elicited by the indicated treatment in combination with pp65.

TABLE 13
Frequency of CMV specific CD4 T cells upon restimualtion with
the CMV protein pp65 and fold increase in the frequencies
of CMV-specific CD4 T cells elicited by the indicated treatment
in combination with pp65. Mean ± SEM of 5 donors.

| | | Fold increase in |
|---|---|---|
| | Frequency of CMV-pp65 | frequency of CMV-pp65 |
| Compounds at | specific T cells upon | specific T cells |
| 0.6 nM | 48 h of restimulation | relative to pp65 alone |
| pp65 | 1.274 | 1 |
| PD1-IL2v | 4.018 | 3.219904373 |
| FAP-IL2v | 1.8168 | 1.410867243 |
| PD1-IL2v Q126T | 4.962 | 4.822097026 |
| FAP-IL2v Q126T | 2.04 | 1.82568448 |

### Example 3D. Rescue of T_{conv} Effector Function from T_{reg} Suppression Upon Treatment with PD1-IL2v Immunoconjugates

To assess the ability to rescue conventional T cells (Tconv) from Treg suppression, a suppressive-function assay was established where Tconv and Treg were cultured together for 5 days with CD4⁻ CD25⁻ from an unrelated donor for allospecific stimulation in presence or absence of immunoconjugates.

In certain aspects, CD4⁺ CD25⁺ CD127^{dim} T_{reg} were isolated with the two-step Regulatory T cell Isolation Kit (Miltenyi, #130-094-775). In parallel, the CD4⁺ CD25⁻ T_{conv} were isolated by collecting the negative fraction of a CD25 positive selection (Miltenyi, #130-092-983) followed by a CD4⁺ enrichment (Miltenyi, #130-045-101). The T_{conv} were labeled with Carboxyfluoroscein succinimidyl ester (CFSE, eBioscience, #65-0850-84) and the T_{reg} were labeled with Cell Trace Violet (CTV, ThermoFisher scientific, C34557) to be able to distinguish them and to track the proliferation of both populations. T_{conv} and T_{reg} were then cultured together for 5 days, with or without treatment, in presence of CD4⁻ CD25⁻ PBMCs from an unrelated donor to provide an allospecific stimulation.

In certain aspects, on day 5, the accumulation of cytokines in the Golgi complex was enhanced by applying Protein Transport Inhibitors (GolgiPlug™ #555029, BD Bioscience; and GolgiStop™ #554724, BD Bioscience) for 5 hours prior to the FACS staining. The cells were acquired at the FACS BD-LSR Fortessa (BD Bioscience). Data analysis were performed with FlowJo (V10) and plotted with GraphPad Prism (v8).

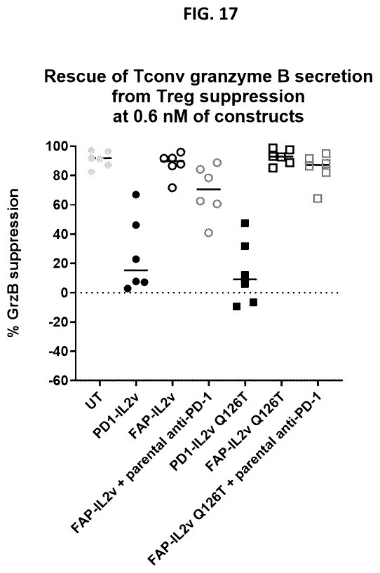

The ability of the proliferated T_{conv} to secrete granzyme B (GrzB) in presence and absence of T_{reg}was measured. T_{reg} suppression is calculated with the following formula:

$$\% cytokine suppression = 100 - ( \frac{\% {cytokine}_{( Tconv + Treg \pm immunoconjugate )}}{\% {cytokine}_{( Tconv )}} * 100 )$$

Where % cytokine_{(Tconv+Treg±immunoconjugate)} is the level of cytokine secreted by T_{conv} in the presence of T_{reg}±treatment immunoconjugate and % cytokine(Tconv) is the level of cytokine secreted by T_{conv} in the absence of T_{reg}. P is calculated using one-way ANOVA (*p<0.05, **p<0.01, ***p<0.001, ****p<0.0001) FIG. 17 shows the median and the individual values of 10 donors from independent experiments and table 14 depicts the numerical median values.

The data in FIG. 17 show that Treg suppress 91% of granzyme B secretion by Tconv when left untreated. PD1-IL2v and PD1-IL2vQ126T at 0.6 nM reduce the suppression to 25.6% and 13.6%, respectively, and therefore are able to rescue 74% and 86% of granzyme B secretion by Tconv from Treg suppression (FIG. 17 and Table 14). 0.6 nM of the untargeted versions, FAP-IL2v and FAP-IL2vQ126T, reduce the suppression to 87.6% and 92.7% respectively and therefore are able to rescue only 12.3% and 7.2% of the granzyme B secretion (FIG. 17 and Table 14). The additional combination of the untargeted FAP-IL2v and FAP-IL2vQ126T with 66 nM of the parental blocking anti-PD-1 antibody modestly decreases the Treg suppression to 69.2% and 84.5%, respectively, resulting in the rescuing of 30.7% and 15.5% of granzyme B secretion by T conv (FIG. 17 and Table 14).

TABLE 14
Percentage of Treg-mediated suppression of granzyme
B produced by Tconv and percentage of rescued granzyme
B production by T conv. Median of 6 donors.

| | Percentage of | Percentage of |
|---|---|---|
| Compounds at | suppressed Granzyme B | rescued Granzyme B |
| 0.6 nM | production by T conv | production by T conv |
| UT | 91.31001 | 8.689995 |
| PD1-IL2v | 25.64816 | 74.35184 |
| FAP-IL2v | 87.65834 | 12.34166 |
| PD1-IL2v Q126T | 13.58967 | 86.41033 |
| FAP-IL2v Q126T | 92.71401 | 7.285988 |
| FAP-IL2v + | 69.28097 | 30.71903 |
| parental anti-PD-1 | | |
| FAP-IL2v Q126T + | 84.53426 | 15.46574 |
| parental anti-PD-1 | | |

### Example 3E. IL-2R Signaling (STAT5-P) on Activated PD-1⁺ and PD-1⁻ CD4 T Cells Upon Treatment with Increasing Doses of PD-1-(LAG-3)-IL-2v Immunoconjugates

The potency and the cis/trans-signaling of a PD-1-IL-2v, PD1-LAG3-IL2v and FAP-IL2v immunoconjugates were measured as IL-2R signaling by treating activated PD-1 expressing (PD-1⁺) and PD-1 negative (PD-1⁻) (anti-PD-1 pre-treated) CD4 T cells with increasing concentrations of immunoconjugates. The purpose was to determine the dependency of the PD-1-(LAG3)-IL-2v immunoconjugates on the PD-1, and LAG-3, expression of the T cells in order to deliver IL-2R signaling.

For this CD4 T cells from healthy donor PBMCs were sorted with CD4 beads (#130-045-101, Miltenyi) and activated for 3 days in presence of 1 μg/ml plate-bound anti-CD3 (overnight pre-coated, clone OKT3, #317315, BioLegend) and 1 μg/ml of soluble anti-CD28 (clone CD28.2, #302923, BioLegend) antibodies to induce PD-1 expression. Three days later, the cells were harvested and washed several times to remove endogenous cytokines and half of the cells were labeled with Cell Trace Violet (CTV) (5 μM, 5 minutes at room temperature (RT); C34557, Thermo Scientific) and the other half were left unlabeled.

Then, the unlabeled cells were incubated with a saturating concentration of a competing anti-PD-1 and anti-LAG-3 antibodies (in-house molecule, 10 μg/ml) for 30 minutes at RT followed by several washing steps to remove the excess unbound anti-PD-1 antibody. Thereafter, the PD-1 pre-blocked unlabeled cells (25 μl, 6*10⁶ cells/ml) were co-cultured 1:1 with the PD-1 CTV-labeled cells (25 μl, 6×10⁶ cells/ml) in a V-bottom plate before being treated for 12 minutes at 37° C. with increasing concentrations of treatment immunoconjugates (50 μl, 1:10 dilution steps). To preserve the phosphorylation state, an equal amount of Phosphoflow Fix Buffer I (100 μl, 557870, BD Bioscience) was added after 12 minutes incubation with the various constructs to allow the IL-2R signaling upon binding to PD-1. The cells were then incubated for an additional 30 minutes at 37° C. for fixation before being permeabilized overnight at −80° C. with Phosphoflow PermBuffer III (558050, BD Bioscience). On the next day, STAT-5 in its phosphorylated form was stained for 30 minutes at 4° C. by using an anti-STAT-5P antibody (47/Stat5(pY694) clone, 562076, BD Bioscience).

The cells were acquired at the flow cytometer (FACS) BD-SymphonyA5 (BD Bioscience) instrument. The frequency of STAT-5P was determined with FlowJo (V10) and plotted with GraphPad Prism (v8).

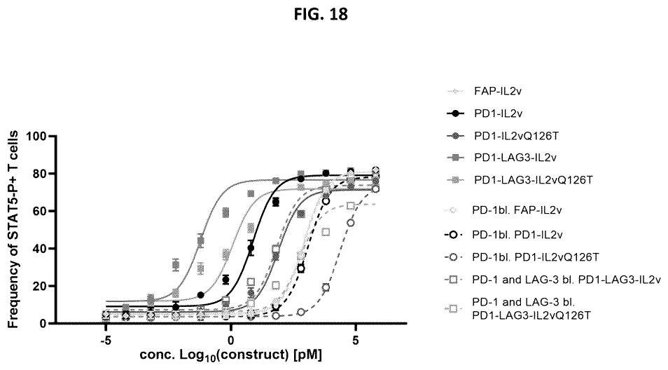

The data in FIG. 18 show the potency difference of PD1−, LAG3−, FAP-IL2v variants in signaling through the IL-2R on PD-1⁺, LAG-3⁺ and PD-1⁻, LAG-3⁻ CD4 T cells. The potency measurement in the PD1⁺, LAG-3⁺ CD4 T cells reflects the PD1-(LAG-3) mediated delivery of IL-2v versus the PD1-(LAG-3) independent delivery of IL-2v in PD1⁻, LAG-3⁻ CD4 T cells.

In Table 15, the STAT-5P EC50 fold increase between PD-1⁻ (LAG-3) mediated and PD-1, LAG-3 independent delivery of IL-2v of each PD1-IL2v immunoconjugate molecule was calculated by dividing the EC50 of the PD-1 (LAG-3) pre-blocked cells by the EC50 of PD1⁺, LAG-3⁺, T cells. This provides evidence on the strength of the PD-1-(LAG-3) dependent delivery of IL2v for each of the IL2v mutants. Furthermore, the EC50 fold increase between the various PD1-(LAG-3)-IL2v immunoconjugates and PD1-IL2v was calculated by dividing the EC50 of the new mutants by the EC50 of PD1-IL2v. This indicated the loss in potency of the PD1-IL2v immunoconjugates in signaling thorugh the IL-2R due to their reduced affinity. PD1-LAG-3-IL2v and PD1-LAG-3-IL2vQI26T have a higher potency, 126.6 folds and 6.5 folds respectively, on PD-1⁺, LAG-3⁺ T cells than PD1-IL2v due to the avidity gain of the PD-1 and LAG-3 co-targeting. However, also their potency on PD-1⁻, LAG3⁻ T cells is increased (FIG. 18 and Table 15). Nevertheless, the cis-activity window of PD1-LAG3-IL2v and PD1-LAG3-IL2vQ126T is of 1105 and 485 folds higher, respectively, on PD-1⁺, LAG-3⁺ T cells than on PD-1⁻, LAG3⁻ T cells versus the 158 and 305 folds of the PD1-IL2v and PD1-IL2vQ126T, respectively (FIG. 18 and Table 15).

TABLE 15
EC50 of the dose-response STAT-5 phosphorylation as frequencies for the IL-2v
mutants on PD-1⁺ LAG-3⁺ and PD-1⁻, LAG-3⁻ CD4 T cells. Mean ± SEM of 3 donors.

| | | | Cis-delivery: fold | |
|---|---|---|---|---|
| | | | increase in EC50 | |
| | EC50 (Mean ± SEM) | EC50 (Mean ± SEM) | EC50 [PD1/LAG-3 | Potency reduction vs |
| | on PD1+/LAG-3⁺ | on PD1/LAG-3⁻ | bl.]/EC50 | PD1-IL2v on PD-1+ |
| Compounds | T cells | blocked Tcells | [PD1⁺/LAG-3⁺] | LAG-3+ T cells |
| FAP-IL2v | 823 | 831.9 | 1.0 | 107.2732013 |
| PD1-IL2v | 7.672 | 1210 | 157.7 | 1 |
| PD1-IL2vQ126T | 77.03 | 23499 | 305.1 | 10.04040667 |
| PD1-LAG3-IL2v | 0.06035 | 66.72 | 1105.6 | 0.007866267 |
| PD1-LAG3- | 1.178 | 571.6 | 485.2 | 0.15354536 |
| IL2vQ126T | | | | |

### Example 3F. Rescue of T_{conv} Effector Function from T_{reg} Suppression Upon Treatment with PD-1-(LAG-3)-IL-2v Immunoconjugates

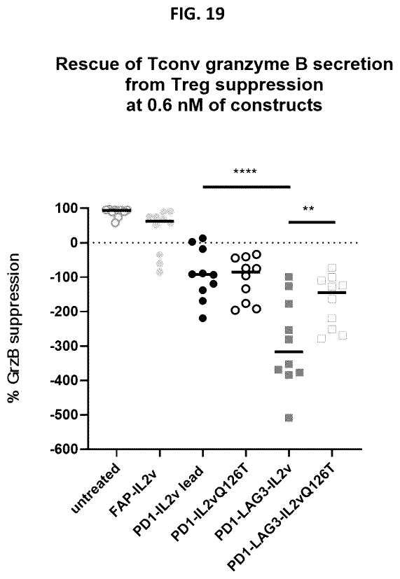

To assess the ability of PD1-LAG3-IL2v and PD1-LAG3-IL2vQ126T to rescue Tconv from Treg suppression, a suppressive-function assay was established where Tconv and Treg were cultured together for 5 days with CD4⁻ CD25⁻ from an unrelated donor to elicit an allospecific stimulation in presence or absence of immunoconjugates as previously described in example 3D. The data in FIG. 19 show that Treg suppress 88% of granzyme B secretion by Tconv when left untreated. PD1-IL2v and PD1-IL2vQ126T at 0.6 nM reduce the suppression to −92.2% and −106.3% respectively and therefore are able to not only rescue from Treg suppression but further boost the granzyme B secretion by Tconv to 192% and 206% respectively (FIG. 19 and Table 16). 0.6 nM of the untargeted version FAP-IL2v reduce the suppression to 33% and therefore is able to rescue 66.38% of the granzyme B secretion (FIG. 19 and Table 16). 0.6 nM of the PD1-LAG3-IL2v and PD1-LAG3-IL2vQ126T reduce the suppression to −293.25 and −171-4 respectively resulting in an increase in granzyme B secretion to 393% and 271.42% (FIG. 19 and Table 16). FIG. 19 shows the median and the individual values of 10 donors from independent experiments and Table 16 depicts the numerical median values. P is calculated using one-way ANOVA (*p<0.05, **p<0.01, ***p<0.001, ****p<0.0001).

TABLE 16
Percentage of Treg-mediated suppression of granzyme
B produced by Tconv and percentage of rescued granzyme
B production by T conv. Median of 10 donors.

| | Percentage of | Percentage of |
|---|---|---|
| Compounds | suppressed Granzyme | rescued Granzyme |
| at 0.6 nM | B production by T conv | B production by T conv |
| UT | 88.27167 | 11.72833 |
| FAP-IL2v | 33.61475 | 66.38525 |
| PD1-IL2v | −92.2398 | 192.2398 |
| PD1-IL2vQ126T | −106.32 | 206.3198 |
| PD1-LAG3-IL2v | −293.25 | 393.25 |
| PD1-LAG3- | −171.425 | 271.4246 |
| IL2vQ126T | | |

### Example 3G. Internalization of PD-1-(LAG-3)-IL-2v Immunoconjugates by Activated CD4 T Cells

IL-2 once bound to the IL-2R induces the internalization of the IL-2/IL2R complex, which could represent a sink for immunoconjugates affecting exposure. For this purpose PBMCs were isolated by density gradient centrifugation using Ficoll-Paque (Sigma-Aldrich). CD4 T cells were bead-sorted starting from 10⁸ PBMCs by using a CD4-positive selection kit (Miltenyi, #130-045-101) following manufacturer instructions. CD4 T cells were then seeded in a 12 well plate previously coated with 1 μg/ml anti-CD3 (overnight pre-coated, clone OKT3, #317315, BioLegend) at 2-4×10⁶ cells/well in RPMI 10% FBS in presence of 1 μg/ml of soluble anti-CD28 (clone CD28.2, #302923, BioLegend) and cultured for 3 days at 37° C.

Three days activated CD4 T cells were incubated in duplicates in FACS tubes in presence of either parental anti-PD-1, PD1-IL2v, PD1-LAG3-IL2v, FAP-IL2v, PD1-IL2vQ126T, PD1-LAG3-IL2vQ126T and FAP-IL2vQ126T for 30 minutes at 4° C. The cells were then washed, divided in two groups, one of which was incubated for 3 additional hours at 37° C. and the other one was immediately stained with a PE labelled anti-PGLALA secondary antibody and anti-CD4 antibody (eBioscience) before being fixed with BD Cell Fix. After the 3 hours incubations also the second group of the cells were stained with the PE labelled anti-PGLALA secondary antibody and anti-CD4 antibody before fixation. The cells were then acquired at LSRFortessa (BD Biosciences) and data analysis were performed with FlowJo (V10) and plotted with GraphPad Prism (v8). The expression levels of detectable antibody on the cell surface at 4° C. was compared to the one at 37° C. and frequency of positive cells at 37° C. were deducted from the frequency of positive cells at 4° C. to calculate the percentage of internalized molecules at 37° C. P is calculated using one-way ANOVA (*p<0.05, **p<0.01, ***p<0.001, ****p<0.0001).

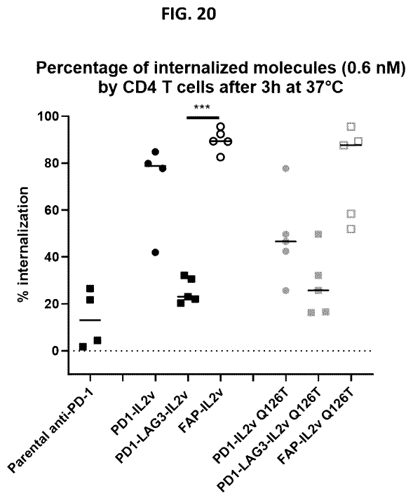

The parental anti-PD1 was used as negative control for internalization as it remains on the extracellular portion of the cellular membrane. 89.8% of FAP-IL2v and 76.5% of FAP-IL2vQ126T are internalized after 3 hours of incubation, followed by PD1-IL2v with 71%. Interestingly only 47.4% of PD1-IL2vQ126T is internalized after 3 hours due to the higher affinity for PD-1 and the further reduced affinity for the IL-2R. The internalization was further lowered to 25% by the targeting of the IL-2v to PD-1 and LAG-3 with the PD1-LAG3-IL2v, which remained unchanged (28%) by the further reduction in affinity to IL-2R with the IL-2vQ126T (FIG. 20 and Table 17).

TABLE 17
Percentage of internalized molecules at 0.6 nM by activated CD4
T cells after 3 hours of incubation at 37° C. Median of 4 donors

| Compounds at 0.6 nM | Percentage of internalization |
|---|---|
| Parental anti-PD-1 | 13.58815 |
| PD1-IL2v | 71.10301 |
| PD1-LAG3-IL2v | 25.65488 |
| FAP-IL2v | 89.8058 |
| PD1-IL2v Q126T | 48.44752 |
| PD1-LAG3-IL2v Q126T | 28.12816 |
| FAP-IL2v Q126T | 76.56525 |

### Example 4

### In Vivo Efficacy of Murine Surrogate PD1-IL2vQ126T Immuno-Conjugates, in a Syngeneic Model of Mouse Tumor Cell Line.—02-Fluc Subcutaneous Syngeneic Model

The murine surrogate PD1-IL2vQ126T immuno-conjugates was tested in the mouse pancreatic carcinoma cell line Panc02-Fluc, injected subcutaneously into Black 6-huIL2RBG transgenic mice.

Panc02-H7 cells (mouse pancreatic carcinoma) were originally obtained from the MD Anderson cancer center (Texas, USA) and after expansion deposited in the Roche-Glycart internal cell bank. Panc02-H7-Fluc cell line was produced in house by calcium transfection and sub-cloning techniques. Panc02-H7-Fluc was cultured in RPMI medium containing 10% FCS (Sigma), 500 ug/ml hygromicin and 1% of Glutamax. The cells were cultured at 37° C. in a water-saturated atmosphere at 5% CO2. Passage 14 was used for transplantation. Cell viability was 94.7%. 2×10⁵ cells per animal were injected subcutaneously in 100 μl of RPMI cell culture medium (Gibco) into the flank of mice using a 1 ml tuberculin syringe (BD Biosciences).

Female Black 6-huIL2RBG transgenics mice, aged 7-8 weeks at the start of the experiment (Breed at Charles Rivers, Lyon, France) were maintained under specific-pathogen-free condition with daily cycles of 12 h light/12 h darkness according to committed guidelines (GV-Solas; Felasa; TierschG). After arrival, animals were maintained for one week to get accustomed to the new environment and for observation. Continuous health monitoring was carried out on a regular basis.

Mice were injected subcutaneously on study day 0 with 2×10⁵ of Panc02-Fluc cells, randomized and weighed. Fifteen days after the tumor cell injection (tumor volume >100 mm³), mice were injected i.v. with muPD1-IL2vQ126T variant, muPD-IL2v or Vehicle once a week for three weeks. All mice were injected i.v. with 200 μl of the appropriate solution. The mice in the Vehicle group were injected with Histidine Buffer and the treatment groups with the muPD1-IL2vQ126T variant with 2 mg/kg iv qw or the muPD1-IL2v with 0.5 mg/kg iv qw for 3 weeks.

To obtain the proper amount of immunoconjugates per 200 μl, the stock solutions were diluted with Histidine Buffer when necessary.

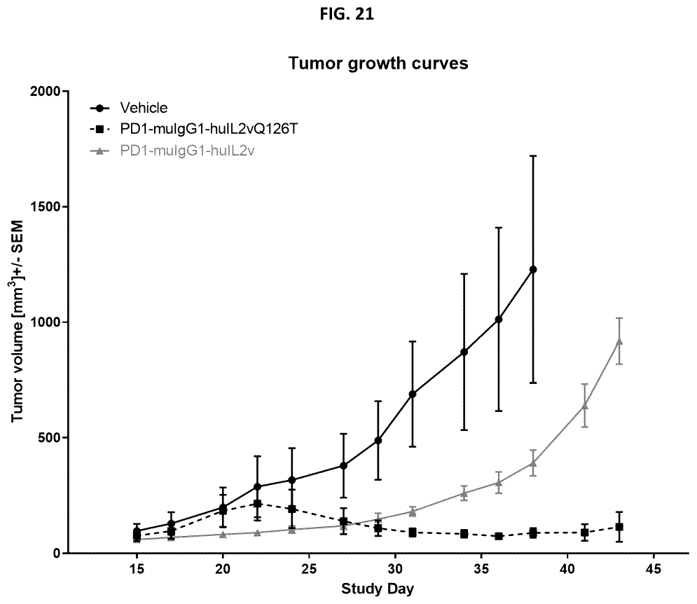

FIG. 21 shows that the muPD1-IL2vQ126T variant mediated superior efficacy in terms of tumor growth inhibition compared to the vehicle and muPD1-IL2v groups. The muPD1-IL2vQ126T variant injected mice tolerated well the treatment.

TABLE 18

| | | Dose/ | Concentration |
|---|---|---|---|
| Compound | Batch ID | mouse | (mg/mL) |
| PD1-IL2vQ126T | P1AG8304-001 | 40 μg | 2.1 (=stock |
| | | | solution) |
| PD1-IL2v | P1AG9991-003 | 10 μg | 1.1 (=stock |
| | | | solution) |

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. An immunoconjugate comprising a mutant IL-2 polypeptide and an antibody that binds to PD-1, wherein the mutant IL-2 polypeptide is a human IL-2 molecule comprising the amino acid substitutions F42A, Y45A, L72G and Q126T (numbering relative to the human IL-2 sequence SEQ ID NO: 90).

2. The immunoconjugate of claim 1, wherein the antibody comprises
(a) a heavy chain variable region (VH) comprising a CDR-H1 comprising the amino acid sequence of SEQ ID NO:74, a CDR-H2 comprising the amino acid sequence of SEQ ID NO:75, and a CDR-H3 comprising the amino acid sequence of SEQ ID NO:76, and
(b) a light chain variable region (VL) comprising a CDR-L1 comprising the amino acid sequence of SEQ ID NO:77, a CDR-L2 comprising the amino acid sequence of SEQ ID NO:78, and a CDR-L3 comprising the amino acid sequence of SEQ ID NO:79.

3. The immunoconjugate of claim 1,
wherein the antibody comprises (a) a heavy chain variable region (VH) comprising an amino acid sequence that is at least about 95%, 96%, 97%, 98%, 99% or 100% identical to the amino acid sequence of SEQ ID NO:80, and (b) a light chain variable region (VL) comprising an amino acid sequence that is at least about 95%, 96%, 97%, 98%, 99% or 100% identical to the amino acid sequence of SEQ ID NO:81.

4. The immunoconjugate of claim 1, wherein the mutant IL-2 polypeptide further comprises the amino acid substitution T3A and/or the amino acid substitution C125A.

5. The immunoconjugate of claim 1, wherein the mutant IL-2 polypeptide comprises the sequence of SEQ ID NO: 92.

6. The immunoconjugate of claim 1, wherein the immunoconjugate comprises not more than one mutant IL-2 polypeptide.

7. The immunoconjugate of claim 1, wherein the antibody comprises an Fc domain composed of a first and a second subunit.

8. The immunoconjugate of claim 7, wherein the Fc domain is an IgG₁ subclass Fc domain.

9. The immunoconjugate of claim 6, wherein the Fc domain is a human Fc domain.

10. (canceled)

11. The immunoconjugate of claim 7, wherein the Fc domain comprises a modification promoting the association of the first and the second subunit of the Fc domain.

12. The immunoconjugate of claim 7, wherein in the CH3 domain of the first subunit of the Fc domain an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

13. The immunoconjugate of claim 7, wherein in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A), each as numbered according to Kabat EU index.

14. The immunoconjugate of claim 13, wherein in the first subunit of the Fc domain additionally the serine residue at position 354 is replaced with a cysteine residue (S354C) or the glutamic acid residue at position 356 is replaced with a cysteine residue (E356C), and in the second subunit of the Fc domain additionally the tyrosine residue at position 349 is replaced by a cysteine residue (Y349C), each as numbered according to Kabat EU index.

15. The immunoconjugate of claim 7, wherein the mutant IL-2 polypeptide is fused at its amino-terminal amino acid to the carboxy-terminal amino acid of one of the subunits of the Fc domain, particularly the first subunit of the Fc domain, optionally through a linker peptide.

16. (canceled)

17. The immunoconjugate of claim 7, wherein the Fc domain comprises one or more amino acid substitutions that reduces binding to an Feγ receptor, and/or antibody-dependent cell-mediated cytotoxicity (ADCC).

18. The immunoconjugate of claim 17, wherein said one or more amino acid substitution is at one or more position selected from the group of L234, L235, and P329, as numbered according to Kabat EU index.

19. (canceled)

20. The immunoconjugate of claim 1, comprising a polypeptide comprising an amino acid sequence that is at least about 80%, 85%, 90%, 95%, 96%, 97%, 98%, 99% or 100% identical to the sequence of SEQ ID NO:21, a polypeptide comprising an amino acid sequence that is at least about 80%, 85%, 90%, 95%, 96%, 97%, 98%, 99% or 100% identical to the sequence of SEQ ID NO:22, and a polypeptide comprising an amino acid sequence that is at least about 80%, 85%, 90%, 95%, 96%, 97%, 98%, 99% or 100% identical to the sequence of SEQ ID NO:35.

21. (canceled)

22. One or more isolated polynucleotide encoding the immunoconjugate of claim 1.

23. One or more vector, particularly expression vector, comprising the polynucleotide(s) of claim 22.

24. A host cell comprising the polynucleotide(s) of claim 22.

25. A method of producing an immunoconjugate comprising a mutant IL-2 polypeptide and an antibody that binds to PD-1, comprising (a) culturing the host cell of claim 24 under conditions suitable for the expression of the immunoconjugate, and optionally (b) recovering the immunoconjugate.

26. (canceled)

27. A pharmaceutical composition comprising the immunoconjugate of claim 1 and a pharmaceutically acceptable carrier.

28.-32. (canceled)

33. A method of treating a disease in an individual, comprising administering to said individual a therapeutically effective amount of a composition comprising the immunoconjugate of claim 1 in a pharmaceutically acceptable form.

34. The method of claim 33, wherein said disease is cancer.

35. A method of stimulating the immune system of an individual, comprising administering to said individual an effective amount of a composition comprising the immunoconjugate of claim 1 in a pharmaceutically acceptable form.

36. (canceled)


SEQUENCE LISTING (100 sequences)

SEQ ID NO: 1 - protein, 215 aa, synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 447 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3 - protein, 594 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 4 - protein, 218 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 450 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 6 - protein, 597 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 7 - protein, 604 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 8 - protein, 604 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSAPASSSTKKTQLQLEHLLLTLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 9 - protein, 790 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGE

SEQ ID NO: 10 - protein, 841 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSSLVTTTDFQIQTEMAATMETSIFTTEGGGGSGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 11 - protein, 604 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSAPASSSTKKTQLQLEHLLLDLQMILNGINNYKNPKLTRMLTAKFAMPKKATELKHLQCLEEELKPLEEVLNGAHSKNFHFDPRDVVSNINVFVLELKGSETTFMCEYADETATIVEFLNRWITFAQSIISTLT

SEQ ID NO: 12 - protein, 218 aa, synthetic construct
DIQMTQSPSSLSASVGDRVTITCRASESVDTSDNSFIHWYQQKPGKSPKLLIYRSSTLESGVPSRFSGSGSGTDFTLTISSLQPEDFATYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 450 aa, synthetic construct; identical to SEQ ID NO: 5

SEQ ID NO: 14 - protein, 597 aa, synthetic construct; identical to SEQ ID NO: 6

SEQ ID NO: 15 - protein, 214 aa, synthetic construct
DIQMTQSPSSLSASVGDRVTITCRASQGISNDLAWYQQKPGKAPKLLIYAASSLQSGVPSRFSGSGSGTDFTLTISSLQPEDFATYYCQQTYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 451 aa, synthetic construct
EVQLLESGGGLVQPGGSLRLSCAASGFTFSSYAMSWVRQAPGKGLEWVSAIGIGGGGTYYADSVKGRFTISRDNSKNTLYLQMNSLRAEDTAVYHCAKGASFDFINFFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 17 - protein, 598 aa, synthetic construct
EVQLLESGGGLVQPGGSLRLSCAASGFTFSSYAMSWVRQAPGKGLEWVSAIGIGGGGTYYADSVKGRFTISRDNSKNTLYLQMNSLRAEDTAVYHCAKGASFDFINFFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 18 - protein, 218 aa, synthetic construct; identical to SEQ ID NO: 4

SEQ ID NO: 19 - protein, 450 aa, synthetic construct; identical to SEQ ID NO: 5

SEQ ID NO: 20 - protein, 597 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISDINVIVLELKGSETTFMCEYADETATIVEFLNRWITFAQSIISTLT

SEQ ID NO: 21 - protein, 218 aa, synthetic construct; identical to SEQ ID NO: 4

SEQ ID NO: 22 - protein, 450 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 23 - protein, 597 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTSKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 24 - protein, 597 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQVQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 25 - protein, 597 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQAQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 26 - protein, 597 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLVDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 27 - protein, 597 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKAPELKHLQCLEEELKPLEEVLNGAQSKNFHLRPRDLISNINVIVLELKGSETTFMCEYADETATIVEFLNRWITFAQSIISTLT

SEQ ID NO: 28 - protein, 597 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFSLRPRDLISNINVIVLELKGSETTFMCEYADETATIVEFLNRWITFAQSIISTLT

SEQ ID NO: 29 - protein, 597 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHARPRDLISNINVIVLELKGSETTFMCEYADETATIVEFLNRWITFAQSIISTLT

SEQ ID NO: 30 - protein, 597 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISAINVIVLELKGSETTFMCEYADETATIVEFLNRWITFAQSIISTLT

SEQ ID NO: 31 - protein, 597 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISQINVIVLELKGSETTFMCEYADETATIVEFLNRWITFAQSIISTLT

SEQ ID NO: 32 - protein, 597 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYAAETATIVEFLNRWITFAQSIISTLT

SEQ ID NO: 33 - protein, 597 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFANSIISTLT

SEQ ID NO: 34 - protein, 597 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAESIISTLT

SEQ ID NO: 35 - protein, 597 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFATSIISTLT

SEQ ID NO: 36 - protein, 604 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSAPASSSTKKTQLQLVHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 37 - protein, 604 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLALKGSETTFMCEYADETATIVEFLNRWITFAQSIISTLT

SEQ ID NO: 38 - protein, 604 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSAPASSSTKKTQAQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 39 - protein, 604 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSAPASSSTKKTQAQLEHLLADLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 40 - protein, 604 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSAPASSSTKKTQAQLEHLLLDLQMILNGINNYKNPKLTRMLTAKFAMPKKATELKHLQCLEEELKPLEEVLNGAQSKNFHARPRDLISNINVIVLELKGSETTFMCEYADETATIVEFLNRWITFAQSIISTLT

SEQ ID NO: 41 - protein, 604 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSAPASSSTKKTQLQLEHLLVDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 42 - protein, 604 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISTINVIVLELKGSETTFMCEYADETATIVEFLNRWITFAQSIISTLT

SEQ ID NO: 43 - protein, 604 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLKRWITFAQSIISTLT

SEQ ID NO: 44 - protein, 604 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSAPASSSTKKTQLQLEHLLLDLAMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 45 - protein, 604 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFATSIISTLT

SEQ ID NO: 46 - protein, 604 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLIANINVIVLELKGSETTFMCEYADETATIVEFLNRWITFAQSIISTLT

SEQ ID NO: 47 - protein, 604 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIIATLT

SEQ ID NO: 48 - protein, 604 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLK

SEQ ID NO: 49 - protein, 215 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRDSFSIHQNVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 50 - protein, 448 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKELYREYMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 51 - protein, 595 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKELYREYMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTSKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 52 - protein, 595 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKELYREYMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQAQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 53 - protein, 595 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKELYREYMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLVDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 54 - protein, 595 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKELYREYMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHARPRDLISNINVIVLELKGSETTFMCEYADETATIVEFLNRWITFAQSIISTLT

SEQ ID NO: 55 - protein, 214 aa, synthetic construct
DIQMTQSPSSLSASVGDRVTITCRASQDISGALNWYQQKPGKAPNLLIYAASSLQSGVPSRFSGSGSGTDYTLTISSLQPEDFATYYCQQANSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 451 aa, synthetic construct
QEQLVQSGAEVKKPGESLKISCKGSGFDFNTFWMTWVRQMPGKGLEWMGSIHGGSGSRDYSPSFQGQVTISADIDQSTAYLQWSSLKASDTAMYYCARPGYRSWSKTFDLWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 57 - protein, 598 aa, synthetic construct
QEQLVQSGAEVKKPGESLKISCKGSGFDFNTFWMTWVRQMPGKGLEWMGSIHGGSGSRDYSPSFQGQVTISADIDQSTAYLQWSSLKASDTAMYYCARPGYRSWSKTFDLWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFATSIISTLT

SEQ ID NO: 58 - protein, 218 aa, synthetic construct
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 59 - protein, 443 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPG

SEQ ID NO: 60 - protein, 591 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 61 - protein, 218 aa, synthetic construct; identical to SEQ ID NO: 58

SEQ ID NO: 62 - protein, 443 aa, synthetic construct; identical to SEQ ID NO: 59

SEQ ID NO: 63 - protein, 591 aa, synthetic construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGGAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFATSIISTLT

SEQ ID NO: 64 - protein, 227 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 214 aa, synthetic construct
DIQMTQSPSSLSASVGDRVTITCRASQSISSYLNWYQQKPGKAPKLLIYAASTLQSGVPSRFSGSGSGTDFTLTISSLQPEDFATYYCQQTYSSPLTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 451 aa, synthetic construct
EVQLLESGGGLVQPGGSLRLSCAASGFIFDDYTMNWVRQAPGKGLEWVAVISWDGGGTYYTDSVKGRFTISRDDFKNTLYLQMNSLRAEDTAVYYCAKGLTDTTLYGSDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 67 - protein, 590 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 68 - protein, 227 aa, synthetic construct; identical to SEQ ID NO: 64

SEQ ID NO: 69 - protein, 214 aa, synthetic construct; identical to SEQ ID NO: 65

SEQ ID NO: 70 - protein, 451 aa, synthetic construct; identical to SEQ ID NO: 66

SEQ ID NO: 71 - protein, 590 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFATSIISTLT

SEQ ID NO: 72 - protein, 218 aa, synthetic construct; identical to SEQ ID NO: 4

SEQ ID NO: 73 - protein, 450 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 5 aa, synthetic construct
SYTMS

SEQ ID NO: 75 - protein, 17 aa, synthetic construct
TISGGGRDIYYPDSVKG

SEQ ID NO: 76 - protein, 11 aa, synthetic construct
LTGRVYFALDS

SEQ ID NO: 77 - protein, 15 aa, synthetic construct
KASESVDTSDNSFIH

SEQ ID NO: 78 - protein, 7 aa, synthetic construct
RSSTLES

SEQ ID NO: 79 - protein, 9 aa, synthetic construct
QQNYDVPWT

SEQ ID NO: 80 - protein, 120 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSS

SEQ ID NO: 81 - protein, 111 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIK

SEQ ID NO: 82 - protein, 5 aa, synthetic construct
DYTMN

SEQ ID NO: 83 - protein, 17 aa, synthetic construct
VISWDGGGTYYTDSVKG

SEQ ID NO: 84 - protein, 12 aa, synthetic construct
GLTDTTLYGSDY

SEQ ID NO: 85 - protein, 11 aa, synthetic construct
RASQSISSYLN

SEQ ID NO: 86 - protein, 7 aa, synthetic construct
AASTLQS

SEQ ID NO: 87 - protein, 9 aa, synthetic construct
QQTYSSPLT

SEQ ID NO: 88 - protein, 121 aa, synthetic construct
EVQLLESGGGLVQPGGSLRLSCAASGFIFDDYTMNWVRQAPGKGLEWVAVISWDGGGTYYTDSVKGRFTISRDDFKNTLYLQMNSLRAEDTAVYYCAKGLTDTTLYGSDYWGQGTLVTVSS

SEQ ID NO: 89 - protein, 107 aa, synthetic construct
DIQMTQSPSSLSASVGDRVTITCRASQSISSYLNWYQQKPGKAPKLLIYAASTLQSGVPSRFSGSGSGTDFTLTISSLQPEDFATYYCQQTYSSPLTFGGGTKVEIK

SEQ ID NO: 90 - protein, 133 aa, Homo sapiens [sequence omitted]

SEQ ID NO: 91 - protein, 133 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 92 - protein, 133 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFATSIISTLT

SEQ ID NO: 93 - protein, 15 aa, synthetic construct
GGGGSGGGGSGGGGS

SEQ ID NO: 94 - protein, 20 aa, Homo sapiens
MYRMQLLSCIALSLALVTNS

SEQ ID NO: 95 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 96 - protein, 268 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 97 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 98 - protein, 133 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISDINVIVLELKGSETTFMCEYADETATIVEFLNRWITFAQSIISTLT

SEQ ID NO: 99 - protein, 133 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISQINVIVLELKGSETTFMCEYADETATIVEFLNRWITFAQSIISTLT

SEQ ID NO: 100 - protein, 597 aa, synthetic construct; identical to SEQ ID NO: 20